SEQUENCE LISTING (83 sequences)

SEQ ID NO: 1 - protein, 785 aa, Homo sapiens
MPKGRQKVPHLDAPLGLPTCLWLELAGLFLLVPWVMGLAGTGGPDGQGTGGPSWAVHLESLEGDGEEETLEQQADALAQAAGLVNAGRIGELQGHYLFVQPAGHRPALEVEAIRQQVEAVLAGHEAVRWHSEQRLLRRAKRSVHFNDPKYPQQWHLNNRRSPGRDINVTGVWERNVTGRGVTVVVVDDGVEHTIQDIAPNYSPEGSYDLNSNDPDPMPHPDVENGNHHGTRCAGEIAAVPNNSFCAVGVAYGSRIAGIRVLDGPLTDSMEAVAFNKHYQINDIYSCSWGPDDDGKTVDGPHQLGKAALQHGVIAGRQGFGSIFVVASGNGGQHNDNCNYDGYANSIYTVTIGAVDEEGRMPFYAEECASMLAVTFSGGDKMLRSIVTTDWDLQKGTGCTEGHTGTSAAAPLAAGMIALMLQVRPCLTWRDVQHIIVFTATRYEDRRAEWVTNEAGFSHSHQHGFGLLNAWRLVNAAKIWTSVPYLASYVSPVLKENKAIPQSPRSLEVLWNVSRMDLEMSGLKTLEHVAVTVSITHPRRGSLELKLFCPSGMMSLIGAPRSMDSDPNGFNDWTFSTVRCWGERARGTYRLVIRDVGDESFQVGILRQWQLTLYGSVWSAVDIRDRQRLLESAMSGKYLHDDFALPCPPGLKIPEEDGYTITPNTLKTLVLVGCFTVFWTVYYMLEVYLSQRNVASNQVCRSGPCHWPHRSRKAKEEGTELESVPLCSSKDPDEVETESRGPPTTSDLLAPDLLEQGDWSLSQNKSALDCPHQHLDVPHGKEEQIC

SEQ ID NO: 2 - RNA, 3940 nt, Homo sapiens
  source 1..3940: mol_type = mRNA
gatgctgcgggagacccctgagatacaagtggactctcccggggcgcggggaagaagggagagccgactgcgagaaggaaggcgacctgggtgggcaggatcccctctgggaagattacagggacagtcggggccatggcggtcagaaggagaagagcaacgagtggcttttaaaggtcggaaccgaaatgccctttacagaggacgccatgaaggagagccttctgaagagggaccatgtggaagatgccccgaggactcggggaggcggagcccgcgggttcagcccccagcgcccgcacggcccctgcctgcgtaggccgcccgcgggagccggaccccgggcgccggttgctgtggcccgcgggggtcgccgggccgcaggcgccgcgctgattggctgcgccgcgggcggaagtgatgctgctgtcactgctcctccggctcccgggaagccgcgagtttccgcagggaggcggcggcagctgcggcggggccggtctgtagcctttgctgagtcaacactactcacagtgaaagtgatgccttttctccatttgcacaacatgagtgtgacgtggttagccagaccaggagctcaacctcatgtagaatccagtccactgctctgatgccgaaggggaggcagaaagtgccacacttggatgcccccctgggcctgcccacctgcctctggctggaattagccgggctcttcttactggttccctgggtcatgggcctggcagggacaggtgggcctgatggccagggcacaggggggccgagctgggctgtgcacctggaaagcctggaaggtgacggggaggaagagactctggagcagcaggcggatgccttggcccaggcagcagggctggtgaatgctggacgcatcggagagcttcaggggcactacctctttgtccagcctgctgggcacaggccggccctggaggtggaggccatccggcagcaggtggaggctgtgttggctgggcatgaagctgtgcgctggcactcagagcagaggctgctaaggcgggccaagcgcagcgtccacttcaacgaccccaagtacccgcagcaatggcacctgaataaccgacggagcccgggcagggacatcaacgtgacgggtgtgtgggaacgcaatgtgactgggcgaggggtgacggtggtggtagtggatgacggagtggaacacaccatccaggacattgcacccaactatagccctgagggtagctatgacctcaactctaatgaccctgaccccatgccccacccggatgtggagaatggcaaccaccatggcacgcgatgtgcaggagagatcgcggctgtgcccaacaacagcttctgtgccgtgggcgtggcctacgggagccgcatcgcaggtatccgggtactggatggacctctcacagacagcatggaggcagtggcgttcaacaagcactatcagatcaatgacatctacagctgcagctggggaccagatgacgatgggaagacagtggatggcccccatcagcttggaaaggctgccttacaacatggggtgattgctggtcgccagggctttgggagcatctttgtggtagccagtggcaacggaggccaacacaacgacaactgcaactacgatggctacgccaactccatctacaccgtcaccataggagctgtggatgaggagggacgcatgcctttctatgcagaagaatgtgcctccatgctggcagtcaccttcagtggtggggacaagatgcttcggagcattgtgaccactgactgggaccttcagaagggcactggctgcactgagggccacacagggacctcagctgcagcgcctctggcagctggcatgatagccttaatgctgcaggtgcggccctgcctcacgtggcgtgacgtccagcacatcattgtcttcacagccacccggtatgaggatcgccgtgcagagtgggtcaccaacgaggcaggcttcagccatagccaccagcacggtttcggcctcctcaacgcctggaggctcgtgaatgcagccaagatctggacatctgtcccttacttagcatcctacgtcagtcccgtgttaaaagaaaacaaggcgattccgcagtccccccgttccctggaggtcctgtggaatgtcagcaggatggacctggagatgtcagggctgaagaccctggagcatgtggcagtgacagtctccatcactcacccacggcgcggcagcttggagctgaagctgttctgccccagtggcatgatgtccctcatcggcgccccccgcagcatggactcggatcccaacggcttcaatgactggaccttctccactgtgcgatgctggggggagagagcccgagggacctacaggcttgtcatcagggatgtcggggatgagtcattccaggtcggcatcctccggcaatggcagctgaccctatatggctctgtgtggagtgcagtagacatcagggacagacaaaggctgttagagagtgccatgagtggaaaatacctgcacgatgacttcgccctgccctgcccaccggggctgaaaattcctgaggaagatggttacaccatcacccccaacaccctcaagaccctggtgctggtaggctgtttcaccgtcttctggactgtttactacatgctggaagtatatttgagccagaggaatgtggcttccaatcaagtttgtaggagtggaccctgccactggccccatcggagccggaaagccaaggaggaagggacagagctagaatcagtgccactttgcagcagcaaggatccagacgaagtggaaacagagagcaggggccctcccaccacctctgacctccttgccccagacctgctggagcaaggggactggagcctgtcccagaacaagagcgccctggactgccctcatcagcacctagacgtaccgcacgggaaggaggagcagatctgctgacctcagggcctgacagtgtgggacaggctcttctttcccaaaattagggagctcttgacagaaagcagttctgatgcttacatctggaatctgaggcatcctctgactccactcaaagagggtgagggccttcttaagatacaaatggtggaggattgctgccagagaagtctggtcagagccacagggtctgcctccagccaaacgggagcttttggtgagaaggtgttggacaggggattggcgcccccctttggtttggcctccatcctcatctctcttgggccaagccagctgcctaggtcccccaagcatgggggaccccttcccacatataagttgagaaggtgcctgccatagccaggagcgcatctcaatggaaacatcactggggtcacttgggaagaggacttcggggtagaggctgggaggagcccctggacatgcctgtcctgaaagcggctgcctccattatccattcccaagatgcctgatcagaaaccaaccatgaatgaacccctggctccttcaccacccccacgattggtatgatgctgccggcacagctgggatacacacggctcccccaggcctgagctgcttcactagggaatcctgcggcaggactgcagagcagatggcagatgcacatgttggaggagagagccttgggagccactgccactccagtcctgccaccaccctgtcttcctctgcaagtgctcagggaaatggccttcccgccggaggccagctatctgcctgacaggctgtgactcttctctcaaccttggccttctcccctcttctgagctagttggttgaattttttttaatgcttaagatttgtttttctcttttcacagcaacattttcttgaatttttttctgcacagcttttccaaaataaaaaccttccaaacaataaaaaaaaaaaaaaaaaaaaaaaaa

SEQ ID NO: 3 - RNA, 4649 nt, Homo sapiens
  source 1..4649: mol_type = mRNA
gatgctgcgggagacccctgagatacaagtggactctcccggggcgcggggaagaagggagagccgactgcgagaaggaaggcgacctgggtgggcaggatcccctctgggaagattacagggacagtcggggccatggcggtcagaaggagaagagcaacgagtggcttttaaaggtcggaaccgaaatgccctttacagaggacgccatgaaggagagccttctgaagagggaccatgtggaagatgccccgaggactcggggaggcggagcccgcgggttcagcccccagcgcccgcacggcccctgcctgcgtaggccgcccgcgggagccggaccccgggcgccggttgctgtggcccgcgggggtcgccgggccgcaggcgccgcgctgattggctgcgccgcgggcggaagtgatgctgctgtcactgctcctccggctcccgggaagccgcgagtttccgcagggaggcggcggcagctgcggcggggccggtctgtagcctttgctgagtcaacactactcacagtgaaagtgatgccttttctccatttgcacaacatgagtgtgacgtggttagccagaccaggagctcaacctcatgtagaatccagtccactgctctgatgccgaaggggaggcagaaagtgccacacttggatgcccccctgggcctgcccacctgcctctggctggaattagccgggctcttcttactggttccctgggtcatgggcctggcagggacaggtgggcctgatggccagggcacaggggggccgagctgggctgtgcacctggaaagcctggaaggtgacggggaggaagagactctggagcagcaggcggatgccttggcccaggcagcagggctggtgaatgctggacgcatcggagagcttcaggggcactacctctttgtccagcctgctgggcacaggccggccctggaggtggaggccatccggcagcaggtggaggctgtgttggctgggcatgaagctgtgcgctggcactcagagcagaggctgctaaggcgggccaagcgcagcgtccacttcaacgaccccaagtacccgcagcaatggcacctgaataaccgacggagcccgggcagggacatcaacgtgacgggtgtgtgggaacgcaatgtgactgggcgaggggtgacggtggtggtagtggatgacggagtggaacacaccatccaggacattgcacccaactatagccctgagggtagctatgacctcaactctaatgaccctgaccccatgccccacccggatgtggagaatggcaaccaccatggcacgcgatgtgcaggagagatcgcggctgtgcccaacaacagcttctgtgccgtgggcgtggcctacgggagccgcatcgcaggtatccgggtactggatggacctctcacagacagcatggaggcagtggcgttcaacaagcactatcagatcaatgacatctacagctgcagctggggaccagatgacgatgggaagacagtggatggcccccatcagcttggaaaggctgccttacaacatggggtgattgctggtcgccagggctttgggagcatctttgtggtagccagtggcaacggaggccaacacaacgacaactgcaactacgatggctacgccaactccatctacaccgtcaccataggagctgtggatgaggagggacgcatgcctttctatgcagaagaatgtgcctccatgctggcagtcaccttcagtggtggggacaagatgcttcggagcattgtgaccactgactgggaccttcagaagggcactggctgcactgagggccacacagggacctcagctgcagcgcctctggcagctggcatgatagccttaatgctgcaggtgcggccctgcctcacgtggcgtgacgtccagcacatcattgtcttcacagccacccggtatgaggatcgccgtgcagagtgggtcaccaacgaggcaggcttcagccatagccaccagcacggtttcggcctcctcaacgcctggaggctcgtgaatgcagccaagatctggacatctgtcccttacttagcatcctacgtcagtcccgtgttaaaagaaaacaaggcgattccgcagtccccccgttccctggaggtcctgtggaatgtcagcaggatggacctggagatgtcagggctgaagaccctggagcatgtggcagtgacagtctccatcactcacccacggcgcggcagcttggagctgaagctgttctgccccagtggcatgatgtccctcatcggcgccccccgcagcatggactcggatcccaacggcttcaatgactggaccttctccactgtgcgatgctggggggagagagcccgagggacctacaggcttgtcatcagggatgtcggggatgagtcattccaggtcggcatcctccggcaatggcagctgaccctatatggctctgtgtggagtgcagtagacatcagggacagacaaaggctgttagagagtgccatgagtggaaaatacctgcacgatgacttcgccctgccctgcccaccggggctgaaaattcctgaggaagatggttacaccatcacccccaacaccctcaagaccctggtgctggtaggctgtttcaccgtcttctggactgtttactacatgctggaagtatatttgagccagaggaatgtggcttccaatcaagtttgtaggagtggaccctgccactggccccatcggagccggaaagccaaggaggaagggacagagctagaatcagtgccactttgcagcagcaaggatccagacgaagtggaaacagagagcaggggccctcccaccacctctgacctccttgccccagacctgctggagcaaggggactggagcctgtcccagaacaagagcgccctggactgccctcatcagcacctagacgtaccgcacgggaaggaggagcagatctgctgacctcagggcctgacagtgtgggacaggctcttctttcccaaaattagggagctcttgacagaaagcagttctgatgcttacatctggaatctgaggcatcctctgactccactcaaagagggtgagggccttcttaagatacaaatggtggaggattgctgccagagaagtctggtcagagccacagggtctgcctccagccaaacgggagcttttggtgagaaggtgttggacaggggattggcgcccccctttggtttggcctccatcctcatctctcttgggccaagccagctgcctaggtcccccaagcatgggggaccccttcccacatataagttgagaaggtgcctgccatagccaggagcgcatctcaatggaaacatcactggggtcacttgggaagaggacttcggggtagaggctgggaggagcccctggacatgcctgtcctgaaagcggctgcctccattatccattcccaagatgcctgatcagaaaccaaccatgaatgaacccctggctccttcaccacccccacgattggtatgatgctgccggcacagctgggatacacacggctcccccaggcctgagctgcttcactagggaatcctgcggcaggactgcagagcagatggcagatgcacatgttggaggagagagccttgggagccactgccactccagtcctgccaccaccctgtcttcctctgcaagtgctcagggaaatggccttcccgccggaggccagctatctgcctgacaggctgtgactcttctctcaaccttggccttctcccctcttctgagctagttggttgaattttttttaatgcttaagatttgtttttctcttttcacagcaacattttcttgaatttttttctgcacagcttttccaaaataaaaaccttccaaacaattctgccactcgcaatctctcctttcctctgtctccccaaagccactgcatgctgctgagtggctcctttttcccttcggtccccaagattcactctcctgttctatggcctgtttcctctgaaagaatctgactgtcctggaatcttggcacatgaagggtccttcagaggacggcagccaggaacacatacaagcccgagatgcctgcctgtcccctgcgcagatctgaaaccataacaaaagtgaccagggcaaatcaaactctgccagccaggcctgagaaaagattgattttttttttttttttttgagttccaaccccaaaaatattccaggacaagatagggggcaggctgggctggttcttcttcaatgggcttttgccgccaaggaggacagtggacttggcccacatccaggctacccccagcccgcttctcccctgcttagcccagggaggagacagtagaggtgatgggggcagccgggagggaggggccaaggcagtgatgctttcgggtaagaagttgggctgaggctgggggcacagggaggagctgcccaggtgcagttaccattgctcagtgacagagcctcaaagcttggccagggctgaaggtaccacacgggtgtgggtgaggcaggctaagcgggatggctgcagccaaggagctgggggagggccgggctcagggctagccctctccgctctaactgatgatctgccgaggtc

SEQ ID NO: 4 - RNA, 3332 nt, Homo sapiens
  source 1..3332: mol_type = mRNA
cactgctcctccggctcccgggaagccgcgagtttccgcagggaggcggcggcagctgcggcggggccggtctgtagcctttgctgagtcaacactactcacagtgaaagtgatgccttttctccatttgcacaacatgagtgtgacgtggttagccagaccaggagctcaacctcatgtagaatccagtccactgctctgatgccgaaggggaggcagaaagtgccacacttggatgcccccctgggcctgcccacctgcctctggctggaattagccgggctcttcttactggttccctgggtcatgggcctggcagggacaggtgggcctgatggccagggcacaggggggccgagctgggctgtgcacctggaaagcctggaaggtgacggggaggaagagactctggagcagcaggcggatgccttggcccaggcagcagggctggtgaatgctggacgcatcggagagcttcaggggcactacctctttgtccagcctgctgggcacaggccggccctggaggtggaggccatccggcagcaggtggaggctgtgttggctgggcatgaagctgtgcgctggcactcagagcagaggctgctaaggcgggccaagcgcagcgtccacttcaacgaccccaagtacccgcagcaatggcacctgaataaccgacggagcccgggcagggacatcaacgtgacgggtgtgtgggaacgcaatgtgactgggcgaggggtgacggtggtggtagtggatgacggagtggaacacaccatccaggacattgcacccaactatagccctgagggtagctatgacctcaactctaatgaccctgaccccatgccccacccggatgtggagaatggcaaccaccatggcacgcgatgtgcaggagagatcgcggctgtgcccaacaacagcttctgtgccgtgggcgtggcctacgggagccgcatcgcaggtatccgggtactggatggacctctcacagacagcatggaggcagtggcgttcaacaagcactatcagatcaatgacatctacagctgcagctggggaccagatgacgatgggaagacagtggatggcccccatcagcttggaaaggctgccttacaacatggggtgattgctggtcgccagggctttgggagcatctttgtggtagccagtggcaacggaggccaacacaacgacaactgcaactacgatggctacgccaactccatctacaccgtcaccataggagctgtggatgaggagggacgcatgcctttctatgcagaagaatgtgcctccatgctggcagtcaccttcagtggtggggacaagatgcttcggagcattgtgaccactgactgggaccttcagaagggcactggctgcactgagggccacacagggacctcagctgcagcgcctctggcagctggcatgatagccttaatgctgcaggtgcggccctgcctcacgtggcgtgacgtccagcacatcattgtcttcacagccacccggtatgaggatcgccgtgcagagtgggtcaccaacgaggcaggcttcagccatagccaccagcacggtttcggcctcctcaacgcctggaggctcgtgaatgcagccaagatctggacatctgtcccttacttagcatcctacgtcagtcccgtgttaaaagaaaacaaggcgattccgcagtccccccgttccctggaggtcctgtggaatgtcagcaggatggacctggagatgtcagggctgaagaccctggagcatgtggcagtgacagtctccatcactcacccacggcgcggcagcttggagctgaagctgttctgccccagtggcatgatgtccctcatcggcgccccccgcagcatggactcatggctctgtgtggagtgcagtagacatcagggacagacaaaggctgttagagagtgccatgagtggaaaatacctgcacgatgacttcgccctgccctgcccaccggggctgaaaattcctgaggaagatggttacaccatcacccccaacaccctcaagaccctggtgctggtaggctgtttcaccgtcttctggactgtttactacatgctggaagtatatttgagccagaggaatgtggcttccaatcaagtttgtaggagtggaccctgccactggccccatcggagccggaaagccaaggaggaagggacagagctagaatcagtgccactttgcagcagcaaggatccagacgaagtggaaacagagagcaggggccctcccaccacctctgacctccttgccccagacctgctggagcaaggggactggagcctgtcccagaacaagagcgccctggactgccctcatcagcacctagacgtaccgcacgggaaggaggagcagatctgctgacctcagggcctgacagtgtgggacaggctcttctttcccaaaattagggagctcttgacagaaagcagttctgatgcttacatctggaatctgaggcatcctctgactccactcaaagagggtgagggccttcttaagatacaaatggtggaggattgctgccagagaagtctggtcagagccacagggtctgcctccagccaaacgggagcttttggtgagaaggtgttggacaggggattggcgcccccctttggtttggcctccatcctcatctctcttgggccaagccagctgcctaggtcccccaagcatgggggaccccttcccacatataagttgagaaggtgcctgccatagccaggagcgcatctcaatggaaacatcactggggtcacttgggaagaggacttcggggtagaggctgggaggagcccctggacatgcctgtcctgaaagcggctgcctccattatccattcccaagatgcctgatcagaaaccaaccatgaatgaacccctggctccttcaccacccccacgattggtatgatgctgccggcacagctgggatacacacggctcccccaggcctgagctgcttcactagggaatcctgcggcaggactgcagagcagatggcagatgcacatgttggaggagagagccttgggagccactgccactccagtcctgccaccaccctgtcttcctctgcaagtgctcagggaaatggccttcccgccggaggccagctatctgcctgacaggctgtgactcttctctcaaccttggccttctcccctcttctgagctagttggttgaattttttttaatgcttaagatttgtttttctcttttcacagcaacattttcttgaatttttttctgcacagcttttccaaaataaaaaccttcc

SEQ ID NO: 5 - protein, 783 aa, Rattus norvegicus
MPKGRQKVPRLDARLGLPICLCLELAIFFLVPQVMGLTEAGGLDTLGAGGLSWAVHLDSLEGERKEESLIQQANAVAQAAGLVNAGRIGELQGHYLFVQPAGHGQAMEAEAMRQQAEAVLAKHEAVRWHSEQRLLKRAKRSIHFNDPKYPQQWHLNNRRSPGRDINVTGVWERNVTGRGVTVVVVDDGVEHTVQDIAPNYSPEGSYDLNSNDPDPMPHPDEENGNHHGTRCAGEIAAVPNNSFCAVGVAYGSRIAGIRVLDGPLTDSMEAVAFNKHYQINDIYSCSWGPDDDGKTVDGPHQLGKAALQHGVMAGRQGFGSIFVVASGNGGQHNDNCNYDGYANSIYTVTIGAVDEEGRMPFYAEECASMLAVTFSGGDKMLRSIVTTDWDLQKGTGCTEGHTGTSAAAPLAAGMIALMLQVRPCLTWRDVQHIIVFTATQYEDHRADWLTNEAGFSHSHQHGFGLLNAWRLVNAAKIWTSVPYLASYVSPMLKENKAVPRSPHSLEVLWNVSRTDLEMSGLKTLEHVAVTVSITHPRRGSLELKLFCPSGMMSLIGAPRSMDSDPNGFNDWTFSTVRCWGERARGVYRLVIRDVGDEPLQVGILQQWQLTLYGSTWSPVDIKDRQSLLESAMSGKYLHDDFTLPCPPGLKIPEEDGYSITPNTLKTLVLVGCFSVFWTIYYMLEVCLSQRSKASTHGCRRGCCPWPPQSQNSKEVGTALESMPLCSSKDLDGVDSEHGDCTTASSLLAPELLGEADWSLSQNSKSDLDCPPHQPPDLKDGQIC

SEQ ID NO: 6 - RNA, 3485 nt, Rattus norvegicus
  source 1..3485: mol_type = mRNA
gaagtgaagttgctgtcagtggcgcctccggctcccggggaagctgagagtttccgcaggtggcggctgcggcggcggcggcagcggtagcaactgcaacagtagcaacgaaggctgtgggtctgcagctgtgttcccagtcgacattgctcacggtgaaggtgacatctccctccagtttcacaaaatgagtgtggtatggttacgaagaccaggacctaaacttcacagagagcccagcctgctgttctgatgccgaaagggagacagaaagtcccacgcttggatgcccgcctgggcctgcctatctgcctctgtctggaattagccatcttctttctggttccccaggtcatgggcctaacagaggcaggtggtcttgacaccttgggtgcaggggggctaagctgggctgtacatctggacagcctagaaggtgagaggaaggaagagagtctgatacaacaggcaaatgctgtggcccaggcagcggggctggtgaatgctgggcgcattggagagctccaggggcactacctctttgtccagccagctgggcacgggcaagccatggaggcggaggccatgcggcaacaggcagaggctgtgttagccaagcatgaagctgtgcgctggcactcagagcagaggctgctgaagagggccaagcgcagcatccacttcaatgatcccaagtatcctcaacagtggcacctgaataatcggcggagcccaggaagagacatcaatgtgacaggtgtgtgggagagaaatgtaactgggcgaggggtgacggtggtagtggtggacgacggagtggagcacaccgtccaggacattgcacccaactatagcccagagggaagctatgacctcaactctaatgacccagatcctatgccccaccctgatgaggagaacggtaaccaccatgggacccggtgtgcaggagaaattgcagctgtgcccaacaacagcttctgtgcagtaggtgtggcctatgggagccgaatagcaggtatccgggtgctggatggaccactcacagacagtatggaagctgtggcattcaacaaacactatcagatcaatgacatctacagctgcagctggggccccgatgatgatgggaagacagtggatggtcctcatcagcttgggaaggctgccttacaacatggagtgatggctggtcgccaaggctttggaagtatctttgtggttgccagtggtaatggtggccagcacaatgacaactgcaactatgatggctatgccaactccatctacactgtcaccataggagctgtggatgaggagggacggatgcctttttatgcagaggagtgtgcctccatgctggcagtcaccttcagtggaggagacaagatgctgcggagcattgtgactactgactgggaccttcagaagggcactggctgcactgaaggccacacaggaacctcagctgcagcccctctagcagctggcatgatagctctcatgctgcaggtgcggccctgcctcacgtggcgggatgtccagcacatcattgtcttcacagccactcagtatgaagatcatcgtgcagactggctcactaatgaggctggattcagccacagccatcagcatggtttcggcctgctcaacgcctggagacttgtcaatgcagccaagatctggacgtctgtcccttacttagcttcctatgtcagccccatgctgaaagaaaataaggctgttccacggtccccccactctctggaggtcctatggaatgtcagcaggacggacctggagatgtcggggctgaagaccctggaacatgtggcggtgacagtctccatcactcacccacgacgtggcagcttagaactgaaactgttttgtcccagtggcatgatgtctttgatcggcgcgccccgcagcatggactcggaccctaacggcttcaatgactggacattttccactgtgcggtgctggggggaaagagcaagaggagtctacagactggttatcagggatgtaggagatgagccgctccaggtgggcatcctccagcagtggcagctgacgctgtatggctccacgtggagtccagtagacatcaaggacagacaaagtctcttagaaagtgctatgagtggaaaatacctgcatgatgacttcactctgccttgcccacctggactgaaaattcctgaggaggatggttacagcattacccctaacacactcaagacccttgtgctggttggctgcttcagtgtcttctggaccatttactacatgctagaagtgtgcctgagccagaggagcaaggcttccacccatggctgcaggagaggatgctgcccctggcccccacaaagccaaaactccaaggaagtggggacagcactagaatcaatgccactgtgcagcagcaaggacctggatggagtggattcagagcacggggactgcacaactgcctctagcttgctggccccagagctgctgggggaagctgactggagtctgtcccagaacagtaagagtgacctggattgtcctccccaccaacccccagacctgaaggatggacagatctgctgaccccagagcccagcttcttccatgtacaacaggctcttcctaaacttggttatgaggctttcaatcatggatgctcaggagagaatggtcctgatagtgacatctgccacccaaggcctctgaagcattctcagccttctcaagagggtgagggccatcttaattaagcgcagtggtaagggactggtgttcatggctcctctagctgagctttgctgagagcctttcgacaaggggttggcgcccaggccaggcagcccttgatttcatctctctcgggctagtcactgtcctcggccatcaggacctccaccccttcacaagttatgcagcaggtgcctgtatacctaggaacactctcaacagaagtgccgctgtaccgagggcccagagagaggctggcaagagcctagacatgcctaccctgaaagcagctgccttcattaccctttcctgtgtgcctgttctgaagcccgtaccttcactaccactcacccttcctgctgaaggaatggtggcgtgtcccaggaaaactgggaggcacaggattcccagccagcacccttgctgcctcactggggaagttgtcctgctgggctgcagagagctgagacacagtgttgtctaagatagcatgggagcccctgcctatgaccacctgtcttcctctgcaaagtgctcagggaaatggccttcattccagaggccagctgtccgcctgacttttcctccattcttggccttctccccttcccatctttggagctaattgttaatatgaattttttaatgcttaagatttgatttttacttttcaaagcaacattttgttgaatttttttctgcacagctttccaaaataaaaagcagaagtaaaaaaa

SEQ ID NO: 7 - protein, 770 aa, Mus musculus
MPKGRQKVPHLDAHLGLPICLWLELAIFFLVPQVMGLSEAGGLDILGTGGLSWAVHLDSLEGERKEESLTQQADAVAQAAGLVNAGRIGELQGHYLFVQPTGHRQAMEVEAMRQQAEAVLARHEAVRWHSEQTLLKRAKRSIHFNDPKYPQQWHLNNRRSPGRDINVTGVWERNVTGRGVTVVVVDDGVEHTVQDIAPNYSPEGSYDLNSNDPDPMPHPDEENGNHHGTRCAGEIAAVPNNSFCAVGVAYGSRIAGIRVLDGPLTDSMEAVAFNKHYQINDIYSCSWGPDDDGKTVDGPHQLGKAALQHGVMAGRQGFGSIFVVASGNGGQHNDNCNYDGYANSIYTVTIGAVDEEGRMPFYAEECASMLAVTFSGGDKMLRSIVTTDWDLQKGTGCTEGHTGTSAAAPLAAGMIALMLQVRPCLTWRDVQHIIVFTAIQYEDHHADWLTNEAGFSHSHQHGFGLLNAWRLVNAAKIWTSVPYLASYVSPMLKENKAVPRSPHSLEVLWNVSRTDLEMSGLKTLEHVAVTVSITHPRRGSLELKLFCPSGMMSLIGAPRSMDSDPNGFNAWTFSTVRCWGERARGVYRLVIRDVGDEPLQMGILQQWQLTLYGSMWSPVDIKDRQSLLESAMSGKYLHDGFTLPCPPGLKIPEEDGYTITPNTLKTLVLVGCFSVFWTIYYMLEVCLSQRNKASTHGCRKGCCPWAPRRQNSKDAGTALESMPLCSSKDLDGVDSEHGDCTTASSFLAPELDCPPHQPPDLLQGKSGQIC

SEQ ID NO: 8 - RNA, 4098 nt, Mus musculus
  source 1..4098: mol_type = mRNA
agccctgagacccaagcggagcccgagggacacgcgggccagagagacggcggccggatggaggcgagagcctctctgaggagtttatggggaagagtcaaggctatggcagtccgacggagaagagccgcgaggggtgtttagaggtcggaaccgggatggtccttacagaggacgccatgaaggacgagggccctgtggcggagggggtcctgtggcggaggacacgaggattcgaggcgccggaactcccacagccctcacgggcctgtgacagaagctgtcggcggagcgcccggagctcggcccgctggtcaagaggccgcaggcccagccgctgattggctgcggcgccgggcggaagtgatgtcgctgtcagtggcgcctccggctccggggaagccgagagtttccgtagggtgggggctgcggcggcagcagcagcagcgacagtagcaacagaggctgtgggtgagtcgggaagaaatgtagtggaggctggacctcggatatacgggggaccatggaccggggctggtcggcgtctgcggaggaggtccttcgctgagggcaccttgggtcctggcgtgggcgaccccagagggaccgggtctccgggtcgtaggttccggacagcatcggtcctaaggagagagaggcacattgcacggagcagctgagtccgcgtgggagtgggggcatcagaaaactcactctgtacagaccctggttcctgtctgcagctgtgttcccagtcgacactcactgctcacagtgaaggtgacatctccctccattttcacaagatgagtgtggtatggttacccaggccaggacctaaacttcacagagagcccagcctgctgttctgatgccgaaagggaggcagaaagtcccacacttggatgcccacctgggcctgcccatctgcctctggctggaattagccatcttctttctggttccccaggtcatgggcctatcagaggcaggtgggcttgacatcttgggcacaggggggctgagctgggccgtacatctggacagcctagaaggtgagaggaaggaagagagtctgacgcaacaggccgatgctgtggcccaggcagcagggcttgtgaatgctgggcgcattggagaactccaggggcactacctctttgtccagcctactgggcataggcaagccatggaggtggaggccatgcggcaacaggcagaggctgtgttagccaggcatgaagctgtgcgctggcactcggagcaaacgctgctgaagagggccaagcgcagtatccacttcaatgatcccaagtaccctcaacagtggcacctgaataatcggcggagcccaggaagagacatcaatgtgacaggtgtgtgggagcgaaatgtaactgggcgtggggtgacagtggtggtggtggatgacggagtggaacacaccgtccaggacatcgcacccaactacagtccagagggaagttatgacctcaactctaatgacccagatcccatgccccaccctgatgaggagaatggtaaccaccatgggacccggtgtgcaggagaaattgcagctgtgcccaacaacagcttctgtgcagtgggtgtggcctacgggagccgaatagcaggtatccgggtgctggatggaccacttactgacagtatggaggctgtggcgttcaacaagcactatcagatcaatgacatctacagctgcagctggggcccagatgatgatgggaagacagtggatggtcctcatcagctcggaaaggctgccttacaacatggagtgatggctggtcgccaaggctttggaagtatctttgtggttgccagtggtaatgggggccagcacaatgacaactgcaactatgatggctatgccaactccatctacactgtcaccataggagctgtggatgaggagggacggatgcctttttatgcagaggagtgtgcctccatgctggcagtcaccttcagtggtggagacaagatgctgcggagcattgtgaccactgactgggaccttcagaagggcactggctgcactgaaggccacacaggaacctcagctgcagctcctctggcagctggaatgatagccctcatgctgcaggtgcggccctgcctcacatggcgggatgtacagcacataattgtcttcacagccatccagtatgaagatcatcatgcagactggctcaccaatgaggctggcttcagccacagccatcagcatggtttcggcctgctcaacgcttggagacttgtcaatgcagccaagatctggacatctgtcccttacttagcctcctatgtcagccccatgctgaaagaaaataaggctgttccgaggtccccccactctctggaggtgctatggaatgtcagcaggacggacctggagatgtcggggctgaagaccttggaacatgtggcagtgacagtctccatcactcacccacgacgtggcagcttagaactgaaactgttttgtcccagtggcatgatgtctttgatcggcgcgccccgcagcatggactcggatcctaatggcttcaatgcttggaccttttccactgtgcggtgctggggggaaagagcaagaggcgtctacagactggttatcagggatgtaggagatgagccgctccagatgggcatcctccagcagtggcagctgaccctgtatggctccatgtggagtccagtagacatcaaggacagacaaagtctcttagaaagtgctatgagtggaaaatacctgcatgatggcttcaccctgccttgcccacctgggctgaaaatccctgaggaggatggctacaccattacccctaacacactcaagacccttgtgctggtaggctgcttcagtgtcttctggactatttactacatgctagaagtgtgcctgagccagaggaacaaggcttccacccatggctgcaggaagggatgctgcccctgggccccacgaaggcaaaactccaaggatgcagggacagcactagaatcaatgccactgtgcagcagcaaggacctggatggagtggattcagagcatggggactgcacaactgcatctagcttcctggccccagagctggattgtcctccacaccaacccccagacctgctgcaggggaagagcgggcagatttgctgaccccagagcccagcttcccccatgtacaacaggcacttcctaaactcggttatgaggctttcaatcgtggaagctcaggagggaatggtcctgatagtgacatctgggaccccaggcctctgaagcatttgcagcctcctcaggagggtgagggccatcttaatgcagtggtaagggactggtgatcatggcttctctagctgaacttggctgagaacctttgacaagggattggcaccaagaccaggcagcccttgatttcctcgctctctggagctagtcagtgtcctagaccaccaggacctctactcattcatgagttagttatgcagcaagtccctatatacctagaaatgctcttgtcagaagtgtcactgccaagggaccagagagaggctgctaagagcctggacatgcctaccctgaaagcagctgccttcattacccttttctgtgcgcctgttctgaagccaacttaaacctgtccacatcttcactaccattcgccctttctgctgaaggaatggtgacgtgttcccaggagaactgggaggcacaggattcccagcacccttgctgcctcattggggaagttggccagctggacttcagagagctaagacacagcattttcaaagatagcctgggagcactgtctctgaccacctgtcttcctctgcaccgtgctcagggaaatggccttcattccagaggccagctgtctgcctgacttttcctccgttcttggccttctccccttcccatcttctgagctaattgttaatatgaattttttaatgcttaagatttgatttttacttttcaaagcaacattttgttgaatttttttctgcacagctttccaaaataaaaagcagaagtataaaaacccaaaaaaaaaaaaaaaaaaaaaa

SEQ ID NO: 9 - RNA, 3876 nt, Mus musculus
  source 1..3876: mol_type = mRNA
cggccgccaccgacggcgccctctggcggcaggcgcggacagcgaccggacaacggagccgctgaggggaccagccctgagacccaagcggagcccgagggacacgcgggccagagagacggcggccggatggaggcgagagcctctctgaggagtttatggggaagagtcaaggctatggcagtccgacggagaagagccgcgaggggtgtttagaggtcggaaccgggatggtccttacagaggacgccatgaaggacgagggccctgtggcggagggggtcctgtggcggaggacacgaggattcgaggcgccggaactcccacagccctcacgggcctgtgacagaagctgtcggcggagcgcccggagctcggcccgctggtcaagaggccgcaggcccagccgctgattggctgcggcgccgggcggaagtgatgtcgctgtcagtggcgcctccggctccggggaagccgagagtttccgtagggtgggggctgcggcggcagcagcagcagcgacagtagcaacagaggctgtgggtctgcagctgtgttcccagtcgacactcactgctcacagtgaaggtgacatctccctccattttcacaagatgagtgtggtatggttacccaggccaggacctaaacttcacagagagcccagcctgctgttctgatgccgaaagggaggcagaaagtcccacacttggatgcccacctgggcctgcccatctgcctctggctggaattagccatcttctttctggttccccaggtcatgggcctatcagaggcaggtgggcttgacatcttgggcacaggggggctgagctgggccgtacatctggacagcctagaaggtgagaggaaggaagagagtctgacgcaacaggccgatgctgtggcccaggcagcagggcttgtgaatgctgggcgcattggagaactccaggggcactacctctttgtccagcctactgggcataggcaagccatggaggtggaggccatgcggcaacaggcagaggctgtgttagccaggcatgaagctgtgcgctggcactcggagcaaacgctgctgaagagggccaagcgcagtatccacttcaatgatcccaagtaccctcaacagtggcacctgaataatcggcggagcccaggaagagacatcaatgtgacaggtgtgtgggagcgaaatgtaactgggcgtggggtgacagtggtggtggtggatgacggagtggaacacaccgtccaggacatcgcacccaactacagtccagagggaagttatgacctcaactctaatgacccagatcccatgccccaccctgatgaggagaatggtaaccaccatgggacccggtgtgcaggagaaattgcagctgtgcccaacaacagcttctgtgcagtgggtgtggcctacgggagccgaatagcaggtatccgggtgctggatggaccacttactgacagtatggaggctgtggcgttcaacaagcactatcagatcaatgacatctacagctgcagctggggcccagatgatgatgggaagacagtggatggtcctcatcagctcggaaaggctgccttacaacatggagtgatggctggtcgccaaggctttggaagtatctttgtggttgccagtggtaatgggggccagcacaatgacaactgcaactatgatggctatgccaactccatctacactgtcaccataggagctgtggatgaggagggacggatgcctttttatgcagaggagtgtgcctccatgctggcagtcaccttcagtggtggagacaagatgctgcggagcattgtgaccactgactgggaccttcagaagggcactggctgcactgaaggccacacaggaacctcagctgcagctcctctggcagctggaatgatagccctcatgctgcaggtgcggccctgcctcacatggcgggatgtacagcacataattgtcttcacagccatccagtatgaagatcatcatgcagactggctcaccaatgaggctggcttcagccacagccatcagcatggtttcggcctgctcaacgcttggagacttgtcaatgcagccaagatctggacatctgtcccttacttagcctcctatgtcagccccatgctgaaagaaaataaggctgttccgaggtccccccactctctggaggtgctatggaatgtcagcaggacggacctggagatgtcggggctgaagaccttggaacatgtggcagtgacagtctccatcactcacccacgacgtggcagcttagaactgaaactgttttgtcccagtggcatgatgtctttgatcggcgcgccccgcagcatggactcggatcctaatggcttcaatgcttggaccttttccactgtgcggtgctggggggaaagagcaagaggcgtctacagactggttatcagggatgtaggagatgagccgctccagatgggcatcctccagcagtggcagctgaccctgtatggctccatgtggagtccagtagacatcaaggacagacaaagtctcttagaaagtgctatgagtggaaaatacctgcatgatggcttcaccctgccttgcccacctgggctgaaaatccctgaggaggatggctacaccattacccctaacacactcaagacccttgtgctggtaggctgcttcagtgtcttctggactatttactacatgctagaagtgtgcctgagccagaggaacaaggcttccacccatggctgcaggaagggatgctgcccctgggccccacgaaggcaaaactccaaggatgcagggacagcactagaatcaatgccactgtgcagcagcaaggacctggatggagtggattcagagcatggggactgcacaactgcatctagcttcctggccccagagctggattgtcctccacaccaacccccagacctgctgcaggggaagagcgggcagatttgctgaccccagagcccagcttcccccatgtacaacaggcacttcctaaactcggttatgaggctttcaatcgtggaagctcaggagggaatggtcctgatagtgacatctgggaccccaggcctctgaagcatttgcagcctcctcaggagggtgagggccatcttaatgcagtggtaagggactggtgatcatggcttctctagctgaacttggctgagaacctttgacaagggattggcaccaagaccaggcagcccttgatttcctcgctctctggagctagtcagtgtcctagaccaccaggacctctactcattcatgagttagttatgcagcaagtccctatatacctagaaatgctcttgtcagaagtgtcactgccaagggaccagagagaggctgctaagagcctggacatgcctaccctgaaagcagctgccttcattacccttttctgtgcgcctgttctgaagccaacttaaacctgtccacatcttcactaccattcgccctttctgctgaaggaatggtgacgtgttcccaggagaactgggaggcacaggattcccagcacccttgctgcctcattggggaagttggccagctggacttcagagagctaagacacagcattttcaaagatagcctgggagcactgtctctgaccacctgtcttcctctgcaccgtgctcagggaaatggccttcattccagaggccagctgtctgcctgacttttcctccgttcttggccttctccccttcccatcttctgagctaattgttaatatgaattttttaatgcttaagatttgatttttacttttcaaagcaacattttgttgaatttttttctgcacagctttccaaaataaaaagcagaagtat

SEQ ID NO: 10 - RNA, 3893 nt, Mus musculus
  source 1..3893: mol_type = mRNA
agccctgagacccaagcggagcccgagggacacgcgggccagagagacggcggccggatggaggcgagagcctctctgaggagtttatggggaagagtcaaggctatggcagtccgacggagaagagccgcgaggggtgtttagaggtcggaaccgggatggtccttacagaggacgccatgaaggacgagggccctgtggcggagggggtcctgtggcggaggacacgaggattcgaggcgccggaactcccacagccctcacgggcctgtgacagaagctgtcggcggagcgcccggagctcggcccgctggtcaagaggccgcaggcccagccgctgattggctgcggcgccgggcggaagtgatgtcgctgtcagtggcgcctccggctccggggaagccgagagtttccgtagggtgggggctgcggcggcagcagcagcagcgacagtagcaacagaggctgtgggtctgcagctgtgttcccagtcgacactcactgctcacagtgaaggtgacatctccctccattttcacaagatgagtgtggtatggttacccaggccaggacctaaacttcacagagagcccagcctgctgttctgatgccgaaagggaggcagaaagtcccacacttggatgcccacctgggcctgcccatctgcctctggctggaattagccatcttctttctggttccccaggtcatgggcctatcagaggcaggtgggcttgacatcttgggcacaggggggctgagctgggccgtacatctggacagcctagaaggtgagaggaaggaagagagtctgacgcaacaggccgatgctgtggcccaggcagcagggcttgtgaatgctgggcgcattggagaactccaggggcactacctctttgtccagcctactgggcataggcaagccatggaggtggaggccatgcggcaacaggcagaggctgtgttagccaggcatgaagctgtgcgctggcactcggagcaaacgctgctgaagagggccaagcgcagtatccacttcaatgatcccaagtaccctcaacagtggcacctgaataatcggcggagcccaggaagagacatcaatgtgacaggtgtgtgggagcgaaatgtaactgggcgtggggtgacagtggtggtggtggatgacggagtggaacacaccgtccaggacatcgcacccaactacagtccagagggaagttatgacctcaactctaatgacccagatcccatgccccaccctgatgaggagaatggtaaccaccatgggacccggtgtgcaggagaaattgcagctgtgcccaacaacagcttctgtgcagtgggtgtggcctacgggagccgaatagcaggtatccgggtgctggatggaccacttactgacagtatggaggctgtggcgttcaacaagcactatcagatcaatgacatctacagctgcagctggggcccagatgatgatgggaagacagtggatggtcctcatcagctcggaaaggctgccttacaacatggagtgatggctggtcgccaaggctttggaagtatctttgtggttgccagtggtaatgggggccagcacaatgacaactgcaactatgatggctatgccaactccatctacactgtcaccataggagctgtggatgaggagggacggatgcctttttatgcagaggagtgtgcctccatgctggcagtcaccttcagtggtggagacaagatgctgcggagcattgtgaccactgactgggaccttcagaagggcactggctgcactgaaggccacacaggaacctcagctgcagctcctctggcagctggaatgatagccctcatgctgcaggtgcggccctgcctcacatggcgggatgtacagcacataattgtcttcacagccatccagtatgaagatcatcatgcagactggctcaccaatgaggctggcttcagccacagccatcagcatggtttcggcctgctcaacgcttggagacttgtcaatgcagccaagatctggacatctgtcccttacttagcctcctatgtcagccccatgctgaaagaaaataaggctgttccgaggtccccccactctctggaggtgctatggaatggacggacctggagatgtcggggctgaagaccttggaacatgtggcagtgacagtctccatcactcacccacgacgtggcagcttagaactgaaactgttttgtcccagtggcatgatgtctttgatcggcgcgccccgcagcatggactcggatcctaatggcttcaatgcttggaccttttccactgtgcggtgctggggggaaagagcaagaggcgtctacagactggttatcagggatgtaggatagcaacaaaaattccttgttgaaaccacatttcaagccctttcacatataaccatccttcggccagtcctgataggcaggagatgagccgctccagatgggcatcctccagcagtggcagctgaccctgtatggctccatgtggagtccagtagacatcaaggacagacaaagtctcttagaaagtgctatgagtggaaaatacctgcatgatggcttcaccctgccttgcccacctgggctgaaaatccctgaggaggatggctacaccattacccctaacacactcaagacccttgtgctggtaggctgcttcagtgtcttctggactatttactacatgctagaagtgtgcctgagccagaggaacaaggcttccacccatggctgcaggaagggatgctgcccctgggccccacgaaggcaaaactccaaggatgcagggacagcactagaatcaatgccactgtgcagcagcaaggacctggatggagtggattcagagcatggggactgcacaactgcatctagcttcctggccccagagctggattgtcctccacaccaacccccagacctgctgcaggggaagagcgggcagatttgctgaccccagagcccagcttcccccatgtacaacaggcacttcctaaactcggttatgaggctttcaatcgtggaagctcaggagggaatggtcctgatagtgacatctgggaccccaggcctctgaagcatttgcagcctcctcaggagggtgagggccatcttaatgcagtggtaagggactggtgatcatggcttctctagctgaacttggctgagaacctttgacaagggattggcaccaagaccaggcagcccttgatttcctcgctctctggagctagtcagtgtcctagaccaccaggacctctactcattcatgagttagttatgcagcaagtccctatatacctagaaatgctcttgtcagaagtgtcactgccaagggaccagagagaggctgctaagagcctggacatgcctaccctgaaagcagctgccttcattacccttttctgtgcgcctgttctgaagccaacttaaacctgtccacatcttcactaccattcgccctttctgctgaaggaatggtgacgtgttcccaggagaactgggaggcacaggattcccagcacccttgctgcctcattggggaagttggccagctggacttcagagagctaagacacagcattttcaaagatagcctgggagcactgtctctgaccacctgtcttcctctgcaccgtgctcagggaaatggccttcattccagaggccagctgtctgcctgacttttcctccgttcttggccttctccccttcccatcttctgagctaattgttaatatgaattttttaatgcttaagatttgatttttacttttcaaagcaacattttgttgaatttttttctgcacagctttccaaaataaaaagcagaagtataaaaacccaaacaa

SEQ ID NO: 11 - RNA, 3599 nt, Mus musculus
  source 1..3599: mol_type = mRNA
agggtgggggctgcggcggcagcagcagcagcgacagtagcaacagaggctgtgggtgagtcgggaagaaatgtagtggaggctggacctcggatatacgggggaccatggaccggggctggtcggcgtctgcggaggaggtccttcgctgagggcaccttgggtcctggcgtgggcgaccccagagggaccgggtctccgggtcgtaggttccggacagcatcggtcctaaggagagagaggcacattgcacggagcagctgagtccgcgtgggagtgggggcatcagaaaactcactctgtacagaccctggttcctgtctgcagctgtgttcccagtcgacactcactgctcacagtgaaggtgacatctccctccattttcacaagatgagtgtggtatggttacccaggccaggacctaaacttcacagagagcccagcctgctgttctgatgccgaaagggaggcagaaagtcccacacttggatgcccacctgggcctgcccatctgcctctggctggaattagccatcttctttctggttccccaggtcatgggcctatcagaggcaggtgggcttgacatcttgggcacaggggggctgagctgggccgtacatctggacagcctagaaggtgagaggaaggaagagagtctgacgcaacaggccgatgctgtggcccaggcagcagggcttgtgaatgctgggcgcattggagaactccaggggcactacctctttgtccagcctactgggcataggcaagccatggaggtggaggccatgcggcaacaggcagaggctgtgttagccaggcatgaagctgtgcgctggcactcggagcaaacgctgctgaagagggccaagcgcagtatccacttcaatgatcccaagtaccctcaacagtggcacctgaataatcggcggagcccaggaagagacatcaatgtgacaggtgtgtgggagcgaaatgtaactgggcgtggggtgacagtggtggtggtggatgacggagtggaacacaccgtccaggacatcgcacccaactacagtccagagggaagttatgacctcaactctaatgacccagatcccatgccccaccctgatgaggagaatggtaaccaccatgggacccggtgtgcaggagaaattgcagctgtgcccaacaacagcttctgtgcagtgggtgtggcctacgggagccgaatagcaggtatccgggtgctggatggaccacttactgacagtatggaggctgtggcgttcaacaagcactatcagatcaatgacatctacagctgcagctggggcccagatgatgatgggaagacagtggatggtcctcatcagctcggaaaggctgccttacaacatggagtgatggctggtcgccaaggctttggaagtatctttgtggttgccagtggtaatgggggccagcacaatgacaactgcaactatgatggctatgccaactccatctacactgtcaccataggagctgtggatgaggagggacggatgcctttttatgcagaggagtgtgcctccatgctggcagtcaccttcagtggtggagacaagatgctgcggagcattgtgaccactgactgggaccttcagaagggcactggctgcactgaaggccacacaggaacctcagctgcagctcctctggcagctggaatgatagccctcatgctgcaggtgcggccctgcctcacatggcgggatgtacagcacataattgtcttcacagccatccagtatgaagatcatcatgcagactggctcaccaatgaggctggcttcagccacagccatcagcatggtttcggcctgctcaacgcttggagacttgtcaatgcagccaagatctggacatctgtcccttacttagcctcctatgtcagccccatgctgaaagaaaataaggctgttccgaggtccccccactctctggaggtgctatggaatgtcagcaggacggacctggagatgtcggggctgaagaccttggaacatgtggcagtgacagtctccatcactcacccacgacgtggcagcttagaactgaaactgttttgtcccagtggcatgatgtctttgatcggcgcgccccgcagcatggactcggatcctaatggcttcaatgcttggaccttttccactgtgcggtgctggggggaaagagcaagaggcgtctacagactggttatcagggatgtaggagatgagccgctccagatgggcatcctccagcagtggcagctgaccctgtatggctccatgtggagtccagtagacatcaaggacagacaaagtctcttagaaagtgctatgagtggaaaatacctgcatgatggcttcaccctgccttgcccacctgggctgaaaatccctgaggaggatggctacaccattacccctaacacactcaagacccttgtgctggtaggctgcttcagtgtcttctggactatttactacatgctagaagtgtgcctgagccagaggaacaaggcttccacccatggctgcaggaagggatgctgcccctgggccccacgaaggcaaaactccaaggatgcagggacagcactagaatcaatgccactgtgcagcagcaaggacctggatggagtggattcagagcatggggactgcacaactgcatctagcttcctggccccagagctggattgtcctccacaccaacccccagacctgctgcaggggaagagcgggcagatttgctgaccccagagcccagcttcccccatgtacaacaggcacttcctaaactcggttatgaggctttcaatcgtggaagctcaggagggaatggtcctgatagtgacatctgggaccccaggcctctgaagcatttgcagcctcctcaggagggtgagggccatcttaatgcagtggtaagggactggtgatcatggcttctctagctgaacttggctgagaacctttgacaagggattggcaccaagaccaggcagcccttgatttcctcgctctctggagctagtcagtgtcctagaccaccaggacctctactcattcatgagttagttatgcagcaagtccctatatacctagaaatgctcttgtcagaagtgtcactgccaagggaccagagagaggctgctaagagcctggacatgcctaccctgaaagcagctgccttcattacccttttctgtgcgcctgttctgaagccaacttaaacctgtccacatcttcactaccattcgccctttctgctgaaggaatggtgacgtgttcccaggagaactgggaggcacaggattcccagcacccttgctgcctcattggggaagttggccagctggacttcagagagctaagacacagcattttcaaagatagcctgggagcactgtctctgaccacctgtcttcctctgcaccgtgctcagggaaatggccttcattccagaggccagctgtctgcctgacttttcctccgttcttggccttctccccttcccatcttctgagctaattgttaatatgaattttttaatgcttaagatttgatttttacttttcaaagcaa

SEQ ID NO: 12 - RNA, 19 nt, Synthetic construct
  source 1..19: mol_type = other RNA
gaaaatacctgcacgatga

SEQ ID NO: 13 - RNA, 19 nt, Synthetic construct
  source 1..19: mol_type = other RNA
ctacgtcagtcccgtgtta

SEQ ID NO: 14 - RNA, 19 nt, Synthetic construct
  source 1..19: mol_type = other RNA
ggacattgcacccaactat

SEQ ID NO: 15 - RNA, 19 nt, Synthetic construct
  source 1..19: mol_type = other RNA
cagcaaggatccagacgaa

SEQ ID NO: 16 - RNA, 23 nt, Homo sapiens
  source 1..23: mol_type = mRNA
tgggggagggccgggctcagggc

SEQ ID NO: 17 - RNA, 23 nt, Homo sapiens
  source 1..23: mol_type = mRNA
cttcctctgcaagtgctcaggga

SEQ ID NO: 18 - RNA, 23 nt, Homo sapiens
  source 1..23: mol_type = mRNA
gtttggcctccatcctcatctct

SEQ ID NO: 19 - RNA, 23 nt, Homo sapiens
  source 1..23: mol_type = mRNA
ccatagccaggagcgcatctcaa

SEQ ID NO: 20 - RNA, 23 nt, Homo sapiens
  source 1..23: mol_type = mRNA
tttctcttttcacagcaacattt

SEQ ID NO: 21 - RNA, 23 nt, Homo sapiens
  source 1..23: mol_type = mRNA
gaatttttttctgcacagctttt

SEQ ID NO: 22 - RNA, 24 nt, Synthetic construct
  source 1..24: mol_type = other RNA
tccctgagaccctttaacctgtga

SEQ ID NO: 23 - RNA, 21 nt, Synthetic construct
  source 1..21: mol_type = other RNA
tgagatgaagcactgtagctc

SEQ ID NO: 24 - RNA, 22 nt, Synthetic construct
  source 1..22: mol_type = other RNA
gaatgttgctcggtgaacccct

SEQ ID NO: 25 - RNA, 22 nt, Synthetic construct
  source 1..22: mol_type = other RNA
aaaagctgggttgagagggcga

SEQ ID NO: 26 - RNA, 2355 nt, Homo sapiens
  source 1..2355: mol_type = mRNA
atgccgaaggggaggcagaaagtgccacacttggatgcccccctgggcctgcccacctgcctctggctggaattagccgggctcttcttactggttccctgggtcatgggcctggcagggacaggtgggcctgatggccagggcacaggggggccgagctgggctgtgcacctggaaagcctggaaggtgacggggaggaagagactctggagcagcaggcggatgccttggcccaggcagcagggctggtgaatgctggacgcatcggagagcttcaggggcactacctctttgtccagcctgctgggcacaggccggccctggaggtggaggccatccggcagcaggtggaggctgtgttggctgggcatgaagctgtgcgctggcactcagagcagaggctgctaaggcgggccaagcgcagcgtccacttcaacgaccccaagtacccgcagcaatggcacctgaataaccgacggagcccgggcagggacatcaacgtgacgggtgtgtgggaacgcaatgtgactgggcgaggggtgacggtggtggtagtggatgacggagtggaacacaccatccaggacattgcacccaactatagccctgagggtagctatgacctcaactctaatgaccctgaccccatgccccacccggatgtggagaatggcaaccaccatggcacgcgatgtgcaggagagatcgcggctgtgcccaacaacagcttctgtgccgtgggcgtggcctacgggagccgcatcgcaggtatccgggtactggatggacctctcacagacagcatggaggcagtggcgttcaacaagcactatcagatcaatgacatctacagctgcagctggggaccagatgacgatgggaagacagtggatggcccccatcagcttggaaaggctgccttacaacatggggtgattgctggtcgccagggctttgggagcatctttgtggtagccagtggcaacggaggccaacacaacgacaactgcaactacgatggctacgccaactccatctacaccgtcaccataggagctgtggatgaggagggacgcatgcctttctatgcagaagaatgtgcctccatgctggcagtcaccttcagtggtggggacaagatgcttcggagcattgtgaccactgactgggaccttcagaagggcactggctgcactgagggccacacagggacctcagctgcagcgcctctggcagctggcatgatagccttaatgctgcaggtgcggccctgcctcacgtggcgtgacgtccagcacatcattgtcttcacagccacccggtatgaggatcgccgtgcagagtgggtcaccaacgaggcaggcttcagccatagccaccagcacggtttcggcctcctcaacgcctggaggctcgtgaatgcagccaagatctggacatctgtcccttacttagcatcctacgtcagtcccgtgttaaaagaaaacaaggcgattccgcagtccccccgttccctggaggtcctgtggaatgtcagcaggatggacctggagatgtcagggctgaagaccctggagcatgtggcagtgacagtctccatcactcacccacggcgcggcagcttggagctgaagctgttctgccccagtggcatgatgtccctcatcggcgccccccgcagcatggactcggatcccaacggcttcaatgactggaccttctccactgtgcgatgctggggggagagagcccgagggacctacaggcttgtcatcagggatgtcggggatgagtcattccaggtcggcatcctccggcaatggcagctgaccctatatggctctgtgtggagtgcagtagacatcagggacagacaaaggctgttagagagtgccatgagtggaaaatacctgcacgatgacttcgccctgccctgcccaccggggctgaaaattcctgaggaagatggttacaccatcacccccaacaccctcaagaccctggtgctggtaggctgtttcaccgtcttctggactgtttactacatgctggaagtatatttgagccagaggaatgtggcttccaatcaagtttgtaggagtggaccctgccactggccccatcggagccggaaagccaaggaggaagggacagagctagaatcagtgccactttgcagcagcaaggatccagacgaagtggaaacagagagcaggggccctcccaccacctctgacctccttgccccagacctgctggagcaaggggactggagcctgtcccagaacaagagcgccctggactgccctcatcagcacctagacgtaccgcacgggaaggaggagcagatctgc

SEQ ID NO: 27 - RNA, 20 nt, Synthetic construct
  source 1..20: mol_type = other RNA
  modified_base 10: mod_base = OTHER; note = thymine
  modified_base 13: mod_base = OTHER; note = thymine
gacgtaggatgctaagtaag

SEQ ID NO: 28 - RNA, 20 nt, Synthetic construct
  source 1..20: mol_type = other RNA
  modified_base 7..8: mod_base = OTHER; note = thymine
  modified_base 13: mod_base = OTHER; note = thymine
atggagttggcgtagccatc

SEQ ID NO: 29 - RNA, 20 nt, Synthetic construct
  source 1..20: mol_type = other RNA
  modified_base 8: mod_base = OTHER; note = thymine
  modified_base 11: mod_base = OTHER; note = thymine
  modified_base 14..15: mod_base = OTHER; note = thymine
gatctgatagtgcttgttga

SEQ ID NO: 30 - RNA, 20 nt, Synthetic construct
  source 1..20: mol_type = other RNA
  modified_base 6..7: mod_base = OTHER; note = thymine
  modified_base 13: mod_base = OTHER; note = thymine
  modified_base 15: mod_base = OTHER; note = thymine
cagcattaaggctatcatgc

SEQ ID NO: 31 - RNA, 20 nt, Synthetic construct
  source 1..20: mol_type = other RNA
  modified_base 6: mod_base = OTHER; note = thymine
  modified_base 10: mod_base = OTHER; note = thymine
  modified_base 12: mod_base = OTHER; note = thymine
cactttcactgtgagtagtg

SEQ ID NO: 32 - RNA, 20 nt, Synthetic construct
  source 1..20: mol_type = other RNA
  modified_base 6: mod_base = OTHER; note = thymine
  modified_base 8..9: mod_base = OTHER; note = thymine
  modified_base 12: mod_base = OTHER; note = thymine
  modified_base 11: mod_base = OTHER; note = thymine
cacattcttctgcatagaaa

SEQ ID NO: 33 - RNA, 20 nt, Synthetic construct
  source 1..20: mol_type = other RNA
  modified_base 10: mod_base = OTHER; note = thymine
cccccagcatcgcacagtgg

SEQ ID NO: 34 - RNA, 20 nt, Synthetic construct
  source 1..20: mol_type = other RNA
  modified_base 7: mod_base = OTHER; note = thymine
  modified_base 9: mod_base = OTHER; note = thymine
  modified_base 15: mod_base = OTHER; note = thymine
tccaagtgtggcactttctg

SEQ ID NO: 35 - RNA, 20 nt, Synthetic construct
  source 1..20: mol_type = other RNA
  modified_base 10..11: mod_base = OTHER; note = thymine
  modified_base 14..15: mod_base = OTHER; note = thymine
acagtggcattgattctagt

SEQ ID NO: 36 - RNA, 20 nt, Synthetic construct
  source 1..20: mol_type = other RNA
  modified_base 6: mod_base = OTHER; note = thymine
  modified_base 9: mod_base = OTHER; note = thymine
  modified_base 11: mod_base = OTHER; note = thymine
  modified_base 15: mod_base = OTHER; note = thymine
ctccatactgtcagtaagtg

SEQ ID NO: 37 - RNA, 20 nt, Synthetic construct
  source 1..20: mol_type = other RNA
  modified_base 12: mod_base = OTHER; note = thymine
  modified_base 14: mod_base = OTHER; note = thymine
  modified_base 16: mod_base = OTHER; note = thymine
ctgataaccagtctgtagac

SEQ ID NO: 38 - RNA, 20 nt, Synthetic construct
  source 1..20: mol_type = other RNA
  modified_base 8..9: mod_base = OTHER; note = thymine
  modified_base 14..15: mod_base = OTHER; note = thymine
atcatagttgcagttgtcat

SEQ ID NO: 39 - RNA, 20 nt, Synthetic construct
  source 1..20: mol_type = other RNA
  modified_base 13: mod_base = OTHER; note = thymine
gacataggaggctaagtaag

SEQ ID NO: 40 - RNA, 20 nt, Synthetic construct
  source 1..20: mol_type = other RNA
  modified_base 7: mod_base = OTHER; note = thymine
  modified_base 12: mod_base = OTHER; note = thymine
tgagtgtcgactgggaacac

SEQ ID NO: 41 - RNA, 20 nt, Synthetic construct
  source 1..20: mol_type = other RNA
  modified_base 7: mod_base = OTHER; note = thymine
  modified_base 9..10: mod_base = OTHER; note = thymine
  modified_base 13: mod_base = OTHER; note = thymine
gatgtctcttcctgggctcc

SEQ ID NO: 42 - RNA, 20 nt, Synthetic construct
  source 1..20: mol_type = other RNA
  modified_base 9: mod_base = OTHER; note = thymine
  modified_base 12..13: mod_base = OTHER; note = thymine
gatctgggtcattagagttg

SEQ ID NO: 43 - RNA, 19 nt, Synthetic construct
  source 1..19: mol_type = other RNA
acgcatccctgagaccctt

SEQ ID NO: 44 - RNA, 19 nt, Synthetic construct
  source 1..19: mol_type = other RNA
cagtgcagggtccgaggta

SEQ ID NO: 45 - RNA, 50 nt, Synthetic construct
  source 1..50: mol_type = other RNA
gtcgtatccagtgcagggtccgaggtattcgcactggatacgactcacag

SEQ ID NO: 46 - RNA, 20 nt, Synthetic construct
  source 1..20: mol_type = other RNA
gcacgcatgagatgaagcac

SEQ ID NO: 47 - RNA, 18 nt, Synthetic construct
  source 1..18: mol_type = other RNA
agtgcagggtccgaggta

SEQ ID NO: 48 - RNA, 50 nt, Synthetic construct
  source 1..50: mol_type = other RNA
gtcgtatccagtgcagggtccgaggtattcgcactggatacgacgagcta

SEQ ID NO: 49 - RNA, 20 nt, Synthetic construct
  source 1..20: mol_type = other RNA
acgcaaaaagctgggttgag

SEQ ID NO: 50 - RNA, 18 nt, Synthetic construct
  source 1..18: mol_type = other RNA
agtgcagggtccgaggta

SEQ ID NO: 51 - RNA, 50 nt, Synthetic construct
  source 1..50: mol_type = other RNA
gtcgtatccagtgcagggtccgaggtattcgcactggatacgactcgccc

SEQ ID NO: 52 - RNA, 17 nt, Synthetic construct
  source 1..17: mol_type = other RNA
gggaatgttgctcggtg

SEQ ID NO: 53 - RNA, 19 nt, Synthetic construct
  source 1..19: mol_type = other RNA
cagtgcagggtccgaggta

SEQ ID NO: 54 - RNA, 39 nt, Synthetic construct
  source 1..39: mol_type = other RNA
gtcgtatccagtgcagggtccgaggtattcgcactggat

SEQ ID NO: 55 - RNA, 21 nt, Synthetic construct
  source 1..21: mol_type = other RNA
tgatgaccccaggtaactctg

SEQ ID NO: 56 - RNA, 18 nt, Synthetic construct
  source 1..18: mol_type = other RNA
agtgcagggtccgaggta

SEQ ID NO: 57 - RNA, 50 nt, Synthetic construct
  source 1..50: mol_type = other RNA
gtcgtatccagtgcagggtccgaggtattcgcactggatacgacggtcag

SEQ ID NO: 58 - RNA, 20 nt, Synthetic construct
  source 1..20: mol_type = other RNA
acaccatccaggacattgca

SEQ ID NO: 59 - RNA, 25 nt, Synthetic construct
  source 1..25: mol_type = other RNA
gggtcattagagttgaggtcatagc

SEQ ID NO: 60 - RNA, 22 nt, Synthetic construct
  source 1..22: mol_type = other RNA
gggtttcatccatccgacattg

SEQ ID NO: 61 - RNA, 21 nt, Synthetic construct
  source 1..21: mol_type = other RNA
tggttcacacggcaggcatac

SEQ ID NO: 62 - RNA, 19 nt, Synthetic construct
  source 1..19: mol_type = other RNA
atggggaaggtgaaggtcg

SEQ ID NO: 63 - RNA, 23 nt, Synthetic construct
  source 1..23: mol_type = other RNA
ggggtcattgatggcaaccaata

SEQ ID NO: 64 - RNA, 27 nt, Synthetic construct
  source 1..27: mol_type = other RNA
ccctcgagcctcagggcctgacagtgt

SEQ ID NO: 65 - RNA, 35 nt, Synthetic construct
  source 1..35: mol_type = other RNA
ttgcggccgcattgtttggaaggtttttattttgg

SEQ ID NO: 66 - RNA, 18 nt, Synthetic construct
  source 1..18: mol_type = other RNA
ggtctttctgtctctggc

SEQ ID NO: 67 - RNA, 18 nt, Synthetic construct
  source 1..18: mol_type = other RNA
aggtttcagttggtggtc

SEQ ID NO: 68 - RNA, 28 nt, Synthetic construct
  source 1..28: mol_type = other RNA
cggaattcctaacaccccttctcctggc

SEQ ID NO: 69 - RNA, 28 nt, Synthetic construct
  source 1..28: mol_type = other RNA
cgggatccttccccagcatcacaagtgg

SEQ ID NO: 70 - RNA, 19 nt, Synthetic construct
  source 1..19: mol_type = other RNA
gtcttctgcaagcacagcc

SEQ ID NO: 71 - RNA, 23 nt, Synthetic construct
  source 1..23: mol_type = other RNA
caattactttccaactggtcgac

SEQ ID NO: 72 - RNA, 27 nt, Synthetic construct
  source 1..27: mol_type = other RNA
cggaattcggggcggaagtgacgttag

SEQ ID NO: 73 - RNA, 26 nt, Synthetic construct
  source 1..26: mol_type = other RNA
cgggatccaccctgatcttggcgcct

SEQ ID NO: 74 - RNA, 26 nt, Synthetic construct
  source 1..26: mol_type = other RNA
ccctcgagtgggacaggctcttcttt

SEQ ID NO: 75 - RNA, 38 nt, Synthetic construct
  source 1..38: mol_type = other RNA
agatagctggcctccggcggaagacagggtggtggcag

SEQ ID NO: 76 - RNA, 38 nt, Synthetic construct
  source 1..38: mol_type = other RNA
ctgccaccaccctgtcttccgccggaggccagctatct

SEQ ID NO: 77 - RNA, 37 nt, Synthetic construct
  source 1..37: mol_type = other RNA
ctaggcagctggcttggcaggccaaaccaaagggggg

SEQ ID NO: 78 - RNA, 37 nt, Synthetic construct
  source 1..37: mol_type = other RNA
cccccctttggtttggcctgccaagccagctgcctag

SEQ ID NO: 79 - RNA, 39 nt, Synthetic construct
  source 1..39: mol_type = other RNA
gttgagaaggtgcctgccaaaacatcactggggtcactt

SEQ ID NO: 80 - RNA, 39 nt, Synthetic construct
  source 1..39: mol_type = other RNA
aagtgaccccagtgatgttttggcaggcaccttctcaac

SEQ ID NO: 81 - RNA, 39 nt, Synthetic construct
  source 1..39: mol_type = other RNA
aagtgaccccagtgatgtttaggccaaaccaaagggggg

SEQ ID NO: 82 - RNA, 39 nt, Synthetic construct
  source 1..39: mol_type = other RNA
cccccctttggtttggcctaaacatcactggggtcactt

SEQ ID NO: 83 - RNA, 31 nt, Synthetic construct
  source 1..31: mol_type = other RNA
ttgcggccgcaccaactagctcagaagaggg

(19) United States
(12) Patent Application Publication
(10) Pub. No.: US 2025/0129370 A1
(43) Pub. Date: Apr. 24, 2025

(54) METHOD FOR REDUCING HEPATIC TRIGLYCERIDES

(71) Applicant: ADAERATA, LIMITED PARTNERSHIP, Quebec (CA)

(72) Inventors: Nabil G. SEIDAH, Verdun (CA); SAUVAGEAU Martin, Montreal (CA)

(21) Appl. No.: 18/835,788

(22) PCT Filed: Feb. 7, 2023
(86) PCT No.: PCT/CA2023/050155

§ 371 Date: Aug. 5, 2024

Related U.S. Application Data
(60) Provisional application No. 63/267,671, filed on Feb. 8, 2022.

(51) Int. Cl. C12N 15/113; A61K 45/06; A61P 1/16; A61P 3/06

(52) U.S. Cl. CPC C12N 15/1137; A61K 45/06; A61P 1/16; A61P 3/06; C12N 2310/11; C12N 2310/141; C12N 2310/315; C12N 2310/321; C12N 2310/341; C12Y 304/21062

(57) ABSTRACT

A method of reducing the level of hepatic triglycerides in a subject in need thereof comprising administering a therapeutically effective amount of a proprotein convertase 7 (PC7) inhibitor to the subject. Also provided herein are compositions and kits comprising said inhibitor.

### CROSS REFERENCE TO RELATED APPLICATIONS

This application is a PCT application Serial No PCT/CA2023/* filed on Feb. 7, 2023 and published in English under PCT Article 21(2), which itself claims benefit of U.S. provisional application Ser. No. 63/267,671, filed on Feb. 8, 2022. All documents above are incorporated herein in their entirety by reference.

### STATEMENT REGARDING FEDERALLY SPONSORED RESEARCH OR DEVELOPMENT

N.A.

### FIELD OF THE DISCLOSURE

The present disclosure relates to a method of reducing hepatic triglycerides. More specifically, the present disclosure is concerned with a method of reducing hepatic triglycerides with a proprotein convertase 7 (PC7) inhibitor.

### REFERENCE TO SEQUENCE LISTING

Pursuant to 37 C.F.R. 1.821(c), a sequence listing is submitted herewith as an ASCII compliant text file named Sequence listing_G12810-00836, that was created on Feb. 7, 2023 and having a size of 128 kilobytes. The content of the aforementioned file named Sequence listing_G12810-00836 is hereby incorporated by reference in its entirety.

### BACKGROUND OF THE DISCLOSURE

Nonalcoholic fatty liver disease (NAFLD) is a generic term encompassing multiple liver conditions affecting individuals who drink little to no alcohol. NAFLDs are characterized by excessive fat stored in liver cells. They are increasingly common around the world, and particularly in Western nations. It affects for example about 20% of the Canadian population and currently has no established treatments. Nonalcoholic steatohepatitis (NASH) is a type of NAFLD characterized by liver inflammation and damage caused by a buildup of fat in the liver (See FIG. 1 showing the progression from healthy liver to fatty liver and NASH liver). NASH is associated with high triglyceride levels.

There is a need for alternative methods of reducing hepatic triglyceride levels e.g., in NAFLD.

The present description refers to a number of documents, the content of which is herein incorporated by reference in their entirety.

### SUMMARY OF THE DISCLOSURE

More specifically, in accordance with the present disclosure, there is provided a method of reducing the level of hepatic triglycerides in a subject in need thereof comprising administering a therapeutically effective amount of a proprotein convertase 7 (PC7) inhibitor to the subject. In another specific embodiment, the present disclosure provides a method of treating NAFLD or a symptom thereof.

More specifically, in accordance with the present disclosure, there are provided the following items:

Item 1. A method of reducing the level of hepatic triglycerides in a subject in need thereof comprising administering a therapeutically effective amount of a proprotein convertase 7 (PC7) inhibitor to the subject.

Item 2. The method of item 1 wherein the inhibitor is an anti-PC7 antisense oligonucleotide (ASO), an anti-PC7 microRNA (miRNA), an anti-PC7 small interfering RNA (siRNA) or a CRISPR base editor or a prime editor.

Item 3. The method of item 2, wherein the inhibitor is an ASO.

Item 4. The method of item 3, wherein the ASO targets a human PC7 RNA exon or transcript.

Item 5. The method of item 2, wherein the inhibitor is a siRNA.

Item 6. The method of item 2, wherein the inhibitor is an miRNA.

Item 7. The method of item 6, wherein the miRNA targets one or more of human PCSK7 3′-UTR, PCSK7 exon 14, or PCSK7 exon 15.

Item 8. The method of any one of items 1-7, wherein the subject has a nonalcoholic fatty liver disease (NAFLD) or is a likely candidate for NAFLD.

Item 9. The method of item 8, wherein the subject has a nonalcoholic steatohepatitis (NASH) or is a likely candidate for NASH.

Item 10. The method of any one of items 1-9, wherein the subject is a human.

Item 11. A kit for reducing the level of hepatic triglycerides in a subject comprising:
(A) a proprotein convertase 7 (PC7) inhibitor; and
(B) (i) another agent for the prevention or the treatment of a nonalcoholic fatty liver disease (NAFLD) or a symptom thereof;

Item 12. (ii) a pharmaceutically acceptable carrier;

Item 13. (iii) instructions to use the kit for reducing the level of hepatic triglycerides; or Item 14. (iv) a combination of at least two of (i) to (iii).

Item 15. The kit of item 11, wherein the PC7 inhibitor is (a) an anti-PC7 antisense oligonucleotide (ASO), (b) an anti-PC7 microRNA (miRNA) against PC7; or (c) an anti-PC7 small interfering RNA (siRNA).

Item 16. The kit of item 12, wherein the ASO, miRNA or the siRNA is specific to human PC7.

Item 17. A composition comprising (A) a proprotein convertase 7 (PC7) inhibitor; and (B) (i) another agent for the prevention or the treatment of a nonalcoholic fatty liver disease (NAFLD) or a symptom thereof; (ii) a pharmaceutically acceptable carrier; or (iv) a combination of at least two of (i) to (iii).

Item 18. The composition of item 14, wherein the PC7 inhibitor is (a) an anti-PC7 antisense oligonucleotide (ASO) specific to PC7; (b) an anti-PC7 microRNA (miRNA); or (c) an anti-PC7 small interfering RNA (siRNA).

Item 19. The composition of item 15, wherein the ASO, miRNA or the siRNA is specific to human PC7.

Item 20. The composition of item 16, wherein the ASO targets a human PC7 RNA transcript.

Item 21. The composition of item 16, wherein the inhibitor is an siRNA.

Item 22. The method of item 16, wherein the inhibitor is an miRNA.

Item 23. The method of item 19, wherein the miRNA targets one or more of human PCSK7 3′-UTR, PCSK7 exon 14, or PCSK7 exon 15.

In another embodiment, there is provided an PC7 inhibitor as described herein such as but not limited to an anti-PC7 siRNA e.g., those illustrated FIG. 6A, an anti-PC7 ASO e.g., those illustrated in FIG. 33B or 35B, as optionally further modified to target hepatocytes. For example, ASOs are conjugated at their 5′ and/or 3′ terminus with a GalNAc moiety as described herein. In another embodiment, there is provided a composition comprising the PC7 inhibitor and a pharmaceutically acceptable excipient.

### Definitions

Terms and symbols of genetics, molecular biology, biochemistry and nucleic acid used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

The use of the terms “a” and “an” and “the” and similar referents in the context of describing the disclosure (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the disclosure and does not pose a limitation on the scope of the disclosure unless otherwise claimed.

No language in the specification should be construed as indicating any non-claimed element as essential to the practice of the disclosure.

Herein, the term “about” has its ordinary meaning. In embodiments, it may mean plus or minus 10% of the numerical value qualified.

Unless otherwise defined, all technical and scientific terms used herein have the same meaning as commonly understood by one of ordinary skill in the art to which this disclosure belongs.

### PC7 Inhibitors

As used herein the term “PC7” called “proprotein convertase 7” or “proprotein convertase subtilisin/kexin type 7 (PCSK7)” (EC 3.4.21. B27) belongs to the subtilisin-like proprotein convertase family, proprotein convertases that process latent precursor proteins into their biologically active products. It is a calcium-dependent serine endoprotease and is structurally related to its family members, Furin, PC5 and PACE4. It is concentrated in the trans-Golgi network, associated with the membranes, since it has a transmembrane segment (residues 668-688) close to its C-terminus, and is not secreted. Without being so limited, human PC7 amino acid and nucleotide sequences are shown in FIGS. 41A-B (SEQ ID NOs: 1-2) and 42A-D (SEQ ID NOs: 3-4); of rat PC7 amino acid and nucleotide sequences are shown in FIGS. 43 and 44 (SEQ ID NOs: 5-6); and of mouse PC7 amino acid and nucleotide sequences are shown in FIGS. 45 and 46A-G (SEQ ID NOs: 7-11).

As used herein the term “PC7 inhibitor” refers to an agent able to decrease PC7 expression (e.g., protein levels) and/or activity. Without being so limited, PC7 inhibitors include nucleic acid PC7 inhibitors. In some embodiments, the inhibition targets PC7 expression.

In specific embodiment, the above-mentioned PC7 inhibitors target hepatocytes to avoid side effects (target other tissues and/or decrease dose).

Nucleic Acid PC7 Inhibitors In specific embodiments, the nucleic acid PC7 inhibitor is a single-stranded antisense oligonucleotide (ASO), microRNA (miRNA) (non-coding RNA), or a dsRNA (e.g., RNAi, siRNA, miRNA (e.g., precursor or mimic, eventually processed into single stranded miRNA)) specific to PC7 mRNA, or a CRISPR-Cas base editor (cytosine, adenine or prime editor) or CRISPR-Cas prime editors, with guide RNAs (gRNAs) or prime editing guide RNAs (pegRNAs) targeting specific regions of the PCSK7 DNA locus.

While the present disclosure is not limited by any particular mechanism of action, in some embodiments, the nucleic acid enters a cell and causes the degradation, blocks the translation, blocks the interaction with another factor or affects the splicing of an RNA of complementary or identical sequences, including endogenous RNAs (mRNA or non-coding).

### Single-Stranded Antisense Oligonucleotides (ASOs)

In a specific embodiment, the PC7 inhibitor is an antisense oligonucleotide (ASO) specific for PC7 RNA (e.g., human). ASOs are synthetic single stranded strings of nucleic acids (natural or modified (e.g., Locked Nucleic Acid, phosphorothioate, 2′O-Methyl, 2′O-Methoxy, phosphoramidite, etc.)), between 8 and 50 nucleotides in length, preferably between 10 and 35, between 15 and 25, or 11, 12, 13, 14, 15, 16, 17, 18, 19, 20 in length. In some embodiments, the ASO is about 15, 16, 17, 18, 19, 20, 21, 22, 23, 24, 25 single-stranded nucleotides in length.

They bind to RNA through standard Watson-Crick base pairing. ASOs encompassed with the present invention interfere with PC7 RNA and result in its degradation (FIG. 47A-E) (e.g., gapmer); in preventing PC7 translation; in affecting splicing of the PC7 mRNA in a way that affects the resulting protein, and leading to a loss of function (e.g., ASO targeting PC7 splicing site); or in inhibiting the binding of a protein important for the processing of the mRNA (e.g., by targeting a binding partner's site on PC7). A non-exhaustive list of RNA-binding proteins (RBPs) that bind to PCSK7 mRNA according to the ENCODE database on RNA-binding protein (RBP) profiling based on enhanced crosslinking and immunoprecipitation assay include: AQR, BCLAF1. BUD13. DDX3X, EIF3H, IGF2BP1, LSM11, PABPN1, U2AF1, and SRSF1.

ASOs may target exons or introns. In specific embodiment ASO comprises a subsequence of a polynucleotide of the present disclosure (e.g., a subsequence of the sequence of FIGS. 42A-D (SEQ ID NOs: 2-4), FIG. 44 (SEQ ID NO: 6) or FIGS. 46A-G (SEQ ID NOs: 8-11). In other specific embodiments, the inhibitors are gapmers, i.e., ASOs that contains a central block of deoxynucleotide monomers sufficiently long to induce ribonuclease cleavage. In a specific embodiment, the ASOs of the present disclosure target exons. In specific embodiments, the ASOs of the present disclosure results in PC7 (e.g., >50% knock down of PC7) RNA degradation. Examples of ASOs specific for human PC7 and resulting in its degradation and targeting exon 3, 6, 8, 10, 12 or 14 are shown in FIG. 33B and ASOs specific for mice and targeting exons 2, 4 or 5 are shown in FIG. 35B. In more specific embodiments, the ASO targets exon 3, exon 12 or exon 14 of human PC7.

In specific embodiments, the above-mentioned PC7 ASOs are modified to target hepatocytes. For example, ASOs are conjugated at their 5′ and/or 3′ terminus with a GalNAc moiety. The GalNAc moiety includes but is not limited to aTriantennary GalNAc, Trivalent GalNAc (such as those produced by Trilink), Trishexylamino GalNac, etc. Examples of such GalNAc-conjugated ASOs are schematized in FIG. 48. In other specific embodiments, the GalNac are triantennary N-acetyl galactosamine as described in Prakash et al. Nucleic Acid Research, 2014 42(13) 8796-8807. In specific embodiments, GalNAc moieties can alternatively or in addition be attached to the membrane of lipidic vesicles (liposomes, etc.) encapsulating the ASOs.

In other embodiments, the above-mentioned PC7 inhibitors (e.g., PC7 ASOs, or miRNA mimics) are chemically modified to increase their stability and/or help them evade immune response (e.g., phosphorothioate (PS) (e.g., increases stability), 2′O-Methyl (2′OMe) (e.g., increases stability and reduces immune response), 2′O-Methoxy (2′MOE) e.g., (increases stability and reduces immune response), phosphoramidite (NP) (e.g., increases stability), locked nucleic acid or phosphoramidate morpholino (PMO), and peptide nucleic acid (PNA) groups. Such modifications may also assist in loading in the RISC complex and in excluding the passenger strand.

Double-Stranded RNA (dsRNA) Molecules

In a specific embodiment, the PC7 inhibitor is a double-stranded RNA (dsRNA) molecule (or a molecule comprising region of double-strandedness). In some embodiments, the dsRNA is about 15, 16, 17, 18, 19, 20, 21, 22, 23, 24, 25 or more duplex nucleotides in length. In some embodiments, the dsRNA is a small interfering RNA (siRNA), a short-hairpin RNA (shRNA), or a microRNA (miRNA) (miRNA mimics).

Without being so limited siRNA for use in methods disclosed herein include antisense molecules such as those prepared by Ionis pharmaceuticals and those available from Santa Cruz (e.g., sc-40889). Without being so limited, anti-PC7 RNAi inhibitors for use in methods disclosed herein include antisense molecules such as those prepared by Alnylam. Also provided herein are double-stranded RNA (dsRNA) molecules, comprising a portion of the mature polypeptide coding sequence of any one of the coding sequences of the polypeptides disclosed herein of inhibiting expression of that polypeptide in a cell. In a more specific embodiment, the inhibitor is a siRNA, e.g., of any one of SEQ ID NOs: 12-15 (FIG. 6A).

When a cell is exposed to a dsRNA, RNAs containing complementary sequences are selectively degraded by a process called RNA interference (RNAi). In some embodiments, dsRNAs provided herein are used in gene-silencing methods. In one aspect, methods are provided to selectively degrade RNA using the dsRNA is disclosed herein. In some embodiments, the PC7 inhibitor is a shRNA expressed by a DNA vector transfected or transduced into a target cell. In some embodiments, the PC7 inhibitor is a virus encoding a shRNA. In some embodiments, the PC7 inhibitor is a vector encoding a shRNA. The process is alternatively practiced in vitro, ex vivo or in vivo. In one aspect, the dsRNA molecules are used to generate a loss-of-function mutation in a cell, an organ or an organism. Methods for making and using dsRNA molecules to selectively degrade RNA are described in the art, see, for example, U.S. Pat. Nos. 6,506,559; 6,511,824; 6,515,109; and 6,489,127.

In some embodiments, a nucleic acid PC7 inhibitor (e.g., RNAi molecules, siRNA molecules, miRNA molecules, and analogues thereof) comprises a circular nucleic acid molecule, wherein the nucleic acid PC7 inhibitor (e.g., RNAi molecules, siRNA molecules, miRNA molecules, and analogues thereof) is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

In some embodiments, a circular a nucleic acid PC7 inhibitor (e.g., RNAi molecules, siRNA molecules, miRNA molecules, and analogues thereof) contains two loop motifs, wherein one or both loop portions of the dsRNA molecule (e.g., RNAi molecules, siRNA molecules, miRNA molecules, and analogues thereof) is biodegradable. In some embodiments, degradation of the loop portions of a circular a nucleic acid PC7 inhibitor (e.g., RNAi molecules, siRNA molecules, miRNA molecules, and analogues thereof) generates a double-stranded nucleic acid PC7 inhibitor (e.g., RNAi molecules, siRNA molecules, miRNA molecules, and analogues thereof) with 3-terminal overhangs, such as 3-terminal nucleotide overhangs comprising about 2 nucleotides. The sense strand of a double stranded dsRNA molecule (e.g., RNAi molecules, siRNA molecules, miRNA molecules, and analogues thereof) may have a terminal cap moiety such as an inverted deoxybasic moiety, at the 3-end, 5′-end, or both 3′ and 5′-ends of the sense strand.

In some embodiments, the 3-terminal nucleotide overhangs of an anti-PC7 dsRNA molecule (e.g., RNAi molecules, siRNA molecules, miRNA molecules, and analogues thereof) comprise ribonucleotides or deoxyribonucleotides that are chemically modified at a nucleic acid sugar, base, or backbone. In some embodiments, the 3-terminal nucleotide overhangs comprise one or more universal base ribonucleotides. In some embodiments, the 3-terminal nucleotide overhangs comprise one or more acyclic nucleotides.

In other specific embodiments, the PC7 inhibitor is an oligonucleotide with the nucleic acid sequence of a miRNA (regulatory) specific for PC7 RNA expression (e.g., human) (miRNA mimics). miRNAs are double-stranded RNAs (e.g., 18-22-nucleotide-long) that regulate gene expression post-transcriptionally by base-pairing with the 3′ untranslated region of target messenger RNAs and inhibiting their expression. The miRNA mimic technology (miR-Mimic) is innovative gene silencing approach to generate nonnatural double-stranded miRNA-like RNA fragments. Such an RNA fragment is designed to have its 5′-end bearing a partially complementary motif to the selected sequence in the 3′UTR unique to the target gene. Once introduced into cells, this RNA fragment, mimicking an endogenous miRNA, can bind specifically to its target gene and produce posttranscriptional repression, more specifically translational inhibition, of the gene. Unlike endogenous miRNAs, miR-Mimics act in a gene-specific fashion (Xiao, et al., 2007).

In the present disclosure, miRNAs (or miRNA mimics) are natural or modified (e.g., Locked Nucleic Acid, phosphorothioate, 2′O-Methyl, 2′O-Methoxy, phosphoramidite, etc.) double stranded strings of nucleic acids between 8 and 50 nucleotides in length, preferably between 10 and 35, between 15 and 25, between 18 and 22 or 11, 12, 13, 14, 15, 16, 17, 18, 19, 20 in length that target the PCSK7 transcript. In some embodiments, the miRNA is about 15, 16, 17, 18, 19, 20, 21, 22, 23, 24, 25 or more nucleotides in length targeting one or more regions of the PCSK7 mRNA. In specific embodiments, the oligonucleotide comprises or consists of the nucleic acids of miRNA (miRNA mimics) targeting one or more of (or two or more of or all three of) exon 14, exon 15 and 3′UTR of PCSK7. In some embodiments, the miRNA is about 15, 16, 17, 18, 19, 20, 21, 22, 23, 24, 25 or more single-stranded nucleotides in length. In a more specific embodiment, the oligonucleotide comprises or consist of the nucleic acids of miR-125a-5p, miR-143-3p, or miR-409-3p and any of their mimics (i.e., miRNA mimics of endogenous miRNAs). In a more specific embodiment, the oligonucleotide comprises or consists of the nucleic acids of miR-125a-5p.

### Base Editors

In a specific embodiment, the PC7 inhibitor is a CRISPR-Cas base editor (CRISPR cytosine base editor, adenine base editor, prime editor) used to induce loss- or gain-of-function variants of PC7 (e.g., comprising mutation in the prodomain, catalytic domain or the cytosolic tail such as but not limited to heterozygous or homozygous G65E, A102T, D187A, H228A, S505E, S505D or R504H, P777L variants).

CRISPR adenine base editors can induce targeted A→G edits in DNA (T→C on the opposing strand), whereas CRISPR cytosine base editors can induce targeted C→T edits in DNA (G→A on the opposing strand). These base editors use a nickase Cas protein fused to an evolved deoxyadenosine deaminase domain (adenine base editor) or a cytidine deaminase domain (cytosine base editor). Used with a guide RNA (gRNA) to engage a double-strand protospacer DNA sequence, flanked by a protospacer-adjacent motif (PAM) sequence on its 3′ end, these base editors will chemically modify an adenosine (adenine base editor) or cytosine (cytosine base editor) nucleoside on one DNA strand, which in combination with nicking on the other strand enables A→G or C→T transition mutations at the targeted site, respectively. Use of either base editor can inactivate genes by disrupting splice donors (a canonical GT sequence on the sense strand) or splice acceptors (a canonical AG sequence on the sense strand) at exon-intron boundaries, can change the binding site of a transcription factor in regulatory elements controlling the expression of the target gene or create loss or gain of function mutant version of the target gene.

Prime editing uses a catalytically impaired Cas nuclease (nickase) fused to an engineered reverse transcriptase in combination with a prime editing guide RNA (pegRNA) composed of a spacer sequence that hybridizes to the target DNA site and an extended template sequence for the reverse transcriptase which corresponds to the target site with the desired modification. Prime editors can be designed to induce all different base transversions as well as insertions or deletions of sequences, allowing to create loss of gain of function mutations in the PCSK7 gene.

For delivery to human hepatocytes, adenine base editor, cytosine base editor or prime editor mRNA together with relevant PCSK7 specific single-guide RNA or pegRNA are packaged into lipid nanoparticles (that may include a GalNAc moieties attached to the membrane of lipidic vesicles) and injected intravenously.

### Generation of Nucleic Acid PC7 Inhibitors

In some embodiments, a nucleic acid PC7 inhibitor molecule with sequences complementary to a target RNA is generated.

The method for synthesizing ASOs include solid-phase or enzymatic synthesis using phosphoramidite method and phosphoramidite building blocks derived from protected 2′-deoxynucleosides (dA, dC, dG, and T), ribonucleosides (A, C, G, and U), or chemically modified nucleosides, e.g., LNA or BNA. ASOs can be purified by e.g., gel electrophoresis or high-performance liquid chromatography (HPLC).

The methods of synthesizing synthesis dsRNA molecules comprise: (a) synthesis of two complementary strands of the dsRNA molecule; and (b) annealing the two complementary strands together under conditions suitable to obtain a double-stranded RNA molecule. In another embodiment, synthesis of the two complementary strands of the RNA molecule is by solid phase or enzymatic oligonucleotide synthesis. In yet another embodiment, synthesis of the two complementary strands of the RNA molecule is by solid phase tandem oligonucleotide synthesis. In some embodiments, a nucleic acid molecule described herein is synthesized separately and joined together post-synthetically, for example, by ligation or by hybridization following synthesis and/or deprotection. Oligonucleotides (e.g., certain modified oligonucleotides or portions of oligonucleotides lacking ribonucleotides) are synthesized using any suitable method. dsRNA constructs can be purified by gel electrophoresis or can be purified by HPLC. In some embodiments, an anti-PC7 dsRNA molecule (e.g., RNAi molecules, siRNA molecules, miRNA molecules, and analogues thereof) is assembled from two separate oligonucleotides, where one strand is the sense strand and the other is the anti-sense strand, wherein the anti-sense and sense strands are self-complementary (i.e. each strand comprises nucleotide sequence that is complementary to nucleotide sequence in the other strand; such as where the anti-sense strand and sense strand form a duplex or double stranded structure, for example wherein the double stranded region is about 19 base pairs). In some embodiments, the anti-sense strand of an anti-PC7 dsRNA molecule (e.g., siRNA molecules, miRNA molecules, and analogues thereof) comprises a nucleotide sequence that is complementary to a nucleotide sequence in a target nucleic acid molecule or a portion thereof, and the sense strand comprises a nucleotide sequence corresponding to the target nucleic acid sequence or a portion thereof. In some embodiments, an anti-PC7 dsRNA molecule (e.g., RNAi molecules, siRNA molecules, miRNA molecules, and analogues thereof) is assembled from a single oligonucleotide, where the self-complementary sense and anti-sense regions of the dsRNA molecule (e.g., RNAi molecules, siRNA molecules, miRNA molecules, and analogues thereof) are linked by means of a nucleic acid-based or non-nucleic acid-based linker(s). In some embodiments, an anti-PC7 dsRNA molecule (e.g., RNAi molecules, siRNA molecules, miRNA molecules, and analogues thereof) comprises a single stranded polynucleotide having nucleotide sequence complementary to nucleotide sequences in a target nucleic acid molecule or a portion thereof (for example, where such dsRNA molecule (e.g., RNAi molecules, siRNA molecules, miRNA molecules, and analogues thereof) does not require the presence within the dsRNA molecule (e.g., RNAi molecules, siRNA molecules, miRNA molecules, and analogues thereof) of nucleotide sequence corresponding to the target nucleic acid sequence or a portion thereof), wherein the single stranded polynucleotide further comprises a terminal phosphate group, such as a 5′-phosphate, or 5′,3′-diphosphate. The terminal structure of dsRNA molecules described herein is either blunt or cohesive (overhanging). In some embodiments, the cohesive (overhanging) end structure is a 3′ overhang or a 5′ overhang. In some embodiments, the number of overhanging nucleotides is any length as long as the overhang does not impair gene silencing activity. In some embodiments, an overhang sequence is not complementary (anti-sense) or identical (sense) to the PC7 sequence. In some embodiments, the overhang sequence contains low molecular weight structures (for example a natural RNA molecule such as tRNA, rRNA or tumor or CTC RNA, or an artificial RNA molecule).

The total length of dsRNA molecules having cohesive end structure is expressed as the sum of the length of the paired double-stranded portion and that of a pair comprising overhanging single-strands at both ends. For example, in the exemplary case of a 19 bp double-stranded RNA with 4 nucleotide overhangs at both ends, the total length is expressed as 23 bp. In some embodiments, the terminal structure of an anti-PC7 dsRNA molecule (e.g., RNAi molecules, siRNA molecules, miRNA molecules, and analogues thereof) has a stem-loop structure in which ends of one side of the double-stranded nucleic acid are connected by a linker nucleic acid, e.g., a linker RNA. In some embodiments, the length of the double-stranded region (stem-loop portion) is 15 to 49 bp, often 15 to 35 bp, and more commonly about 21 to 30 bp long.

In some embodiments, an anti-PC7 dsRNA molecule is a polynucleotide with a duplex, asymmetric duplex, hairpin or asymmetric hairpin secondary structure, having self-complementary sense and anti-sense regions, wherein the anti-sense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

### Selection of Nucleic Acid PC7 Inhibitors

In some embodiments, a nucleic acid PC7 inhibitor (e.g., ASOs, RNAi molecules, siRNA molecules, miRNA molecules, and analogues thereof) disclosed herein is capable of specifically binding to desired PC7 variants while being incapable of specifically binding to non-desired PC7 variants.

In some embodiments, a nucleic acid PC7 inhibitor (e.g., ASOs, RNAi molecules, siRNA molecules, miRNA molecules, and analogues thereof) is selected for use in a method disclosed herein based on predictions of the stability of molecule. In some embodiments, a prediction of stability is achieved by employing a theoretical melting curve wherein a higher theoretical melting curve indicates an increase in the molecule's stability and a concomitant decrease in cytotoxic effects. In some embodiments, stability of a nucleic acid PC7 inhibitor (e.g., ASOs, RNAi molecules, siRNA molecules, miRNA molecules, and analogues thereof) is determined empirically by measuring the hybridization of a single modified RNA strand containing one or more universal-binding nucleotide(s) to a complementary PC7 sequence within, for example, a polynucleotide array. In some embodiments, the melting temperature (i.e., the Tm value) for each modified RNA and complementary RNA immobilized on the array is determined and, from this Tm value, the relative stability of the modified RNA pairing with a complementary RNA molecule determined.

In some embodiments, a nucleic acid PC7 inhibitor (e.g., ASOs, RNAi molecules, siRNA molecules, miRNA molecules, and analogues thereof) is selected for use in a method disclosed herein based on “off-target” profiling whereby one or more nucleic acid molecules is administered to a cell(s), either in vivo or in vitro, and total RNA is collected, and used to probe a microarray comprising oligonucleotides having one or more nucleotide sequence from a panel of known genes, including non-target genes. The “off-target” profile of the modified RNA molecule (e.g., ASO, RNAi molecules, siRNA molecules, miRNA molecules, and analogues thereof) is quantified by determining the number of non-target genes having reduced expression levels in the presence of the nucleic acid molecule. The existence of “off target” binding indicates a nucleic acid PC7 inhibitor (e.g., ASOs, RNAi molecules, siRNA molecules, miRNA molecules, and analogues thereof) that is capable of specifically binding to one or more non-target gene. Ideally, a nucleic acid PC7 inhibitor (e.g., ASOs, RNAi molecules, siRNA molecules, miRNA molecules, and analogues thereof) applicable to therapeutic use will exhibit a high Tm value while exhibiting little or no “off-target” binding.

In some embodiments, a nucleic acid PC7 inhibitor (e.g., ASOs, RNAi molecules, siRNA molecules, miRNA molecules, and analogues thereof) is selected for use in a method disclosed herein by use of a report gene assay. In some embodiments, a reporter gene construct comprises a constitutive promoter, for example the cytomegalovirus (CMV) or phosphoglycerate kinase (PGK) promoter, operably fused to, and capable of modulating the expression of, one or more reporter gene such as, for example, a luciferase gene, a chloramphenicol (CAT) gene, and/or a β-galactosidase gene, which, in turn, is operably fused in-frame with an oligonucleotide (typically between about 15 base-pairs and about 40 base-pairs, more typically between about 19 base-pairs and about 30 base-pairs, most typically 20, 21, 22, 23, 24, 25, 26, 27, 28, or 29 base-pairs) that contains a target sequence for the one or more nucleic acid PC7 inhibitors. In some embodiments, individual reporter gene expression constructs are co-transfected with one or more nucleic acid PC7 inhibitors. In some embodiments, the capacity of a given a nucleic acid PC7 inhibitor (e.g., ASOs, RNAi molecules, siRNA molecules, miRNA molecules, and analogues thereof) to reduce the expression level of each of the contemplated gene variants is determined by comparing the measured reporter gene activity from cells transfected with and without the modified nucleic acid molecule.

In some embodiments, the nucleic acid inhibitor (e.g., ASOs, siRNA molecules, miRNA molecules, and analogues thereof) is selected for use in a method disclosed herein by assaying its ability to specifically bind to an RNA, such as an RNA (e.g., PCSK7 transcript) expressed by hepatocytes.

### Functional Characteristics of Inhibitors

As used herein the term “PC7 activity” refers to a direct or indirect PC7 activity that is independent from its enzymatic activity (proteolytic independent). More particularly, it refers to, without being so limited, PC7's increase of hepatic triglyceride levels; positive regulation of apoB levels in hepatocytes; positive regulation of apoB secretion from hepatocytes (Ex. 2, FIG. 6C, 7F-G); protection of apoB from ER-autophagy and/or lysosomal degradation (Ex. 2, FIGS. 6B-C, 7F); binding of PC7 with apoB (Ex. 2, FIG. 7H-1); and shedding specific membrane-bound substrate transferrin receptor 1 (hTfR1) (Ex. 8, FIGS. 23A-B), as well as the cancer associated protein CASC4 (PMID: 32820145).

The functional characteristics of the PC7 inhibitors (e.g., anti-PC7 dsRNA, ASOs, miRNAs) provided herein, in some embodiments, are tested in vitro and in vivo. For example, in some embodiments, PC7 inhibitors are tested for their ability to reduce lipid droplet size in hepatocytes or in the liver (see e.g., FIG. 2G, Ex. 2, FIG. 8; Ex. 1, FIGS. 2A-C; FIGS. 37A-B); reduce lipid droplet's volume (FIG. 39A, 2H-1, FIG. 8, 14L, FIG. 16D, FIG. 10G); reduce lipid droplet number (FIGS. 8, 14L, 16D, 39A); reduce plasma and hepatic TG levels (see e.g., FIGS. 4A, 2K, 10A, 18L); increase protein levels of Carnitine palmitoyl-transferase 1a (Cpt1a) (see e.g., Ex. 1, FIG. 4B); reduce ¹⁴C-oleic acid in hepatocytes vs. control (see e.g., Ex. 4, FIG. 12A); increase LC3-II levels (Ex. 4, FIG. 13); increase biogenesis of autophagosomes (Ex. 4, FIG. 13); increase ER-associated autophagy e.g., under normal diet (FIG. 12B); increase lipophagy in hepatocyte (FIG. 10E); increase hydrolysis of TG e.g., under normal diet (FIG. 11); enhance ß-oxidation in mitochondria (FIG. 4A); reduce circulating plasma apoB100 and apo48 levels and reduce in hepatic apoB100 and apo48 levels (Ex. 1, FIG. 2C-2D, Ex. 1, FIGS. 3A-B, Ex. 2, FIGS. 6B, 7A-B, FIG. 14B, FIGS. 14D-E, FIG. 38D); enhance co-translational degradation of apoB in hepatocytes (Ex. 1, FIG. 5A; Ex. 2, FIGS. 7C-7D); increase polyubiquitination of apoB100 (FIG. 7E); reduce apoB100 secretion (Ex. 1, FIG. 15C); increase generation of a secreted ˜50 kDa fragment of apoB in hepatocytes (Ex. 1, FIG. 5D); increase ER-60 protein level (FIG. 5F); increase ß-oxidation of fatty acids (FIG. 21, FIG. 10F); increased apoB degradation e.g., by the ERAD pathway (FIGS. 5E-F); decrease TG-rich very low-density lipoprotein (VLDL) particles (FIG. 6C; FIGS. 24A-B); regulate PC7 subcellular trafficking and localization to lysosomes (FIGS. 22A-C); reduce PC7 ability to shed specific membrane-bound substrate transferrin receptor 1 (hTfR1) (FIG. 2B; FIGS. 32A-B); reduce LDL-cholesterol (FIGS. 24A-B); increase body weight loss (FIG. 17G); reduce steatosis score (FIGS. 18B-D, 39B); reduce lobular/liver inflammation (FIGS. 18B-D, 39B); reduce hepatocyte ballooning (FIGS. 18B-D, 39B); reduce liver fibrosis score (FIGS. 18F, 18H, 39C); reduce liver toxicity through ALT (FIG. 18J); increase IRE1a mRNA level (FIGS. 10H, 19A); increase GRP78 mRNA level (FIG. 19A); increase IRE1a protein level FIG. 19D); decrease total liver Pcsk7 expression (e.g., mRNA) (FIGS. 38A-B), decrease liver/hepatocyte Pcsk7 expression (e.g., mRNA) (FIGS. 38B, 39D).

In some embodiments, the above assays are conducted with a mutant form of PC7 that further reduces Apoa5 intra- and/or extracellular concentration (s) (e.g., hPC7 S505E, S505D and R504H) or other mutants associated with reduced hepatic TGs (e.g., P777L). In some embodiments, PC7 inhibitors are tested for their impact on Apoa5 activity (e.g., reducing blood TG (circulating) and/or LDL levels).

In some embodiments, transgenic mice are genetically modified to express a hPC7. In some embodiments, PC7 inhibitors are tested in these models, or in animals which are not genetically modified, for the ability to reduce TG blood levels.

In some embodiments, the kinetics of TG clearance from plasma is determined by injecting animals with [¹²⁵I]-labelled TG, obtaining blood samples at 0, 5, 10, 15, and 30 minutes after injection, and quantitating [¹²⁵I]-TG in the samples.

Increased TG clearance in animals administered a PC7 inhibitors indicates that the agent inhibits PC7 Apoa5 activity in vivo.

In some embodiments, decreases in blood TGs in response to treatment with a PC7 inhibitor are indicative of therapeutic efficacy of the PC7 inhibitors. In some embodiments, lipid profiles are determined by colorimetric, gas-liquid chromatographic, or enzymatic means using commercially available kits.

As used herein, the term “decrease” or “reduction” (e.g., of a PC7 activity or of a NAFLD or NASH symptom) refers to a reduction of at least 10% as compared to a control, in an embodiment of at least 20% lower, in a further embodiment of at least 30% lower, in a further embodiment of at least 40% lower, in a further embodiment of at least 50% lower, in a further embodiment of at least 60% lower, in a further embodiment of at least 70% lower, in a further embodiment of at least 80% lower, in a further embodiment of at least 90% lower, in a further embodiment of 100% (complete inhibition).

Similarly, as used herein, the term “increase” or “increasing” (e.g., of a PC7 activity) of at least 10% as compared to a control, in an embodiment of at least 20% higher, in a further embodiment of at least 30% higher, in a further embodiment of at least 40% higher, in a further embodiment of at least 50% higher, in a further embodiment of at least 60% higher, in a further embodiment of at least 70% higher, in a further embodiment of at least 80% higher, in a further embodiment of at least 90% higher, in a further embodiment of 100% higher, in a further embodiment of 200% higher, etc.

The “control” for use as reference in the method disclosed herein is a cell (in the context of e.g., method of testing PC7 inhibitors in vitro or in cellulo) or subject (human or model animal) not treated with an inhibitor of the present disclosure). In the context of a method of preventing or treating a NAFLD (or NASH) or of a symptom thereof may be e.g., a control subject (or model animal) that has NAFLD (or NASH), and that is not treated with an agent present disclosure.

### Methods/Assays to Determine PC7 Activity are Described Below.

In some embodiments, PC7 inhibitors are tested for the ability to reduce PC7 activity on human hepatocyte cell lines HepG2 or HuH7. In some embodiments, the assay consists in the addition of wild type (WT), mutants or chimeric PC7, either transfected or purified, directly to the culture supernatants in the presence or absence of the tested compound. Each “dose-responses” experiment is done in triplicate for 4 to 6 different dosages.

WT PC7. In some embodiments, PC7 inhibitors are tested in an assay comprising the addition of wild type (WT) PC7, either as conditioned media from transfected cells or purified, and added to the culture supernatants, in the presence or absence of the PC7 inhibitor.

Mutants PC7 (gain of function). To further characterize whether the PC7 inhibitor inhibits the function of a gain of function mutation, the cells are incubated with purified mutant proteins, in the presence or absence of different doses of the PC7 inhibitor. In some embodiments, purified PC7 mutants are PC7 D374Y, S505E, S505D or R504H. In some embodiments, the doses chosen for PC7 and gain-of-function mutant such as D374Y, added extracellularly are 1 μg/ml and 0.2 μg/ml. In some embodiments, the assay is conducted using culture medium harvested from cells transfected with gain of function PC7 mutants.

### Formulations and Modes of Administration of PC7 Nucleic Acid Inhibitors

In some embodiments, compositions provided herein are administered by one or more routes of administration using one or more of a variety of suitable methods.

As will be appreciated by the skilled artisan, the route and/or mode of administration will vary depending upon the desired results. Routes of administration for PC7 inhibitors (e.g., anti-PC7 siRNA or ASOs) for uses and methods herein include, but are not limited to, intravenous, intraperitoneal, subcutaneous, intramuscular, intradermal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, PC7 inhibitors provided herein are administered by a nonparenteral route, such as a topical, epidermal or mucosal route of administration, for example, oral, intranasal, vaginal, rectal, sublingual or topical.

### ASOs Modes of Administration

When PC7 inhibitors of the present disclosure are ASOs, they can be administered parenterally or orally to subjects in need thereof in the form of naked nucleic acid or encapsulated nucleic acid (e.g., lipid-based particles such as liposomes) As indicated above, in specific embodiments, ASOs of the present invention are chemically modified to increase their nuclease resistance and avoid triggering an immune response and enable their administration as naked nucleic acid.

Parenteral routes appropriate for ASOs of the present disclosure include intravenous, intraperitoneal, or subcutaneous administrations.

### Double Stranded RNAs Modes of Administration

The present disclosure also encompasses vectors (plasmids) comprising the above-mentioned dRNAs (e.g., shRNA). The vectors are contemplated to be of any type suitable, e.g., for expression of said polypeptides or propagation of genes encoding said polypeptides in a particular organism. In some embodiments, the organism is of eukaryotic or prokaryotic origin. The specific choice of vector depends on the host organism and is known to a person skilled in the art. In an embodiment, the vector comprises transcriptional regulatory sequences or a promoter operably-linked to a nucleic acid comprising a sequence encoding PC7. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequence. Generally, operably-linked DNA sequences are contiguous and, where necessary to join two protein coding regions, in reading frame. However, since enhancers for example generally function when separated from the promoters by several kilobases and intronic sequences are often of variable lengths, some polynucleotide elements are operably-linked but not contiguous. “Transcriptional regulatory sequences” or “transcriptional regulatory elements” are generic terms that refer to DNA sequences, such as initiation and termination signals, enhancers, and promoters, splicing signals, polyadenylation signals, etc., which induce or control transcription of protein coding sequences with which they are operably-linked.

A recombinant expression vector comprising a double stranded nucleic acid sequence provided herein, in some embodiments is introduced into a cell, e.g., a host cell, which includes living cells capable of expressing a PC7 inhibitor provided herein encoded by a recombinant expression vector. Accordingly, also provided herein are cells, such as host cells, comprising the nucleic acid and/or vector as described above. The suitable host cell is any cell of eukaryotic or prokaryotic (bacterial) origin that is suitable, e.g., for expression of the nucleic acid. In some embodiments, the eukaryotic cell line is of mammalian, of yeast, or invertebrate origin. The specific choice of cell line is known to a person skilled in the art. Choice of bacterial strain will depend on the task at hand and is known to a person skilled in the art. The terms “host cell” and “recombinant host cell” are used interchangeably herein. Such terms refer not only to the particular subject cell, but also to the progeny or potential progeny of such a cell. Because certain modifications often occur in succeeding generations due to either mutation or environmental influences, such progeny are often not, in fact, be identical to the parent cell, but are still included within the scope of the term as used herein. Vectors are introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection, and viral-mediated transfection. Suitable methods for transforming or transfecting host cells is for example found in Sambrook et al. (supra), Sambrook and Russell (supra) and other laboratory manuals. Methods for introducing nucleic acids into mammalian cells in vivo are also known and are often used to deliver the vector DNA of a PC7 inhibitor provided herein to a subject for gene therapy.

The above-mentioned nucleic acid or vector, in some embodiments, is delivered to cells in vivo using methods well known in the art (such as direct injection of nucleic acid, receptor-mediated nucleic acid uptake, viral-mediated transfection or non-viral transfection and lipid based transfection), all of which often involve the use of gene therapy vectors. Direct injection has been used to introduce naked or chemically modified nucleic acid into cells in vivo. In some embodiments, a delivery apparatus (e.g., a “gene gun”) for injecting nucleic acid into cells in vivo is used. In some embodiments, such an apparatus is commercially available (e.g., from BioRad). In some embodiments, naked or chemically modified nucleic acid is introduced into cells by complexing the nucleic acid to a cation, such as polylysine, which is coupled to a ligand for a cell-surface receptor. Binding of the nucleic acid-ligand complex to the receptor, in some embodiments, facilitates uptake of the nucleic by receptor-mediated endocytosis. A nucleic acid-ligand complex linked to adenovirus capsids which disrupt endosomes, thereby releasing material into the cytoplasm, in some embodiments, is used to avoid degradation of the complex by intracellular lysosomes.

Defective retroviruses are well characterized for use as gene therapy vectors (for a review see Miller, A. D., Blood 76:271 (1990)). Protocols for producing recombinant retroviruses and for infecting cells in vitro or in vivo with such viruses are found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include psiCrip, psiCre, psi2 and psiAm. Retroviruses have been used to introduce a variety of genes into many different cell types, including epithelial cells, endothelial cells, lymphocytes, myoblasts, hepatocytes, bone marrow cells, in vitro and/or in vivo.

For use as a gene therapy vector, the genome of an adenovirus, in some embodiments, is manipulated so that it encodes and expresses a nucleic acid of a PC7 inhibitor provided herein (e.g., a nucleic acid encoding an anti-PC7 a dsRNA targeting PC7) but is inactivated in terms of its ability to replicate in a normal lytic viral life cycle. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and are often used to infect a wide variety of cell types, including airway epithelium, endothelial cells, hepatocytes, and muscle cells.

In some embodiments, adeno-associated virus (AAV) is used as a gene therapy vector for delivery of DNA for gene therapy purposes. AAV is a naturally occurring defective virus that requires another virus, such as an adenovirus or a herpes virus, as a helper virus for efficient replication and a productive life cycle. In some embodiments, AAV is used to integrate DNA into non-dividing cells. In some embodiments, lentiviral gene therapy vectors are adapted for use in methods provided herein.

DsRNAs of the present disclosure (e.g., siRNA) can also be administered to subjects in need thereof as nucleic acid encapsulated in lipid-based particles such as liposomes.

### Pharmaceutical Compositions

In certain embodiments, the PC7 inhibitors (e.g., anti-PC7 siRNAs or ASOs) provided herein are formulated to ensure proper distribution in vivo.

For example, the therapeutic compounds provided herein, in some embodiments, are formulated in lipid-based nanoparticles (e.g., liposomes). In some embodiments, the liposomes comprise one or more moieties which are selectively transported into specific cells (e.g., hepatocytes), tissues or organs (e.g., liver), thus enhance targeted drug delivery (see, e.g., Ranade V. V., J. Clin. Pharmacol. 29:685 (1989)). In an embodiment, the PC7 inhibitors provided herein are formulated to be delivered to the liver (i.e., to hepatocytes). Without being so limited, nucleic acid can be encapsulated in lipid-based nanoparticles that can optionally contain GalNac on their surface to target liver. Biodegradable, biocompatible polymers used in some embodiments, such as lipids, ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. In some embodiments, therapeutic compositions are administered with medical devices known in the art.

In another aspect, compositions are provided, e.g., a pharmaceutical composition, comprising one or a combination of PC7 inhibitors (e.g., anti-PC7 siRNA or ASO) provided herein, formulated together with a pharmaceutically acceptable carrier and/or excipient.

As used herein, “pharmaceutically acceptable carrier” or “pharmaceutically acceptable excipient” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the PC7 inhibitor coated in a material to protect the compound from the action of acids and other natural conditions that, in some embodiments, inactivate the compound.

The pharmaceutical compositions provided herein, in some embodiments include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci. 66:1-19 (1977)). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Pharmaceutical compositions provided herein, in some embodiments, include a pharmaceutically acceptable antioxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and non-aqueous carriers that are employed in the pharmaceutical compositions of provided herein include, but are not limited to, water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity is maintained, for example, by the use of coating materials, such as lecithin, by the maintenance of the required particle size in the case of dispersions, and by the use of surfactants.

In some embodiments, compositions herein contain adjuvants such as preservatives, wetting agents, emulsifying agents, and dispersing agents. Prevention of presence of microorganisms is ensured, in some embodiments, both by sterilization procedures, supra, and by the inclusion of various antibacterial and antifungal agents, for example, paraben, chlorobutanol, phenol sorbic acid, and the like. In some embodiments, it is desirable to include isotonic agents, such as sugars, sodium chloride, and the like into the compositions. In addition, prolonged absorption of the injectable pharmaceutical form, in some embodiments, is brought about by the inclusion of agents which delay absorption such as, aluminum monostearate and gelatin.

Pharmaceutically acceptable carriers or excipients include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions provided herein is contemplated. In some embodiments, supplementary active compounds are incorporated into the compositions.

Therapeutic compositions typically must be sterile and stable under the conditions of manufacture and storage. In some embodiments, the composition is formulated as a solution, microemulsion, liposome, or other ordered structure suitable to high drug concentration. In some embodiments, the carrier is a solvent or dispersion medium containing, for example, water, ethanol, polyol (e.g., glycerol, propylene glycol, and liquid polyethylene glycol, and the like), and suitable mixtures thereof. The proper fluidity is maintained, for example, by the use of a coating such as lecithin, by the maintenance of the required particle size in the case of dispersion and by the use of surfactants. In many cases, isotonic agents are included, for example, sugars, polyalcohols such as mannitol, sorbitol, or sodium chloride in the composition.

Prolonged absorption of the injectable compositions is brought about by including in the composition an agent that delays absorption for example, monostearate salts and gelatin.

Sterile injectable solutions are prepared, in some embodiments, by incorporating the PC7 inhibitor (e.g., anti-PC7 siRNA or ASO) in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of PC7 inhibitor (e.g., anti-PC7 siRNA or ASO) to be combined with a carrier material to produce a single dosage form will vary depending upon the subject being treated, and the particular mode of administration. The amount of active ingredient combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

### Dosages and Regimen

Dosage regimens are adjusted to provide the optimum desired response (e.g., a therapeutic response). For example, in some embodiments, a single bolus is administered. In some embodiments, several divided doses are administered over time. In some embodiments, the dose is proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of PC7 inhibitors provided herein are dictated by and directly dependent on the unique characteristics of the active compound and the particular therapeutic effect to be achieved, and the limitations inherent in the art of compounding such an active compound for the treatment of sensitivity in individuals.

In some embodiments, for administration of a PC7 inhibitor (e.g., anti-PC7 siRNA or ASO), exemplary dosage ranges include but are not limited to from about 0.0001 to about 100 mg/kg of the host body weight. In some embodiments, dosage ranges include from about 0.01 to about 5 mg/kg of the host body weight. In some embodiments, dosages are about 0.3 mg/kg body weight, about 1 mg/kg body weight, about 3 mg/kg body weight, about 5 mg/kg body weight, or about 10 mg/kg body weight or within the range of about 1-10 mg/kg. An exemplary treatment regime entails administration once per week, once every two weeks, once every three weeks, once every four weeks, once a month, once every 3 months or once every three to 6 months. Exemplary dosage regimens PC7 inhibitors (e.g., anti-PC7 siRNA or ASO) provided herein include, but are not limited to about 1 mg/kg body weight or about 3 mg/kg body weight by intravenous administration.

The PC7 inhibitor (e.g., anti-PC7 siRNA or ASO) is usually administered on multiple occasions. Intervals between single dosages are, for example, weekly, monthly, every three months or yearly. In some embodiments, intervals are irregular as indicated by measuring blood levels of PC7 inhibitor (e.g., anti-PC7 siRNA or ASO), in the patient. In some methods, dosage is adjusted to achieve a plasma concentration of the PC7 inhibitor (e.g., anti-PC7 siRNA or ASO), of about 1-1000 μg/ml and in some methods about 25-300 μg/ml.

Alternatively, PC7 inhibitors (e.g., anti-PC7 siRNA or ASO) are administered as a sustained release formulation, in which case less frequent administration is required. Dosage and frequency vary depending on the half-life of the inhibitor in the patient. The dosage and frequency of administration varies, in some embodiments, depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, in some embodiments, the patient is administered a prophylactic regime.

Actual dosage levels of the active ingredients in the pharmaceutical compositions provided herein, in some embodiments, are varied so as to obtain an amount of the active ingredient which is effective to achieve the desired therapeutic response (e.g., decreased hepatic TG levels) for a particular individual, composition, and mode of administration, without being toxic to the individual. The selected dosage level will depend upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

### Combinations

Pharmaceutical compositions provided herein, in some embodiments, are administered in combination therapies, i.e., combined with other agents or therapies known to prevent or treat NAFLD or symptom(s) thereof.

In specific embodiments PC7 inhibitors (e.g., anti-PC7 siRNA or ASOs) are administered together with another therapy known to prevent or treat NAFLD or at least a symptom thereof or another TG-related disorder (e.g., therapeutic agent or non-pharmacological therapies such as weight loss, done through a combination of calorie reduction, exercise, and dietary modifications).

In specific embodiments PC7 inhibitors (e.g., anti-PC7 siRNA or ASOs) are administered together with another therapeutic agent known to prevent or treat NAFLD or at least a symptom thereof or another TG-related disorder. Such compositions, in some embodiments, include one or a combination of (e.g., two or more different) PC7 inhibitors. In some embodiments, pharmaceutical compositions provided herein comprise one or a combination of ASOs. In some embodiments, the combination therapy comprises a PC7 inhibitor (e.g., an ASO o ranti-PC7 siRNA) combined with at least one triglyceride-reducing agent.

Other agents potentially useful for preventing or treating NAFLD or at least a symptom thereof include omega 3 fatty acids, anti-cholesterol agents, etc. Examples of active ingredients including agents useful for preventing or treating a TG-related disorder that, in some embodiments, are administered in combination with a PC7 inhibitor provided herein include, but are not limited to, other compounds which improve a patient's lipid profile, such as (a) HMG-CoA reductase inhibitors, (e.g., statins, including lovastatin, simvastatin, fluvastatin, rosuvastatin, pravastatin, rivastatin, atorvastatin, itavastatin, pitavastatin, cerivastatin and other statins); (b) cholesterol absorption inhibitors, such as stanol esters, beta-sitosterol, sterol glycosides such as tiqueside; and azetidinones, such as ezetimibe; (c) inhibitors of cholesterol ester transport protein (CETP) (e.g., anacetrapib or dalcetrapib) which are now in clinical trials to increase HDL and decrease LDL cholesterol; (d) niacin and related compounds, such as nicotinyl alcohol, nicotinamide, and nicotinic acid or a salt thereof; (e) bile acid sequestrants (cholestyramine, colestipol (e.g., colestipol hydrochloride), dialkylaminoalkyl derivatives of a cross-linked dextran, Colestid®, LoCholest®; (f) acyl CoA:cholesterol acyltransferase (ACAT) inhibitors, such as avasimibe and melinamide, and including selective ACAT-1 and ACAT-2 inhibitors and dual inhibitors; (g) PPARy agonists, such as gemfibrozil and fenofibric acid derivatives (fibrates), including clofibrate, fenofibrate, bezafibrate, ciprofibrate, and etofibrate; (h) microsomal triglyceride transfer protein (MTP)/ApoB secretion inhibitors, (i) anti-oxidant vitamins, such as vitamins C and E and beta carotene; (k) thyromimetics; (l) LDL receptor inducers; (m) platelet aggregation inhibitors, for example glycoprotein IIb/IIIa fibrinogen receptor antagonists and aspirin; (n) vitamin B 12 (also known as cyanocobalamin), (o) folic acid or a pharmaceutically acceptable salt or ester thereof, such as the sodium salt and the methylglucamine salt, (p) FXR and LXR ligands, including both inhibitors and agonists, (q) agents that enhance ABCA1 gene expression, (r) ileal bile acid transporters; and (s) PCSK9 inhibitors such as anti-PCSK9 monoclonal antibodies (evolocumab and alirocumab), siRNA (inclisiran), vaccine, ASO or small molecules targeting PCSK9.

In some embodiments, the two are alternatively administered sequentially in either order; or administered simultaneously (in the same composition or in different compositions).

In some embodiments, the combination therapy regimen is additive. In some embodiments, the combination therapy regimen produces synergistic results (e.g., reductions in hepatic TG levels greater than expected for the combined use of the two agents). In some embodiments, combination therapy with a PC7 inhibitor (e.g., anti-PC7 siRNA or ASO) and another agent that prevents or treats NAFLD or a symptom thereof produces synergistic results (e.g., synergistic reductions in hepatic TG levels). In some subjects, this allows reduction in the dosage of the other agent for preventing or treating NAFLD and/or an agent useful for preventing or treating a TG-related disorder (“other active agent”) to achieve the desired TG levels. PC7 inhibitors, in some embodiments, are useful for subjects who are intolerant to therapy with the other active agent, or for whom therapy with the other active agent has produced inadequate results (e.g., subjects who experience insufficient TG reduction on statin therapy).

### Diseases

In specific embodiments, the subject has a NAFLD or a symptom thereof. NAFLD is a reversible condition wherein large vacuoles of triglyceride fat accumulate in liver cells via the process of steatosis. NASH is a type of NAFLD condition of fatty liver characterized by ballooning, inflammation, and fibrosis. (See FIG. 1 showing the progression from healthy liver to fatty liver and NASH liver). As used herein the term “NAFLD symptom” encompasses, without being so limited, one or more of steatosis, liver/lobular inflammation, hepatocyte ballooning, fibrosis, pancreatitis and diabetes. In specific embodiments, the disease is diet induced NAFLD. In a specific embodiment, the NAFLD symptom encompasses, one or more of fibrosis, steatosis, liver/lobular inflammation, and hepatocyte ballooning.

As used herein the term “TG-related disorder” refers to disorders in which HTG is a factor or a symptom including, without being so limited cardiovascular disorders (CVDs), type 2 diabetes mellitus (T2D), fibrosis, metabolic syndrome, and acute pancreatitis. Reducing of TG and increasing HDL-c is an important component of primary and secondary programs for preventing CVDs, NAFLD, atherogenic dyslipidemia or T2D.

### Subjects

As used herein the term “subject” is meant to refer to any animal, such as a mammal including human, mice, rat, dog, cat, pig, cow, monkey, horse, etc. In a particular embodiment, it refers to a human.

As used herein the terms “subject is a likely candidate for NAFLD” and “subject is a likely candidate for NASH” is meant to refer to obese subjects, subjects having alcoholism and subjects with genetic predisposition for NAFLD or NASH or a triglyceride related disorder, subjects with a family history comprising NAFLD or NASH or a triglyceride related disorder.

### Treatment and Prevention

The terms “treat/treating/treatment” and “prevent/preventing/prevention” as used herein, refers to eliciting the desired biological response, i.e., a therapeutic and prophylactic effect, respectively. In accordance with the disclosure herein, the therapeutic effect comprises one or more of a decrease/reduction in the severity of a human disease (e.g., a reduction hepatic triglycerides (TGs) levels), a decrease/reduction in at least one NAFLD symptom (e.g., reduced number/amount of lipid droplets in liver, etc.), an amelioration of at least one NAFLD symptom, following administration of the at least one PC7 inhibitor (e.g., anti-PC7 siRNA or ASO) encompassed herein, or of a composition comprising the PC7 inhibitor, in combination with another agent for the prevention or treatment of a NAFLD or a symptom thereof. In accordance with the disclosure provided herein, in some embodiments, a prophylactic effect comprises a complete or partial avoidance/inhibition of excess hepatic TGs or of at least one NAFLD symptom following administration of the at least one PC7 inhibitor encompassed herein, or of a composition comprising the PC7 inhibitor, in combination with another agent for the prevention or treatment of a NAFLD or a symptom thereof.

In some embodiments, “therapeutically effective amount” or “effective amount” or “therapeutically effective dosage” of PC7 inhibitor (e.g., anti-PC7 siRNA or ASO) provided herein results in a lowering of hepatic TG level in a subject, a decrease in severity of at least one NAFLD symptom, an increase in frequency and duration of disease symptom-free periods, or a prevention of impairment or disability due to the disease affliction in the subject.

### Method of Detection

In an embodiment, anti-PC7 antibodies are used to detect the presence or levels of PC7. In some embodiments, this is achieved by contacting a sample (e.g., a biological sample such as blood, serum, plasma, or a cell sample) with the anti-PC7 antibody under conditions that allow for the formation of a complex between the anti-PC7 antibody and PC7. Any complexes formed between the antibody and PC7 are detected and compared in the sample and in a control sample. For example, standard detection methods, well known in the art, such as ELISA and flow cytometric assays, are contemplated to be performed using the anti-PC7 antibodies disclosed herein.

Accordingly, in one aspect, there are provided methods for detecting the presence of PC7 (e.g., hPC7) in a sample, or measuring the amount of PC7 (e.g., active form of PC7), comprising contacting the sample with an anti-PC7 antibody provided herein, under conditions that allow for formation of a complex between the anti-PC7 antibody and PC7. The formation of a complex is then detected, wherein a difference in complex formation between the sample compared to a control sample is indicative of the presence of PC7 in the sample.

### Kits

Also provided herein are kits comprising (A) a PC7 inhibitor (e.g., anti-PC7 siRNA or ASOs) provided herein or the compositions provided herein, and (B) (i) another agent for the prevention or the treatment of a non-alcoholic fatty liver disease (NAFLD) or a symptom thereof; (ii) a pharmaceutically acceptable carrier and/or excipient; (iii) instructions for use or a combination thereof; or (iv) a combination of at least two of (i) to (iii). In some embodiments the kit further comprises a least one additional reagent, or one or more additional PC7 inhibitor(s) provided herein. Kits typically include a label indicating the intended use of the contents of the kit. The term label includes any writing, or recorded material supplied on or with the kit, or which otherwise accompanies the kit. In some embodiments, the kit further comprises one or more container(s), reagent(s), administration device(s) (e.g., a syringe).

The disclosure herein is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present disclosure and claims. The contents of all references, including issued patents and published patent applications, cited throughout this application are hereby incorporated by reference in their entirety.

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1: Schematic representation of the progression from healthy liver to fatty liver, to NASH liver, cirrhotic liver, and hepatocellular carcinoma.

FIGS. 2A-J: (FIGS. 2A-B) Total plasma total cholesterol (FIG. 2A) and triglycerides (TG) (FIG. 2B) levels in wild type (WT) and Pcsk7−/− (KO) mice on chow diet. (FIGS. 2C-D) apoB100 protein levels in plasma (FIG. 2C) and liver (FIG. 2D) and apoB liver mRNA levels (FIG. 2D) in wild type (WT) and Pcsk7−/− (KO) mice fed chow diet. (FIG. 2E) On high fat diet (HFD) Western blots of apoB100 and MTP protein levels in WT and Pcsk7−/− mice on high fat diet (HFD). Levels are normalized to tubulin. (FIG. 2F) Liver content of lipid droplets (LD) and triglycerides of WT and Pcsk7−/− mice on HFD. (FIGS. 2G-K) Total LD (LD, TG-rich vesicles) accumulation (FIG. 2G), LD content (total area stained) (FIG. 2H), mean size (mean area (FIG. 2I), total number of LD (mean count) (FIG. 2J), total liver TG (FIG. 2K) in wild type (WT) and Pcsk7−/− (KO) mice on chow diet. FIGS. 3A-B: (FIG. 3A) Under normal diet (ND), Pcsk7 KO mice exhibit ˜50% lower circulating apoB100 and apoB48 levels, without any change in hepatic microsomal triglyceride transfer protein (MTP) levels (not shown) or apoB mRNA levels. ApoB protein expression detected by western blotting showing the apoB100 (500 kDa) and apoB48 (250 kDa). FIG. 3B: ApoB mRNA levels shown graphically.

FIGS. 4A-B: (FIG. 4A) Hepatic lipids were extracted from mice livers, and TG concentrations were measured (8-11 mice per group). (FIG. 4B) Western blot quantification of hepatic proteins for: total acetyl-coenzyme A carboxylase 1, Acc1 (tAcc1), the ratio of the inhibitory phosphorylated Acc1 (pAcc1c) to total Acc (tAcc1), as well as for Carnitine palmitoyltransferase 1a (Cpt1a), a rate-limiting enzyme for b-oxidation of FA. Values are means±SEM. Statistical comparisons with controls were performed by an unpaired two-sided t-test. *p<0.05. ***p<0.001.

FIGS. 5A-F: Pcsk7 KO mice exhibit enhanced degradation in primary hepatocytes. (FIG. 5A-D) Pulse-chase analysis of hepatic apoB from media and cell lysate of WT and Pcsk7^{-/-} primary hepatocytes (n=2 independent experiments performed in duplicate). (FIGS. 5A-B) Co-translational degradation of apoB in Pcsk7−/− hepatocytes. (FIGS. 5C-D) ApoB100 secretion associated with the increased generation of a secreted ˜50 kDa fragment of apoB in Pcsk7−/− hepatocytes over chase time. (FIGS. 5E-F) Western blot analysis of ER-60 protein levels in livers extracts of WT and Pcsk7 KO mice, shown gel (FIG. 5E) and graphically (FIG. 5F). Statistical comparisons with controls were performed by an unpaired two-sided t-test. *p<0.05, ***p<0.001.

FIGS. 6A-C: (FIG. 6A) Oligonucleotide sequences of a PCSK7-siRNA pool (siRNA sequences 1, 2, 3 and 4 (SEQ ID NOs: 12-15). (FIG. 6B) Knockdown of PC7 via siRNA reduces secreted ApoB levels from immortalized human hepatocyte (IHH) & HepG2 cells, based on WB analysis. (FIG. 6C) ApoB associated TG VLDL secretion (IHH cells). IHH cells were pulse-labeled for 2 h with ¹⁴C-oleate and secreted ¹⁴C-labeled VLDL particles were quantified. Statistical comparisons with controls were performed by an unpaired two-sided t-test. **p<0.05.

FIGS. 7A-l: PCSK7 can bind apoB in the ER and acts as a molecular chaperone-like facilitating its secretion, and its absence leads to a rapid degradation of apoB by the proteasome. IHH cells that do not express PCSK7 were generated by CRISPR knockout (PCSK7^{-/-}). (FIG. 7A) Western blot (presented on gel and graphically) of apoB100 and PC7 protein levels in WT and PCSK7^{-/-} IHH cells. Levels are normalized to α-tubulin. (FIG. 7B) Western blot (presented on gel and graphically) of apoB100 and PC7 protein levels over time (0, 15 and 30 minutes) in WT and PCSK7^{-/-} IHH cells incubated with cycloheximide (CHX) to block de novo mRNA translation. Levels are normalized to α-tubulin. (FIG. 7C-D) Western blot (presented on gel and graphically) of cellular apoB levels in WT and PCSK7^{-/-} IHH cells incubated with brefeldin A (BFA) that blocks exit from the endoplasmic reticulum (ER), an autophagy inhibitor 3-methyladenine (3MA), proteasome inhibitors MG132, Lactacystin, DMSO or non treated (NT) controls. (FIG. 7E) Western blot (presented on gel) of cellular apoB levels and polyubiquitinated apoB100 in WT and PCSK7^{-/-} IHH cells incubated with Lactacystin. (FIG. 7F) Schematic representation of apoB100 and apoB21 protein. Western blots (presented on gel and graphically) of apoB100 and PC7 protein levels in WT and PCSK7^{-/-} naïve IHH cells overexpressing PCSK7 and a secretory short N-terminal fragment of apoB (apoB21; aa 1-965 aa) with a cell permeable pan-proprotein convertase inhibitor decanoyl-RVKR-cmk, that blocks PCSK7 enzyme activity. Levels are normalized to β actin. (FIG. 7G) Western blot (presented on gel) of apoB21 (s-apoB) protein levels in naïve WT IHH cells co-expressing rat PCSK7 (rPC7) and its soluble-KDEL variant (retained in the ER) (rPC7-KDEL). Levels are normalized to α-tubulin. (7H) Western blot (presented on gel) of cellular apoB levels in IHH cells overexpressing PC7 (tagged with V5 at the C-terminus). Cell lysates were immunoprecipitated with a mAb-V5 followed by a Western blot (WB) with an apoB100 antibody. (FIG. 7I). Western blot (presented on gel) of endogenous apoB with V5-tagged human PC7 in IHH cells overexpressing PC7-V5 versus those that either express 7B2-V5 or SKI-1-V5 as controls. Cell lysates were immunoprecipitated with a human ApoB antibody and the complex was separated by SDS-PAGE and co-IP proteins revealed by Western blot to the V5 tag. PC7 binds directly apoB since it can be pulled down with endogenous ApoB.

FIG. 8: CRISPR silencing of PCSK7 in IHH cells reduces the level & size of intracellular lipid droplets and enhances cell proliferation. Control IHH cells and those silenced for endogenous PC7 by CRISPR were incubated with oleic acid for 72 h and the cells were then washed and analyzed for TG levels by oil-red-0 labeling.

FIGS. 9A-W: qPCR analysis of the mRNA levels of 13 representative genes in hepatocytes mice WT (white bars) and Pcsk7 KO mice (black bars) fed a CHOW or NASH diet for 12 or 16 weeks.

FIGS. 10A-H: (FIG. 10A) Plasma TG levels measured in vivo following administration of the detergent poloxamer-407 (P-407), which blocks TG-lipolysis. At 6 h after injection of P-407 the plasma TG levels were ˜50% lower in Pcsk7^{-/-} mice, revealing a significantly reduced hepatic secretion of VLDL-TG in Pcsk7^{-/-} mice. (FIG. 10B) Primary hepatocytes of WT and Pcsk7^{-/-} mice incubated with ³H-acetate for 2 h, the ³H-TG extracted, and the de novo incorporated radioactivity counted. No significant difference in de novo lipogenesis between WT and Pcsk7^{-/-} could be detected. (FIGS. 10C-D) Pyruvate kinase (PKLR), liver receptors for TG-enriched VLDL (LDL receptor-related protein 1, LRP1), scavenger receptor for plasma non-esterified free FA (NEFA) and lipoproteins (CD36) are unchanged in WT and Pcsk7^{-/-} mice. (FIG. 10E) Significant increase in functional macroautophagy-lysosome pathway in primary hepatocytes of Pcsk7^{-/-} mice, whereby levels of LC3-II are higher both in absence and presence of chloroquine, suggesting enhanced functional lipophagy. (FIG. 10F) FA oxidation potential compared in human hepatic IHH cells expressing or completely lacking PCSK7 (IHH-CRISPR PCSK7 KO). The cells were incubated with ¹⁴C-oleate and the released ¹⁴CO₂ was captured at different time points, thereby revealing enhanced FA β-oxidation (increased ¹⁴CO₂) both after 8 h and 12 h from the start of the incubation. (FIG. 10G) IHH and IHH-CRISPR PCSK7 KO cells incubated overnight with 0.3 mM oleic acid (OA), washed and incubated in medium lacking OA for 88 h. Cells then stained with Oil-red 0 revealing the loss of LD-staining in absence of PCSK7, as observed Pcsk7^{-/-} mice. (FIG. 10H) Comparison of protein levels of ER-stress markers in IHH versus IHH-CRISPR PCSK7 KO cells. The data showed higher levels of phospho-IRE1α and phospho-PERK in cells lacking PCSK7, suggesting an activated unfolded protein response (UPR) signaling in absence of PCSK7. In summary, the upregulated UPR signaling connects the observation of lower apoB to the loss of the potential apoB chaperone PCSK7, higher autophagy, and to lower lipid accumulation in hepatocytes via enhanced β-oxidation.

FIG. 11: Schematic representation of PC7 absence: enhanced autophagy, decreased apoB levels and steatosis.

FIGS. 12A-B: Primary hepatocytes from Pcsk7 KO mice accumulate less fat, do not show a difference in lipogenesis, but do exhibit a 6-7 folds enhanced autophagy and enhanced fatty acid β-oxidation. (FIG. 12A) Pcsk7 KO hepatocytes seem to be resistant to accumulating Fat. Primary hepatocytes of KO and WT mice were incubated with radiolabeled oleic acid [¹⁴C], TG content was analyzed at different time points after lipid extraction. (FIG. 12B). No change in lipogenesis in absence of PC7. Primary hepatocytes were incubated with ¹³C-acetate for 24 h and total cellular radiolabeled lipids were quantified.

FIG. 13: Immunoblots of LC3 in primary hepatocytes of WT and Pcsk7^{-/-} mice incubated for 6 h without or with 100 M chloroquine (n=2 independent experiments performed in triplicates). Values are means±SEM. Statistical comparisons with controls were performed by an unpaired two-sided t-test. *p<0.05, **p<0.01, ***p<0.001.

FIGS. 14A-I. Effect of the loss of PCSK7 expression on the extent fat accumulation in the liver, following a Western diet high in fat, fructose, and cholesterol (HFFC), which induces a robust NAFLD-like phenotype in mice. (FIG. 14A) Schematic representation of experiment performed. Four 8-week-old groups of 20 WT and 20 Pcsk7−/− mice were fed a chow or HFFC diet for 12-weeks. At the end of the 12-week feeding period, half of them (20-weeks old) were sacrificed for assessment of the development of NAFLD, and the other half were allowed to recover under a chow diet for another 4 weeks (until 24-weeks old). (FIGS. 41B-E) apoB100 levels were reduced in Pcsk7−/− mice compared to WT at the end of the 16-week periods (12+4 weeks) under chow diet, or post-recovery for 4 weeks following an H FFC diet for 12-weeks in plasma (FIGS. 41B-C) and liver (FIGS. 41D-E). The data show that in both plasma and liver, apoB100 levels are ˜50-60% lower in Pcsk7−/− mice compared to WT, with no significant change in apoB48. (FIGS. 41F-G) Oil red 0 (ORO) staining of liver sections revealed a robust accumulation of LD following an H FFC diet in both genotypes, suggesting the lack of PCSK7 does not prevent the occurrence of diet induced NAFLD. (FIGS. 41H-1) In contrast, following the recovery period the absence of PCSK7 dramatically reduced the extent of LD accumulation, evidenced by the reduced total area stained by ORO, decreased mean area, and increased number of LD.

FIGS. 15A-B: Western blot analyses of apoB levels in (FIG. 15A) plasma and (FIG. 15B) liver of mice fed a chow or western diet high in fat, fructose, and cholesterol (HFFC) diet (NASH) for 12 weeks.

FIGS. 16A-F: (FIGS. 16A-B) 12-week-old mice fed a chow diet exhibited no significant change in LD in Pcsk7^{-/-} mice versus WT. (FIGS. 16C-D) In contrast, 16-week-old mice fed a chow diet exhibited a significant ˜20% reduction in LD in Pcsk7^{-/-} mice versus WT. (FIGS. 16E-F) 12-week-old mice fed a HFFC diet (FIG. 16E) or 12-week-old mice fed a HFFC followed by a 4-weeks recovery under a chow diet (FIG. 16F) did not any difference in total liver cholesterol between both genotypes.

FIGS. 17A-M: (FIG. 17A) comparison of weight gain between PCSK7+/+(WT), PCSK7−/− (KO) mice after (i) 20 weeks of normal diet (CHOW Diet) and (ii) after 8 weeks of normal diet followed by 12 weeks of high fat/sugar(fructose)/cholesterol (HFFC) diet (NASH diet) (WT: n=10; KO: n=10); (FIG. 17B) comparison of weight gain between PCSK7+/+(WT), PCSK7−/− (KO) mice after (i) 24 weeks of normal diet (CHOW Diet) and (ii) after 8 weeks of normal diet followed by 12 weeks of high fat/sugar(fructose)/cholesterol (HFFC) diet (NASH diet) followed by recovery period of 4 weeks of normal diet (WT: n=8; KO: n=6); (FIG. 17C) comparison of liver weight/body weight between PCSK7+/+(WT), PCSK7−/− (KO) mice after (i) 20 weeks of normal diet (CHOW Diet) and (ii) after 8 weeks of normal diet followed by 12 weeks of high fat/sugar/cholesterol diet (NASH diet) (WT: n=10; KO: n=10); (FIG. 17D) comparison of liver weight/body weight between PCSK7+/+(WT), PCSK7−/− (KO) mice after (i) 24 weeks of normal diet (CHOW Diet) and (ii) after 8 weeks of normal diet followed by 12 weeks of high fat/sugar(fructose)/cholesterol (HFFC) diet followed by recovery period of 4 weeks of normal diet (NASH diet) (WT: n=8; KO: n=6); (FIG. 17E) comparison of perigonadal weight between PCSK7+/+(WT), PCSK7−/− (KO) mice after (i) 20 weeks of normal diet (CHOW Diet) and (ii) after 8 weeks of normal diet followed by 12 weeks of high fat/sugar(fructose)/cholesterol (HFFC) (NASH diet) (WT: n=10; KO: n=10); (FIG. 17F) comparison of perigonadal weight between PCSK7+/+(WT), PCSK7−/− (KO) mice after (i) 24 weeks of normal diet (CHOW Diet) and (ii) after 8 weeks of normal diet followed by 12 weeks of high fat/sugar(fructose)/cholesterol (HFFC) diet followed by recovery period of 4 weeks of normal diet (NASH diet) (WT: n=8; KO: n=6); (FIG. 17G) percentage of body weight loss after (i) 24 weeks of normal diet (CHOW Diet) and (ii) after 8 weeks of normal diet followed by 12 weeks of high fat/sugar(fructose)/cholesterol (HFFC) (NASH diet) followed by a recovery of 4 weeks of normal diet (WT: n=8; KO: n=6) excluding non-responders; (FIG. 17H) representative images of 18-30 images of Oil Red 0 staining in PCSK7+/+(WT) and PCSK7−/− (KO) mice after (i) 20 weeks of normal diet (CHOW Diet) and (ii) after 8 weeks of normal diet followed by 12 weeks of high fat/sugar(fructose)/cholesterol (HFFC) diet (NASH diet), scale bar: 20 μm; (FIG. 17I) graphical comparison of quantification of LDs by Oil Red O staining (FIG. 17I) and number of lipid droplets (LD) (FIG. 17J) between PCSK7+/+(WT), PCSK7−/− (KO) mice after (i) 20 weeks of normal diet (CHOW Diet) and (ii) after 8 weeks of normal diet followed by 12 weeks of high fat/sugar(fructose)/cholesterol (HFFC) diet (NASH diet) (WT: n=10; KO: n=10); (FIG. 17K) representative images of 18-30 images of quantification of lipid droplets (LDs) staining in PCSK7+/+(WT) and PCSK7−/− (KO) mice after (i) 24 weeks of normal diet (CHOW Diet) and (ii) after 8 weeks of normal diet followed by 12 weeks of high fat/sugar(fructose)/cholesterol (HFFC) diet followed by four weeks of normal diet (NASH diet), scale bar: 20 μm; (FIG. 17L-M) graphical comparison of quantification of LDs between PCSK7+/+(WT), PCSK7−/− (KO) mice after (i) 24 weeks of normal diet (CHOW Diet) and (ii) after 8 weeks of normal diet followed by 12 weeks of high fat/sugar(fructose)/cholesterol (HFFC) diet (NASH diet) followed by four weeks of normal diet (WT: n=10; KO: n=10), *p<0.05, **p<0.01, ***p<0.0001, ****p<0.00001, *****p<0.000001.

FIG. 18AM: (FIGS. 18A-B) Liver tissue image (FIG. 18A) and steatosis score, lobular inflammation, and hepatocyte ballooning (hallmarks of NASH-associated pathology) (FIG. 18B) from WT and Pcsk7^{-/-} mice liver after 12 weeks of an HFFC diet. (FIGS. 18C-D) Liver tissue image (FIG. 18C) and steatosis score, lobular inflammation, and hepatocyte ballooning (FIG. 18D) from WT and Pcsk7^{-/-} mice liver after 12 weeks of an HFFC diet followed by a 4-week recovery period on the chow diet. (FIGS. 18E-F) Liver tissue image (FIG. 18E) and fibrosis score (FIG. 18F) from WT and Pcsk7^{-/-} mice liver after 12 weeks of an HFFC diet. (FIGS. 18G-H) Liver tissue image (FIG. 18G) and fibrosis score (FIG. 18H) from WT and Pcsk7^{-/-} mice liver after 12 weeks of an HFFC diet followed by a 4-week recovery period on the chow diet. (FIGS. 181-J) Liver toxicity as assessed by the levels of circulating ALT (alanine aminotransferase) from WT and Pcsk7^{-/-} mice liver after 12 weeks of a chow diet or HFFC diet (FIG. 18I) or after 16 weeks of a chow diet or a 12 weeks of an HFFC diet followed by a 4-week recovery period on the chow diet (FIG. 18J). (FIGS. 18K-L) Liver TG content in WT and Pcsk7^{-/-} mice after 12 weeks of a chow diet or HFFC diet (FIG. 18K) or after 16 weeks of a chow diet or a 12 weeks of an HFFC diet followed by a 4-week recovery period on the chow diet (FIG. 18L). (FIG. 18M) Log 2-transformed averaged signal intensity was performed (https://software.broadinstitute.org/morpheus). The lipid changes observed under the HFFC diet in a WT background are not observed in Pcsk7−/− mice. Altogether, the latter findings suggest that the loss of Pcsk7 expression reversed the hepatic lipid perturbations induced by the HFFC diet.

FIGS. 19A-F: (FIGS. 19A-C) mRNA level of ER-stress marker IRE1a (FIG. 19A), ER-chaperone GRP78 (FIG. 19A), PERK (FIG. 19B), sXBP1 (FIG. 19B) or ATF6 (FIG. 19C), measured at the 12-week and 16-week periods in WT and Pcsk7^{-/-} (KO) mice on chow diet (for 12 or 16 weeks) or HFFC diet (for 12 weeks or for 12 weeks followed by 4-week recovery period on chow diet) (NASH). (FIG. 19D) Protein level of ER-stress marker IRE1a, measured at the 12-week and 16-week periods in WT and Pcsk7^{-/-} (KO) mice on HFFC diet (for 12 weeks or for 12 weeks followed by 4-week recovery period on chow diet) (NASH). (FIG. 19E) Protein level of ER-stress marker IRE1a, measured at the 12-week and 16-week periods in WT and Pcsk7^{-/-} (KO) mice on chow diet (for 12 or 16 weeks). (FIG. 19F) Schematized proposed model wherein PCSK7 acts as an apoB100 chaperone to allow its efficient exit from the ER and wherein its absence results in an increased UPR activation leading to enhanced autophagy and lipid β-oxidation, leading to reduced lipid accumulation in the liver (lower levels of LD).

FIGS. 20A-B: Western blot analyses of MTP and TM6SF2 levels in livers of WT and Pcsk7^{-/-} mice (FIG. 20A) after 12 weeks of chow or HFFC (NASH) diet and (FIG. 20B) after 12 weeks of chow or of HFFC (NASH) diet followed by 4 weeks recovery period. Level of control α-tubulin also shown.

FIGS. 21A-B: (FIG. 21A) Associations between gain of function PCSK7 SNP (rs236918) and cardiometabolic traits. (FIG. 21B) Associations between loss of function PCSK7 SNP (rs201598301) resulting in Proline-777-Leucine and lipid characteristics in humans, including P777L heterozygous individuals B007 and B041. V. Sachan & R. Essalmani: collaboration with May Faraj (IRCM).

FIGS. 22A-D: (FIG. 22A) Schematic representation of human PC7 P777L variant. (FIG. 22B) Western blot analyses of apoB and PC7 in IHH cells overexpressing either a control empty vector, a cDNA expressing human PC7 in absence or presence (+RVKR-cmk) of its protease inhibitor. The relative levels of apoB100 to that of the control empty vector are shown after normalization of tubulin levels; (FIG. 22C) IHH cells were co-transfected with cDNAs encoding a short apoB construct tagged with V5 at the C-terminus (s-apoB; 965 aa) together with either an empty vector or one expressing PC7 or its variant P777L in absence or presence of 10 mM NH4Cl. Western blot analyses of cells and media for apoB (V5) and PC7 are shown. Normalized levels to actin are presented as bar graphs below. (FIG. 22D) Table showing the plasma characteristics of 5 female (F) and 3 male (M) subjects exhibiting low levels of TG, apoB, apoB/PCSK9 and transferrin receptor 1 (TfR1).

FIGS. 23A-B: (FIG. 23A) Schematic representation of the primary structure of the active form (92 kDa) of human PC7 showing the catalytic, the transmembrane domain (M), and the C-terminal cytosolic tail (C) with the positions of the variation P777L, the 4 N-glycosylation sites and the two Cys-palmitoylation sites. (FIG. 23B) HEK293 cells were co-transfected with cDNAs coding for hTfR1 and either WT PC7 or its cytosolic tail (CT) P777L variant. The next day cell lysates and media were analyzed for the shedding of full length hTfR1 into a soluble, secreted sTfR1. The P777L form results in 50% LOF on the shedding of human transferrin Receptor 1 (hTfR1) into a soluble form shTfR1, compared to WT. Other CT variants are shown for comparison. These include the soluble form of PC7 (no shedding activity), its form that lacks the CT (delta-CT, 80% LOF), the one that lacks partially (C699A+C704A; 20% LOF) or completely (C673A+C699A+C704A; 50% LOF) Cys-palmitoylation (Durand L. et al. JBC 2019; PMID:31915245).

FIGS. 24A-B: Fast Protein Liquid Chromatography (FPLC) profiles. For each male, 300 mL of plasma from either a normal (RE) or a B041 patient carrying the heterozygote PC7-P777L mutation were separated by FPLC (Pharmacia, Sweden) on a Superose™ 6 HR10/30 column (Pharmacia Biotech) with a flow rate of 0.3 ml/min. In each fraction (FIG. 24A) Total Cholesterol (TC) and (FIG. 24B) Triglyceride (TG) profiles were deduced from measurements of total cholesterol (TC) and triglycerides (TG) with commercial kit (Sobioda). Elution position of VLDL, LDL and HDL shown on the chromatogram. Optical densities were obtained on a SpectraMax™ i3 plate reader (Molecular Devices).

FIGS. 25A-B: Schematic view of the genome organization of PCSK7. (FIG. 25A) Exons, introns, and miRNA target sites on the PCSK7 sequence. The presence of two putative sites targeted by miR-125-5p on exons 14 and 15 is shown by a horizontal bar below the schematized E14 and E15 exons. Depicts the target sequences of the four miRNAs (SEQ ID NOs: 16-21) (hsa-miR-125-5p (SEQ ID NO: 22), hsa-miR-143-3p (SEQ ID NO: 23), hsa-miR-409-3p (SEQ ID NO: 24), and hsa-miR-320a-3p (SEQ ID NO: 25) in the 3′-UTR of PCSK7 and their nucleotide hybridization status. miR-125-5p and miR-143-3p each target two sites in the 3′-UTR (see PMID: 35888711). (FIG. 25B) Expression levels of PCSK7 mRNA and predicted microRNAs in three examined cell lines, HEK293T, HepG2, and Huh7, assessed by qPCR analysis. The expression level of PCSK7 mRNA is the highest in Huh7, whereas miR-143-3p and miR-409-3p have the lowest expression in all examined cell lines (see PMID: 35888711).

FIGS. 26A-B: Expression level of PCSK7 after miRNA overexpression in Huh7 (FIG. 27A) and HEK293T (FIG. 27B) cell lines compared with their mock vector counterpart. The expression level of PCSK7 mRNA in Huh7 cells was significantly downregulated by miR-143-3p and miR-409-3p. In both Huh7 and HEK293T cell lines, miR-125a-5p downregulated PCSK7 expression, although the difference did not reach statistical significance in Huh7 cells (P=0.06). In addition, miR-320a-3p could not downregulate the expression of PCSK7 in either Huh7 or HEK293T cells. *p<0.05, and **p<0.01 (see PMID: 35888711).

FIG. 27: Luciferase assay results following co-transfection of the miR-overexpressing vector and the 3′-UTR of the wild-type PCSK7 in HEK293T cells. Relative luciferase activity considerably decreased after the overexpression of mir-125a-5p, miR-143-3p, and miR-409-3p; however, there was no significant alteration in relative luciferase activity for miR-320a-3p and miR-224-5p, which were used as controls. *p<0.05, **p<0.01, ***p<0.001 (see PMID: 35888711).

FIGS. 28A-C: Luciferase assay analysis of cells co-transfected with the miR-overexpressing vector and miRNA target site-deleted (Mut) psiCHECK-2 vectors in the HEK293T cells. The results demonstrated a direct interaction of miR-125a-5p, miRNA-143-3p, and miR-409-3p with the 3′-UTR of PCSK7. (FIG. 28A) Both target sites of miR-125a-5p in the 3′-UTR were functional. There was no significant alteration in relative luciferase activity in cells transfected with miR-125a-5p, Mut-a,b in the 3′-UTR of the psiCHECK-2 vector, in which both target sites in miR-125a-5p were omitted. Relative luciferase activity significantly decreased following miR-125a-5p over-expression in Mut-a,b in the 3′-UTR of the psiCHECK-2 vector, in which only one target site of miR-125a-5p was deleted. (FIG. 28B) The two miRNA-143-3p target sites in the 3′-UTR of PCSK7 are functional. No significant alteration was found in relative luciferase activity in cells transfected with the miR-143-3p-overexpressing vector and Mut-a,b in the 3′-UTR of the psiCHECK-2 vector, in which both miR-143-3p target sites were deleted. However, relative luciferase activity significantly diminished following the co-transfection of the miR-143-3p-overexpressing vector and Mut-a,b in the 3′-UTR of the psiCHECK vector, in which only one target site was absent. (FIG. 28C) No significant changes were observed in luciferase activity after the deletion of the only target site of miR-409-3p in the 3′-UTR of PCSK7. Mut-a stands for the deletion of the first target site, Mut-b stands for the deletion of the second target site, and Mut-a,b stands for the deletion of both miRNA target sites. *p<0.05, and **p<0.01 (see PMID: 35888711).

FIGS. 29A-B: Western blot (FIG. 29A) and quantitative (FIG. 29B) analysis of human PC7 expression in Huh7 cells, in which miR-125a-5p and miR-224-5p were overexpressed. The expression of PC7 protein was significantly diminished by miR-125a-5p at 48 hours post-transfection compared to mock counterpart vectors (PEGFP-C1), whereas miR-224-5p did not change the protein levels of PC7. **p<0.01 (see PMID: 35888711).

FIGS. 30A-B: Western blot (FIG. 30A) and quantitative (FIG. 30B) analysis of the protein expression of PCSK7 following the co-transfection of the PC7-overexpressing vector, along with different ratios of the miR-125a-5p-overexpressing vectors (1:2 [2×], 1:3 [3×], and 1:5 [5×]) in HuH7 cells. The expression of PC7 protein decreased significantly (***p<0.001) in all three ratios of the miR-125a-5p-overexpressing plasmid, whereas there was no significant difference concerning the reductions of PCSK7 in the different amounts of miR-125a-5p (see PMID: 35888711).

FIGS. 31A-B. Western blot (FIG. 31A) and quantitative (FIG. 31B) analysis of the cellular expression of human TfR1 after the co-transfection of the miR-125a-5p and TfR1-overexpressing vectors in Huh7 cells. No direct effect by miR-125a-5p on TfR1 expression was observed. Here miR-125a-5p was overexpressed at different plasmid DNA ratios of PCSK7: miRNA: 1:2 (2×), 1:3 (3×), and 1:5 (5×). X stands for vector DNA fold for miR-125-5p compared to vector expressing PC7.

FIGS. 32A-B Western blot (FIG. 32A) and quantitative (FIG. 32B) analysis of human PC7 and TFR1 expression in Huh7 cells, in which miR-125-5p was overexpressed. FIG. 32A shows that the levels of shed soluble sTfR1-V5 in the media significantly decreased (***p<0.001) due to the reduction of PC7 protein levels detected by Western blot in the cells. miR-125a-5p was overexpressed at different plasmid DNA ratios of PCSK7:miRNA: 1:2 (2×), 1:3 (3×), and 1:5 (5×). X stands for vector DNA fold for miR-125-5p compared to vector expressing PC7. Because both PC7-V5 and hTfR1-V5 were V5-tagged, the WB using a mAb-V5 reflects the expression of both proteins. However, WB using a PC7-specific antibody clearly showed that miR-125-5p reduced cellular PC7 protein levels, without any effect on hTfR1 levels (see PMID: 35888711).

FIGS. 33A-D: Design of antisense oligonucleotides targeting human PCSK7 mRNA. (FIG. 33A) Schematic representation of the human PCSK7 gene with the location where the 8 antisense oligonucleotides (ASO) designed and synthesized to target the RNA. (FIG. 33B) Sequences for each of the tested human targeted PCSK7 ASOs 1 to 8 (SEQ ID NOs: 27-34) (in this order from top to bottom) are indicated together with the position of chemical modifications at each nucleotide: 56-FAM, fluorophore; m, 2′O-Methyl (protection against nuclease degradation and immune response); *, phosphorothioate (PS) (enhanced stability & increased binding to serum proteins preserves oligonucleotides in circulation, slows removal by the liver and boosts the time available for uptake into target tissues). (FIG. 33C) Illustrative structure of phosphorothioate and phosphodiester linkages. (FIG. 33D) Endogenous expression levels of PCSK7 RNA in 4 cells lines, normalized to IHH, as assessed by RT-qPCR.

FIGS. 34A-E: RNA and protein knockdown efficiency of ASOs targeting human PCSK7. Human PCSK7 mRNA levels (FIGS. 34A-B) in human hepatocyte IHH cells, assessed by RT-qPCR and protein levels (FIG. 34C) were assessed by Western blot (using a PC7 antibody), 48 h after transfection of 25 nM ASO (FAM labeled) or siRNA pool (siRNA sequences 1, 2, 3 and 4 in FIG. 6A). Tubulin was used as loading control. For RT-qPCR, RNA levels were assessed in non-sorted bulk cells (FIG. 34A) as well as in FAM-positive sorted cells (FIG. 34B). Expression values were normalized to transfection reagent only conditions. FIGS. 34D-E: Transfection of 25 nM ASOs in in human hepatocyte IHH cells. Protein knockdown efficiency of ASOs targeting human PCSK7, assessed by Western blot 72 h (FIG. 34D) and 96 h (FIG. 34E) post transfection, respectively. Tubulin was used as loading control.

FIGS. 35A-B: Design of Antisense Oligos targeting mouse Pcsk7 mRNA. (FIG. 35A) Schematic representation of the mouse Pcsk7 gene with the location where the 8 antisense oligos (ASO) designed and synthesized target the RNA. (FIG. 35B). Sequences for each of the tested mouse PCSK7 targeted ASOs 1 to 8 (SEQ ID NOs: 35-42) (in this order) are indicated together with the position of chemical modifications at each nucleotide: 56-FAM, fluorophore; m, 2′O-Methyl (protection against nuclease degradation and immune response); *, phosphorothioate (PS) (enhanced stability & increased binding to serum proteins preserves oligonucleotide in circulation, slows removal by the liver and boosts the time available for uptake into target tissues).

FIGS. 36A-B: RNA knockdown efficiency of ASOs targeting mouse Pcsk7. Mouse Pcsk7 mRNA levels (FIG. 36A) and viability (FIG. 36B) in mouse FL83B hepatocyte cells, assessed by real time-qPCR 48 h after transfection with Dharmafect™ of 25 nM ASO. RNA levels were assessed in non-sorted bulk cells as well as in FAM-positive sorted cells (not shown). Expression values were normalized to transfection reagent only conditions. *p<0.05, **p<0.01, ***p<0.001, and ****p<0.0001. The cell viability of the FL83B cells is presented in FIG. 27B.

FIGS. 37A-E: Comparison of various ASOs and vitamin E on lipid droplet accumulation in mouse FL38B hepatocytes. (FIG. 37A) Immunocytochemical analysis of mouse FL38B hepatocyte cells incubated for 48 h with oleic acid in the presence of either fluorescent mouse mASO-1, mASO-2 or mASO-7 (not shown) or α-tocopherol (vitamin E) (arrows pointing towards transfected cells, red staining indicate lipid droplets). (FIG. 37B) Quantitative analysis of results expressed in bar graph. (FIGS. 37C-D). The effect of eight ASOs on Pcsk7 mRNA levels as measured by quantitative PCR (qPCR). The experiment was repeated two independent times. *p<0.05 (FIG. 37A) The effect of mASO-1, mASO-2, mASO-7 and Vitamin E on reduction of accumulation of fluorescent triglycerides in cells as estimated by FACS measurement of lipid droplets (LD) by LipidTox™ staining. (FIG. 37E). modifications of ASO by trivalent linkage to N-acetyl-Galactosamine (GlcNac).

FIGS. 38A-D: Effect of 8 different ASOs (see FIG. 35B) in mouse FL38B hepatocytes. (FIG. 38A) Dose-dependent efficacy of these ASOs for their ability to decrease total liver Pcsk7 mRNA following their subcutaneous injection in mice at 2, 5, 10 and 15 mg/kg, twice a week for 1 month. (FIG. 38B) Livers and their partially purified primary hepatocytes of mice injected with 5 mg/kg of either GalNac-ASO-7 and GalNac-ASO-2, twice a week for 1 month, analyzed by RT-qPCR for levels of Pcsk7 mRNA. (FIG. 38C) Schematic representation of experiment performed whereby 8 weeks-old mice were fed an HFFC diet for 16 weeks (until 24 weeks-old), after which they were switched to a regular chow diet for another 8-week recovery period (until 32 weeks-old). At the 24 weeks-old period, preceding the recovery period, the mice were divided into 3 groups of 12 mice each: (1) a control non-treated group; (2) two GalNac-ASO groups subcutaneously injected twice a week for 8 weeks with either GalNac-ASO-7 or GalNac-ASO-2 at 5 mg/kg; and (3) a group fed a chow diet containing Vitamin E (α-Tocopherol) at 500 IU/kg of food. (FIG. 38D) Analysis of the levels of apoB in the plasma of the above 24- (before recovery) and 32-weeks-old mice (after recovery) revealed that after recovery both GalNac-ASO2 and GalNac-ASO7 reduced plasma apoB100 by >60%, very similar to the levels observed in Pcsk7−/− mice, supporting the notion that it is hepatocyte-derived PCSK7 that primarily regulates circulating apoB levels. In each of FIGS. 38A-B and D, the top to bottom order of the legend corresponds to the left to right order in the histogram of each tested condition (e.g., in FIG. 38A, results obtained with ASO NT in total liver are illustrated in the first column, results obtained with ASO 2 are illustrated in the second column, and results obtained with ASO 7 are illustrated in the third column),

FIGS. 39A-D (FIG. 39A) Upper panels—Oil red 0 staining of liver sections control (no treatment), and ASO-2, ASO-7 and α-tocopherol treated mice. Lower panels; Total area of LDs, mean area of LD and LD number in non treated mice or in mice treated with GalNac-ASO-7, GalNac-ASO-2 or α-tocopherol. (FIG. 39B) Liver morphology and fat accumulation measured in terms of liver inflammation, hepatocyte ballooning and steatosis grade in non treated mice or in mice treated with GalNac-ASO-7, GalNac-ASO-2 or α-tocopherol. (FIG. 39C) Extent of fibrosis measured by Sirius red staining in non treated mice or in mice treated with GalNac-ASO-7, GalNac-ASO-2 or α-tocopherol. (FIG. 39D) Levels of Pcsk7 mRNAs in livers after the 8-week recovery period that followed the 16-week HFFC diet in non treated mice or in mice that had been treated with GalNac-ASO-7, GalNac-ASO-2 or α-tocopherol. In each of FIGS. 39A and D, the top to bottom order of the legend corresponds to the left to right order in the histogram of each tested condition (e.g., in FIG. 39A, results obtained with ASO NT in total Area Stained are illustrated in the first column, results obtained with ASO-2 are illustrated in the second column, results obtained with ASO-7 are illustrated in the third column and results obtained with alpha-tocopherol are illustrated in the fourth column).

FIGS. 40A-B: Determination of liver or toxicity in non treated mice or in mice treated with GalNac-ASO-7, GalNac-ASO-2 or α-tocopherol measured by levels either (FIG. 40A) AST (Aspartate Aminotransferase) or (FIG. 40B) ALT (Alanine Aminotransferase). In each of FIGS. 40A and B, the top to bottom order of the legend corresponds to the left to right order in the histogram (e.g., in FIG. 40A, results obtained with the Non treated animals are illustrated in the first column, results obtained with ASO 2 are illustrated in the second column, results obtained with ASO 7 are illustrated in the third column and results obtained with tocopherol are illustrated in the fourth column).

FIGS. 41A-B: An amino acid sequence of human PC7 (SEQ ID NO: 1) (FIG. 41A). The signal peptide is highlighted in grey, the propeptide is bolded; the extracellular domain is underlined; the transmembrane domain is italicized; and the cytoplasmic domain is in lower case letters. In FIG. 41B, the amino acid sequence of FIG. 41A is reproduced, further indicating known natural polymorphisms (highlighted) and mutants (bolded) disclosed herein below the relevant lines of the sequences.

FIGS. 42A-D: (FIG. 42A) A nucleotide sequence of human PC7 disclosed in FIG. 41A (SEQ ID NO: 2). In FIG. 42A, the nucleotide sequence encoding the protein is shown underlined (SEQ ID NO: 26) of which the nucleotide sequence encoding the signal peptide is highlighted in grey. The fragments corresponding to the siRNA of FIG. 6A are in bold (wherein the siRNA uracil are depicted as thymines). (FIGS. 42B-C) Other human PC7 isoform(SEQ ID NO: 3). (FIG. 42D) Other human PC7 isoform (SEQ ID NO: 4).

FIG. 43: An amino acid sequence of rat PC7 (SEQ ID NO: 5). The signal peptide is highlighted in grey, the propeptide is bolded; the extracellular domain is underlined; the transmembrane domain is italicized; and the cytoplasmic domain is in lower case letters.

FIG. 44: A nucleotide sequence of rat PC7 disclosed in FIG. 43 (SEQ ID NO: 6). The nucleotide sequence encoding the protein sequence is shown underlined of which the nucleotide sequence encoding the signal peptide is highlighted in grey.

FIG. 45: An amino acid sequence of mouse PC7 (SEQ ID NO: 7). The signal peptide is highlighted in grey, the propeptide is bolded; the extracellular domain is underlined; the transmembrane domain is italicized; and the cytoplasmic domain is in lower case letters.

FIGS. 46A-G: (FIG. 46A) A nucleotide sequence of mouse PC7 disclosed in FIG. 44 (SEQ ID NO: 8). The nucleotide sequence encoding the protein sequence is shown underlined of which the nucleotide sequence encoding signal peptide is highlighted in grey. (FIGS. 46B-C) Other mouse PC7 isoform (SEQ ID NO: 9). (FIGS. 46D-E) Other mouse PC7 isoform (SEQ ID NO: 10). (FIGS. 46F-G) Other mouse PC7 isoform (SEQ ID NO: 11).

FIGS. 47A-E: Schematization of ASOs' mechanism of action (prior art—https://www.nature.com/articles/nrneurol.2017.148).

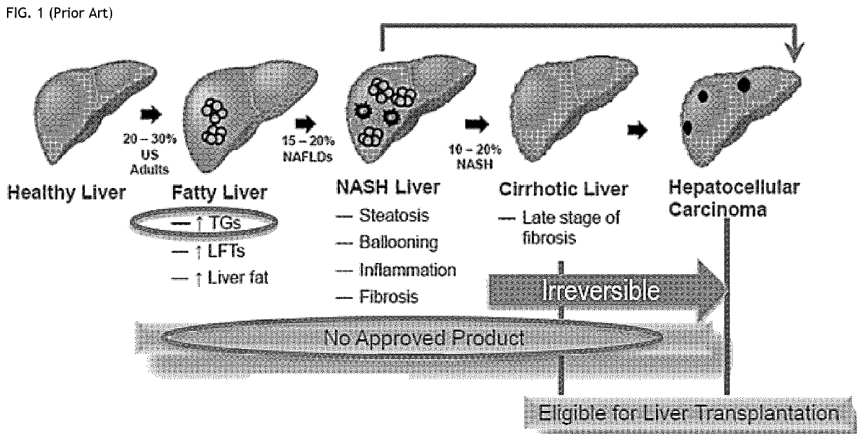

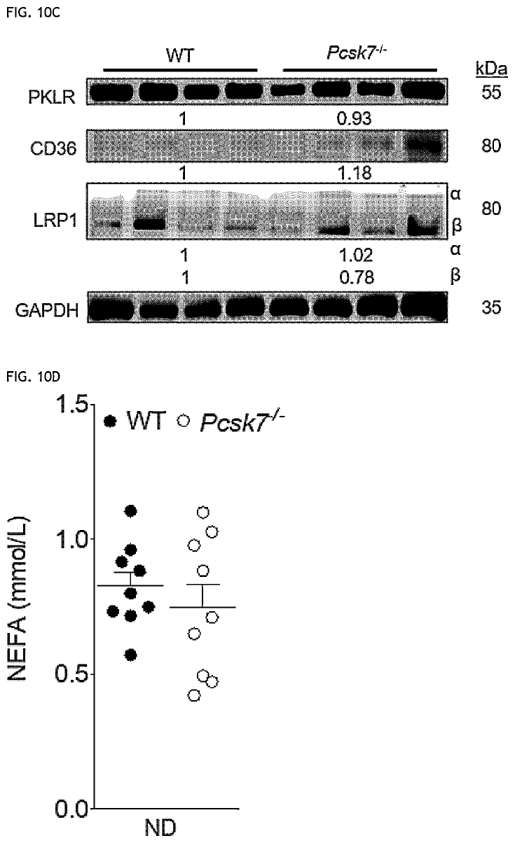

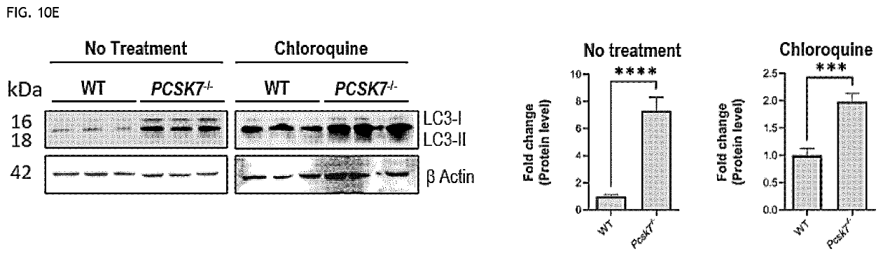

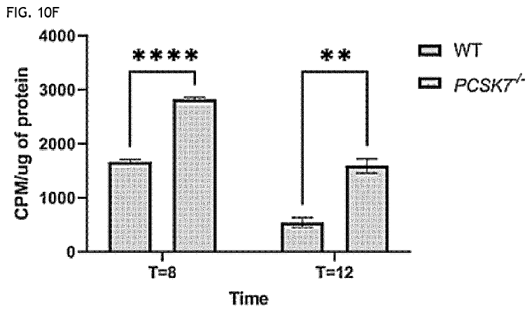

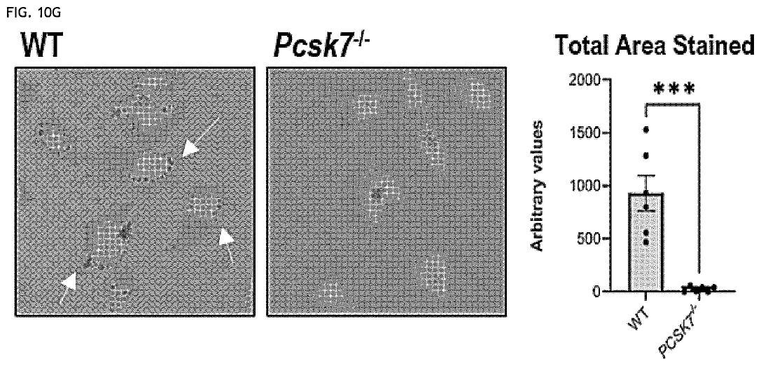

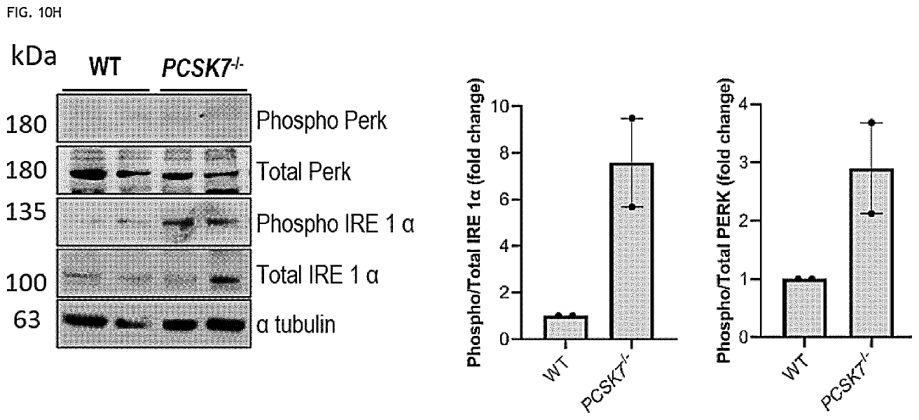

FIG. 10: Schematized GalNAc-conjugated ASOs.

### DESCRIPTION OF ILLUSTRATIVE EMBODIMENTS

The present disclosure is illustrated in further details by the following non-limiting examples.

### Example 1: Pcsk7^{-/-} Mice Exhibit Lower Circulating and Hepatic apoB, Along with Reduced Liver Lipid Accumulation

To understand the role of PC7 in vivo, the inventors generated a full body knockout of the Pcsk7 gene in mice (Pcsk7^{-/-}) (Wetsel et al., 2013).

They first investigated the levels of liver and circulating apoB in these mice.

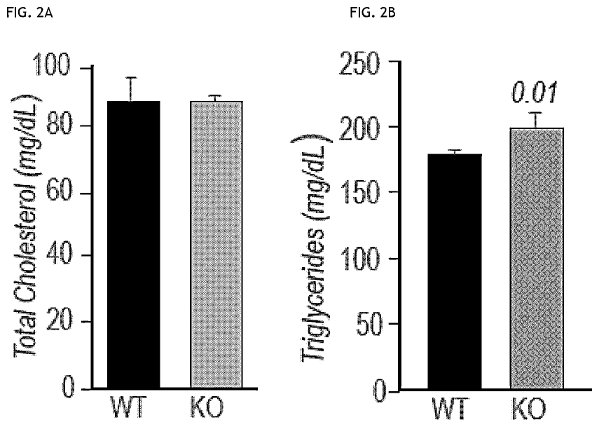

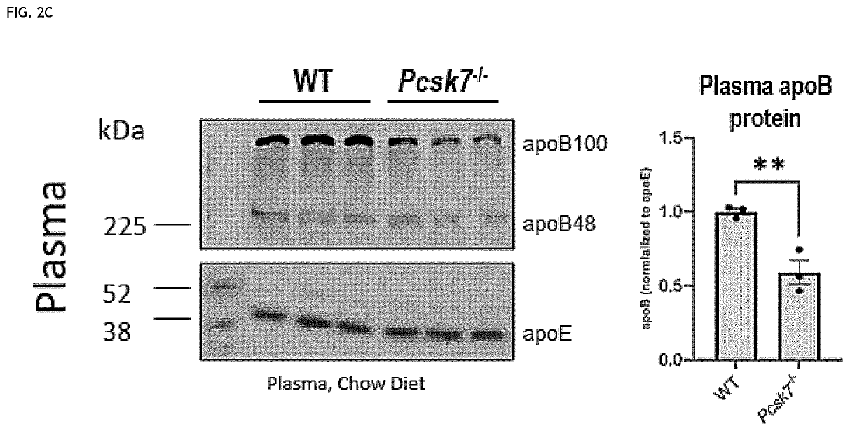

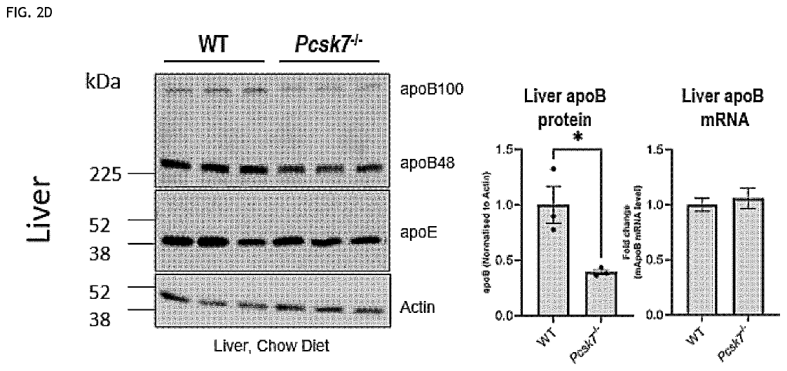

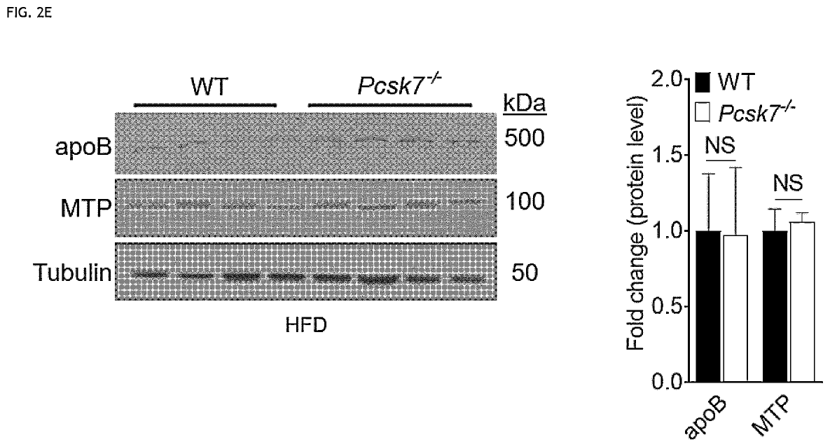

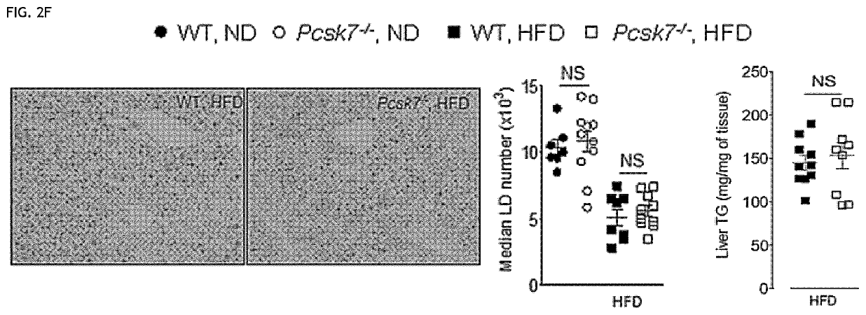

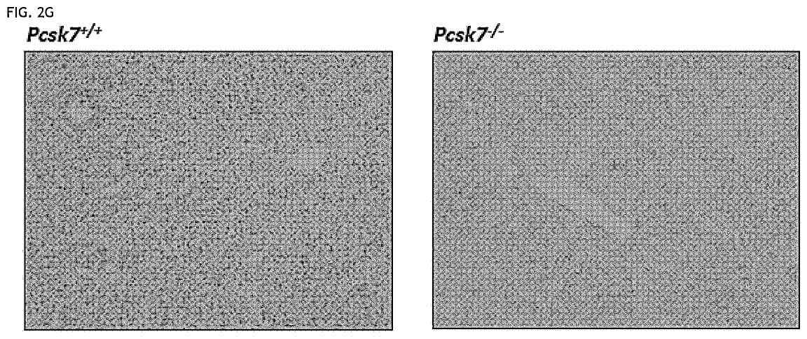

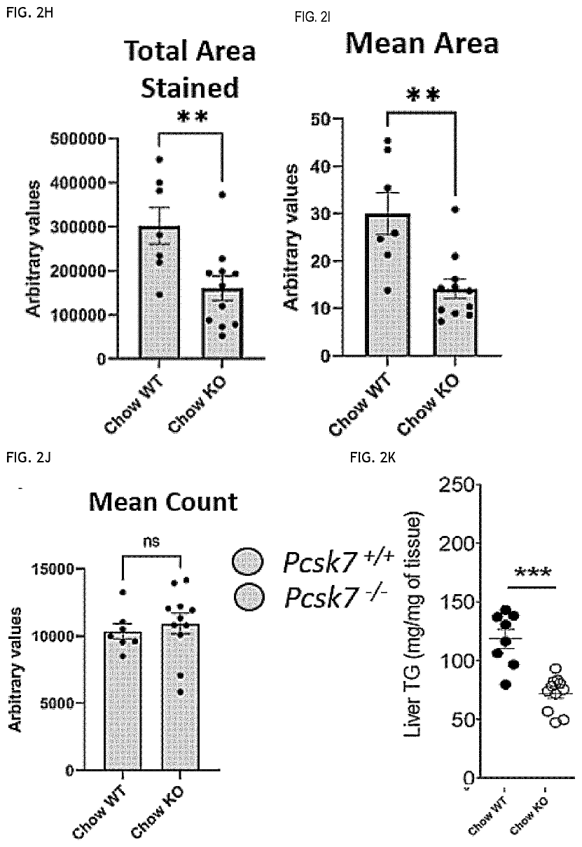

Under normal chow diet the inventors did not find any significant difference between WT and Pcsk7^{-/-} mice in circulating TC or TG (FIGS. 2A-B). Upon closer inspection, Western blot (WB) analyses revealed that mice lacking Pcsk7 exhibit a ˜50-60% reduction in circulating (FIG. 2C) and hepatic apoB100 protein levels (FIG. 2D) without any change in hepatic apoB mRNA levels (FIG. 3B) or apoB or microsomal triglyceride transfer protein (MTP) levels (FIG. 2E).

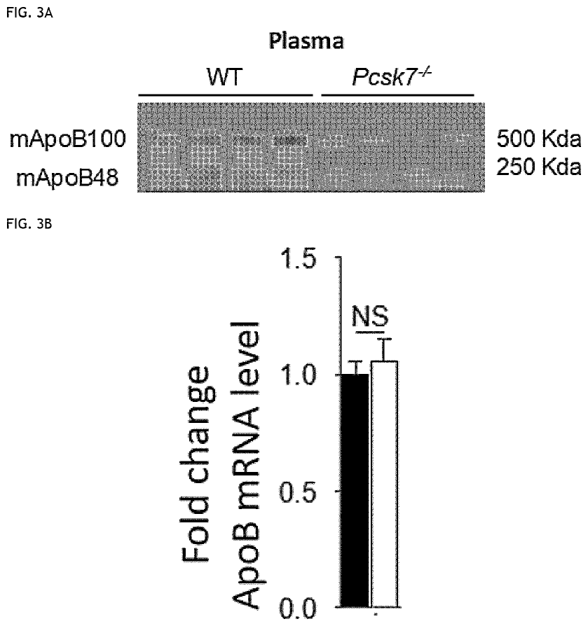

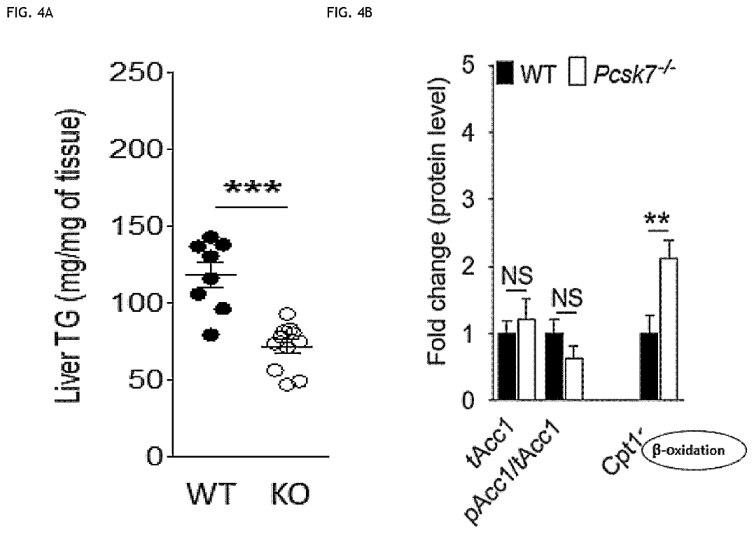

Additionally, the livers of Pcsk7^{-/-} mice showed a significant reduction in lipid droplet size (LD, TG-rich vesicles) accumulation (FIG. 2G). Image analysis revealed a ˜40-50% lower oil-red 0 staining (FIG. 2H) and mean area of LD (FIG. 2I) and a ˜30% decrease in total liver TG (FIG. 2K), with no change in total number of LD (FIGS. 2F-2J), suggesting that the absence of PC7 does not modify LD formation, but rather decreases their TG content. This is consistent with the ˜40% lower hepatic TG levels in Pcsk7^{-/-} compared to WT mice (FIG. 4A). Finally, the inventors measured in liver by Western blot (WB) the protein levels of critical proteins in lipid metabolism including total acetyl-coenzyme A carboxylase 1, Acc1 (tAcc1), the ratio of the inhibitory phosphorylated Acc1 (pAcc1c) to total Acc (tAcc1), as well as Carnitine palmitoyl-transferase 1a (Cpt1a), a rate-limiting enzyme for (β-oxidation of fatty acids (FA) (FIG. 4B). Note that protein levels of Cpt1a are ˜2.1-fold higher in Pcsk7^{-/-} mice compared to WT, suggesting that KO mice exhibit a greater potential for β-oxidation of FA.

In sum, these data suggest that lack of Pcsk7 is associated with ˜50% lower apoB levels and TG accumulation in liver.

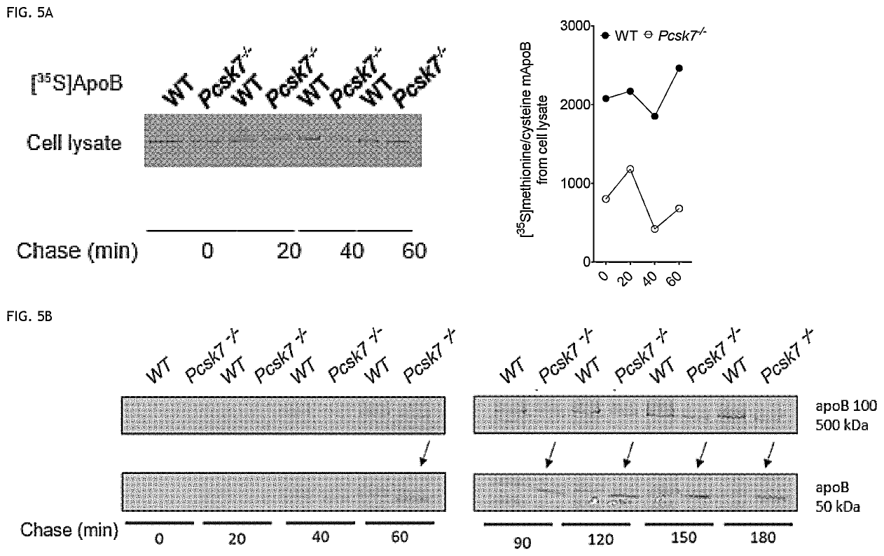

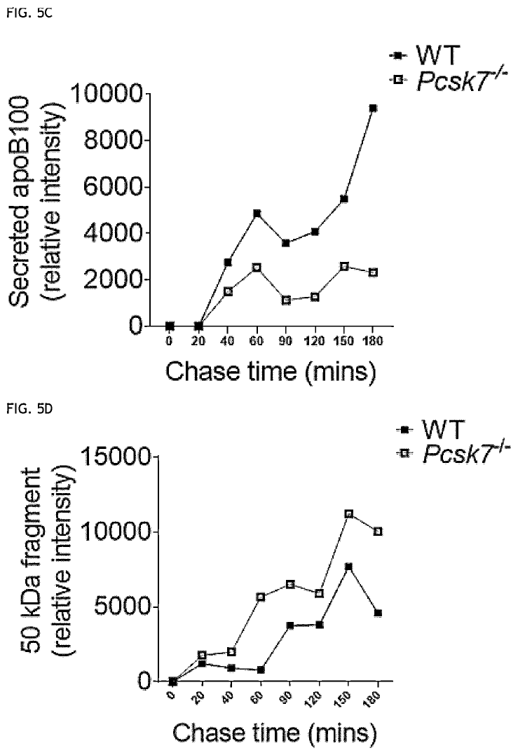

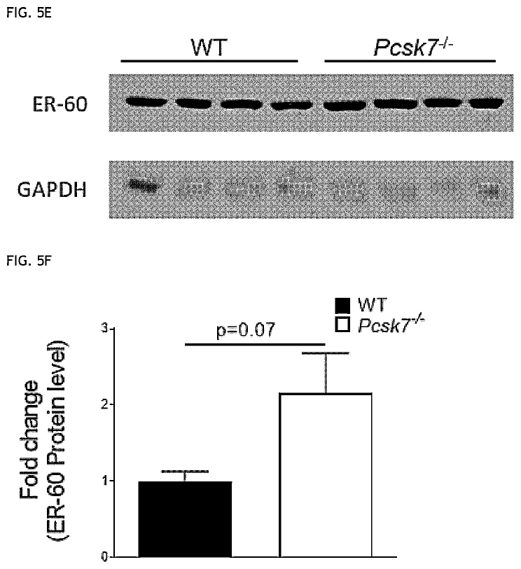

To probe the timing behind the ˜50% decreased apoB, the inventors performed a pulse-chase analysis on primary hepatocytes isolated from WT and Pcsk7^{-/-} mice. The data revealed an enhanced co-translational degradation of apoB in Pcsk7^{-/-} hepatocytes (FIG. 5A), and a reduction of apoB100 secretion associated with the increased generation of a secreted ˜50 kDa fragment of apoB in Pcsk7^{-/-} hepatocytes over chase time (FIGS. 5B-D), possibly by ER-60, an endoplasmic reticulum (ER) resident apoB quality control protease (Adeli et al., 1997). Indeed, western blot analysis revealed that the levels of ER60 proteins are 2-fold higher in Pcsk7 KO as compared to WT hepatocytes (FIGS. 5E-F). This points to the ER-associated degradation (ERAD) mechanism of apoB that is increased in absence of PC7.

### Example 2: Enhanced apoB Degradation in Cells Lacking PC7: Evidence for a Chaperone Function of PC7 Favoring apoB Secretion

Modulation of Human PC7 with siRNA

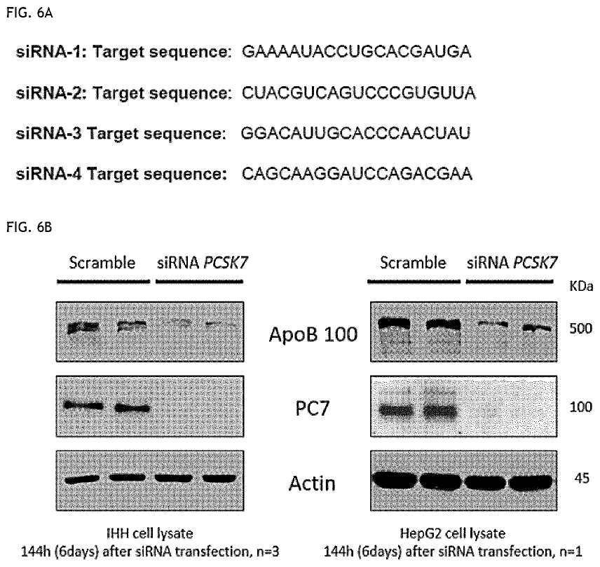

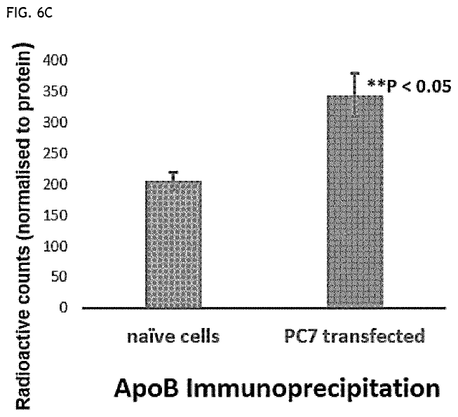

To determine whether reduced PC7 levels is associated with substantially decreased apoB secretion from human hepatocytes, the levels of endogenous apoB were analyzed in the media of human HepG2 cells and of immortalized human hepatocyte (IHH) cells, derived from immortalized human primary hepatocytes. Using a mixture of 4 optimized siRNAs against PCSK7 (Dharmacon) (FIG. 6A) the data revealed an almost complete reduction of endogenous PC7 protein levels, which was associated with a large decrease in apoB levels in the media of both cell lines (FIG. 6B). Thus, it can be concluded that in human as in mouse hepatocytes, PC7 positively regulates apoB levels.

The effect of overexpression of human PC7 also was tested on the levels of secreted triglycerides associated with ApoB which is secreted from these cells as TG-rich very low-density lipoprotein (VLDL) particles. The data show that transient transfection of human PC7 cDNA in these cells resulted in a ˜1.5-fold increased levels of secreted de novo synthesized ¹⁴C-TG within VLDL particles (FIG. 6C). Thus, overexpression of PC7 enhances the levels of secreted ApoB from hepatocytes, which is the reverse of what was observed for siRNA-mediated silencing of PC7 in IHH cells (FIG. 6B).

Silencing Human PC7 with CRISPR-Cas9

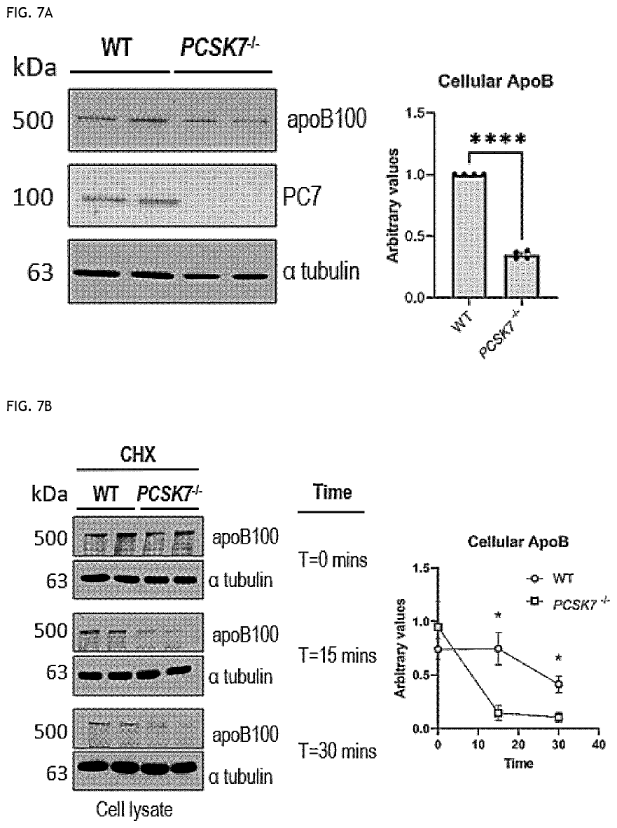

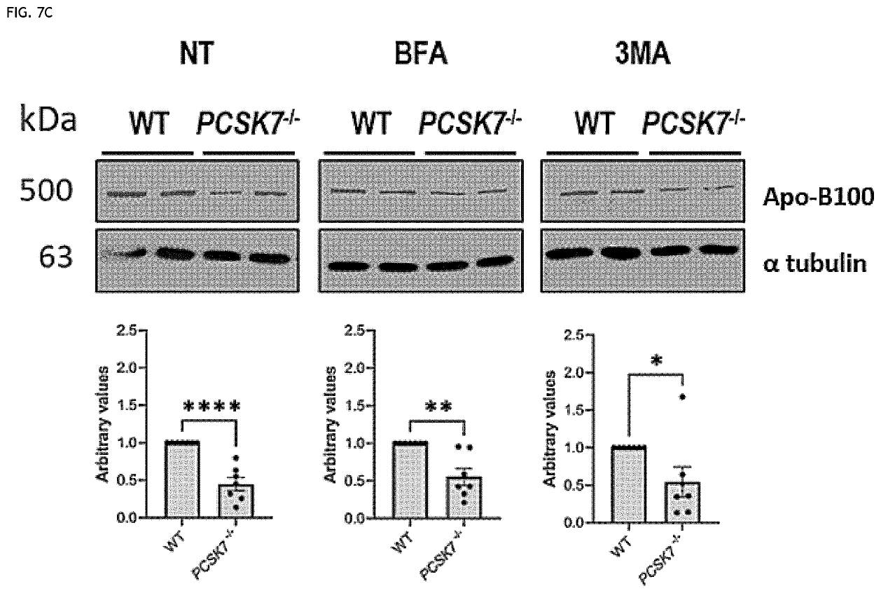

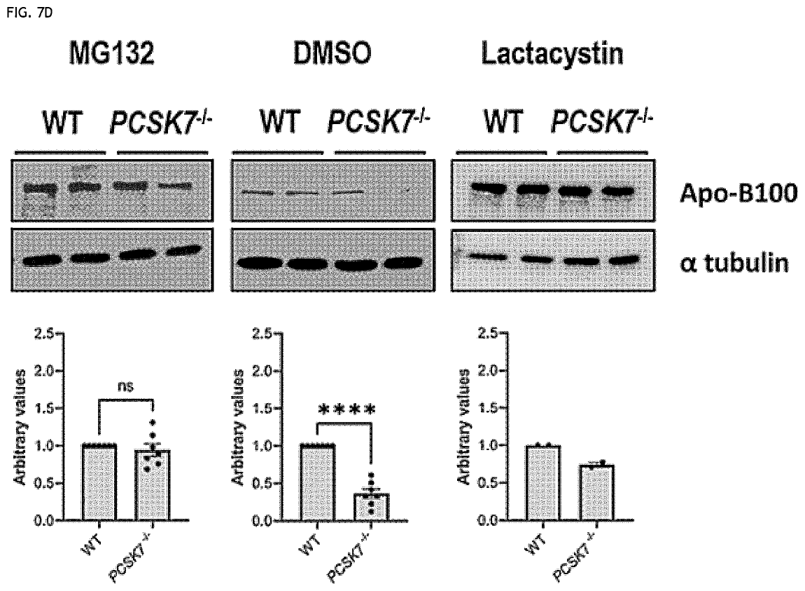

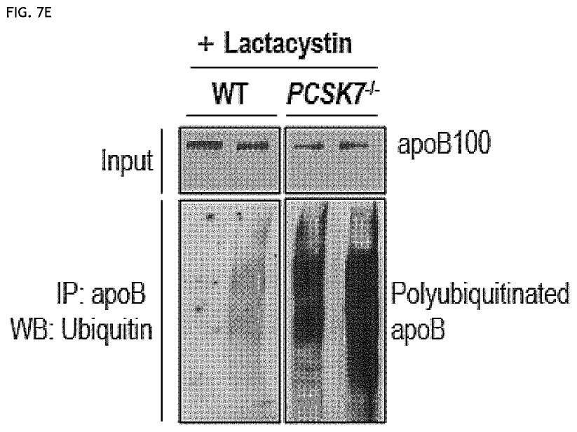

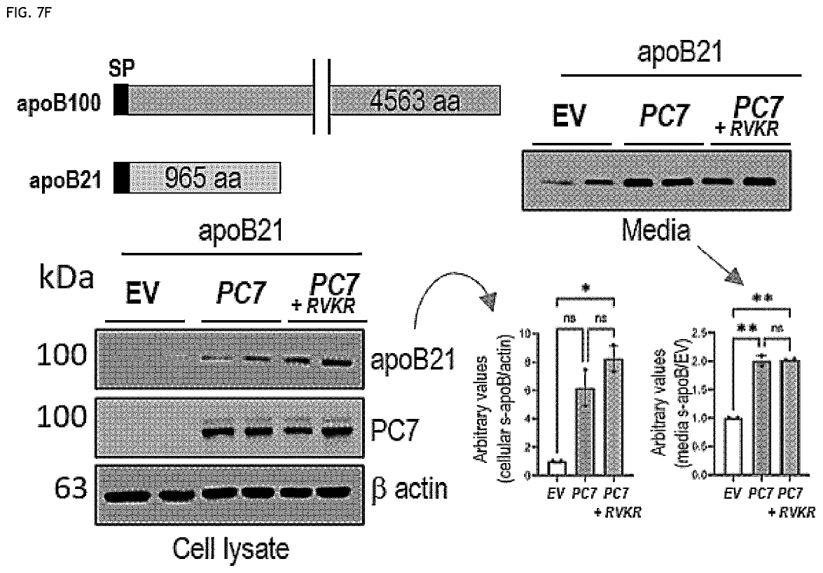

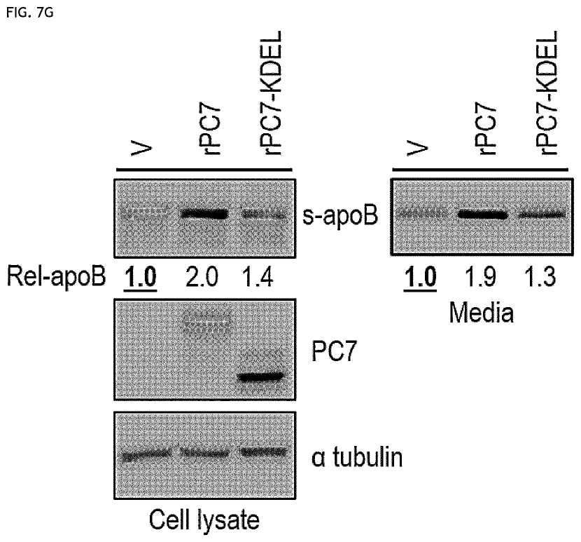

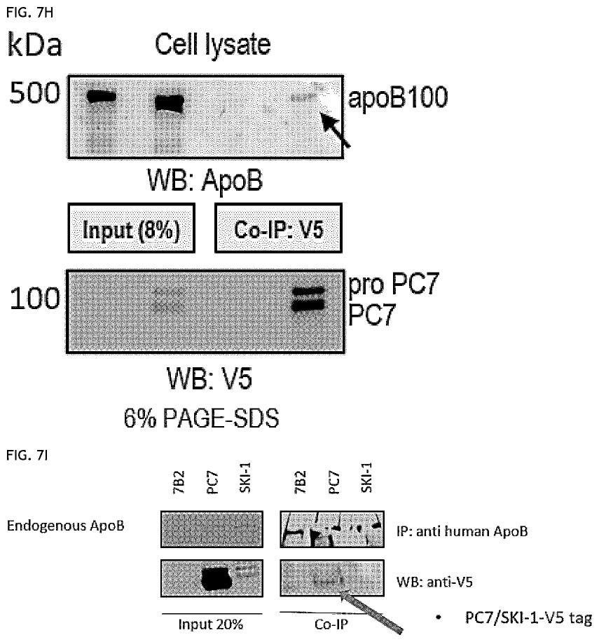

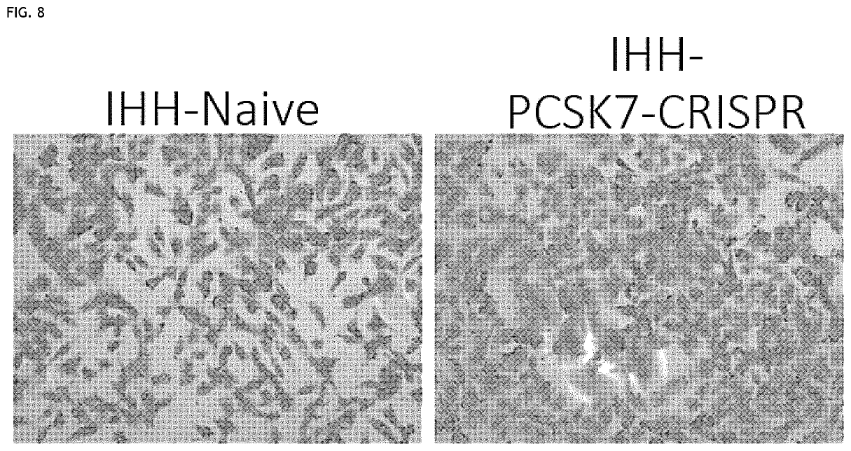

To explain the underlying mechanism of the lack of PC7 on apoB and hepatic TG levels in mice and to extend this observation to human cells, PCSK7 expression was deleted using CRISPR-Cas9 from IHH cells by incubating CRISPR-mediated knockout PCSK7 IHH cells with oleate for 72 h. As previously observed in mice, the lack of human PC7 (PCSK7^{-/-} cells) resulted in ˜60% reduction in endogenous intracellular apoB100 protein levels (FIG. 7A). It reduced level & size of intracellular lipid droplets (revealed by oil-red-0 labeling) and enhanced cell proliferation based on cell count compared to control IHH cells (FIGS. 8, 10G)

To define the time course of apoB degradation, these IHH cells were incubated with cycloheximide (CHX) to block de novo mRNA translation. The data show that PCSK7^{-/-} cells exhibit a much faster post-translational degradation of apoB, with a major reduction occurring within the first 15 min after CHX addition (FIG. 7B), in agreement with the early apoB100 degradation observed in primary hepatocytes following a 15 min pulse (FIGS. 5A-B).

To identify the degradation pathway, WT and PCSK7^{-/-} IHH cells were incubated with either brefeldin A (BFA) that blocks exit from the endoplasmic reticulum (ER), an autophagy inhibitor 3-methyladenine (3MA), proteasome inhibitors MG132 or Lactacystin and compared cellular apoB levels to DMSO or non-treated (NT) controls. The data clearly point to an enhanced co-translational early proteasomal degradation (MG132 and Lactacystin) of apoB in absence of PC7 (FIGS. 7C-D), as confirmed by increased polyubiquitination of apoB20 (FIG. 7E) and absence of BFA inhibition (FIGS. 7C-D).

Since PC7 is a secretory serine protease related to yeast kexin, naïve IHH cells overexpressing PC7 and a secretory short N-terminal fragment of apoB (s-apoB; aa 1-965 aa) were incubated with a cell permeable pan-proprotein convertase inhibitor decanoyl-RVKR-cmk, that blocks PC7 shedding activity of TfR1 (Guillemot J et al, 2013). Notably, overexpression of PC7 enhanced the secretion of s-apoB (apoB21) suggesting that PC7 binds an N-terminal domain of apoB, likely resulting in a “chaperone-like” effect preventing apoB degradation and enhancing its exit from the ER and secretion into the medium. Additionally, decanoyl-RVKR-cmk did not block this chaperone-like activity of PC7 on apoB, suggesting it occurs via a non-enzymatic pathway (FIG. 7F), as was also observed for apoA-V10 and the major histocompatibility complex-I (MHC-I) (PMID: 20164418).

The inventors next examined the possibility that PC7 may bind apoB in the ER. Accordingly, rat PC7 and its soluble-KDEL variant (retained in the ER) were co-expressed in naive IHH cells with s-apoB. The data showed that like human PC7, rat PC7 (rPC7) also enhanced by ˜2-fold the secretion of s-apoB (apo21). However, rPC7-KDEL significantly reduced (˜30%) s-apoB (apo21) secretion compared to rPC7 likely by retaining it in the ER (FIGS. 7G, 6C). In support of this observation, in IHH cells overexpressed PC7 specifically co-immunoprecipitated with endogenous apoB100 (FIG. 7H). Co-immunoprecipitation (co-IP) experiments also showed that endogenous ApoB from IHH cells does co-IP with membrane-bound PC7 (tagged with V5), but not with a control membrane-bound convertase SKI-1 (tagged with V5) (Abifadel et al., 2003) (FIG. 7I).

The above data revealed that PC7 can bind apoB in the ER and acts as a molecular chaperone-like facilitating its secretion, and its absence leads to a rapid degradation of apoB by the proteasome.

### Example 3: Mechanism of Hepatic TG Reduction in Pcsk7^{-/-} Mice and Human IHH Cells

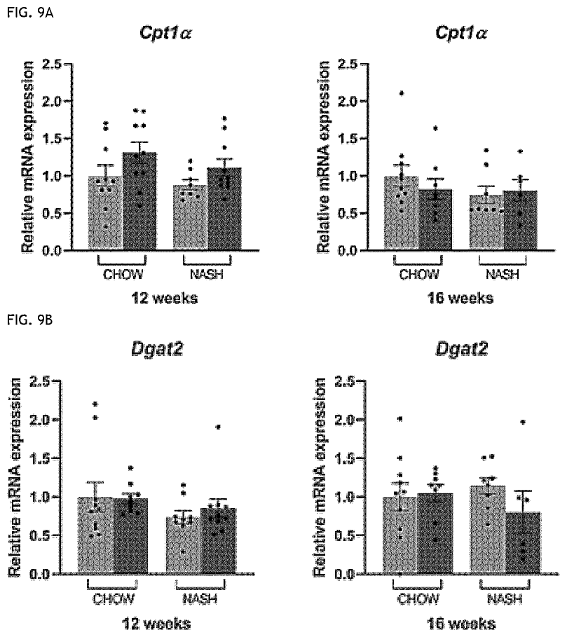

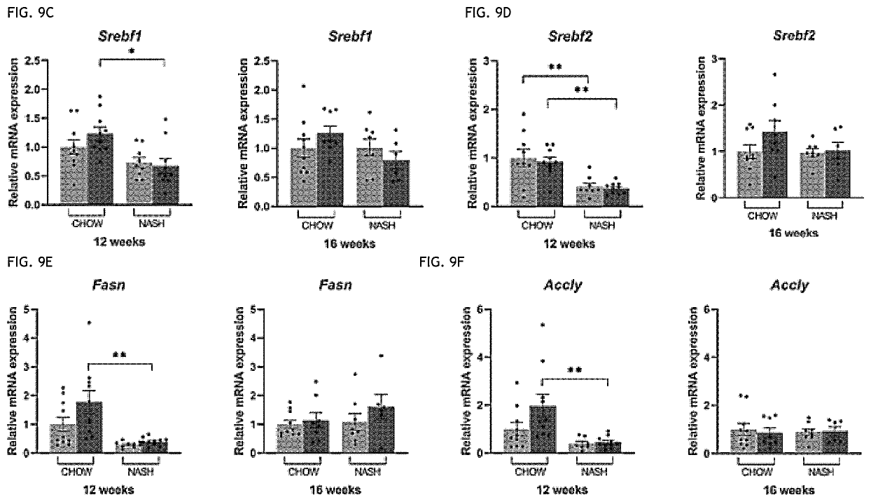

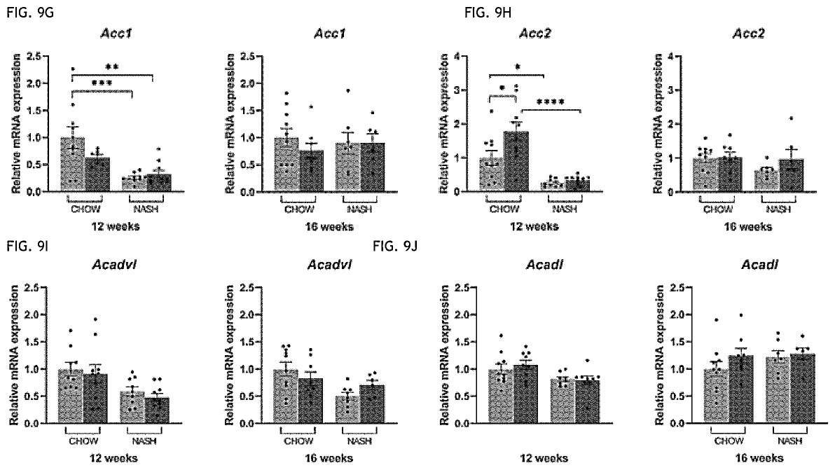

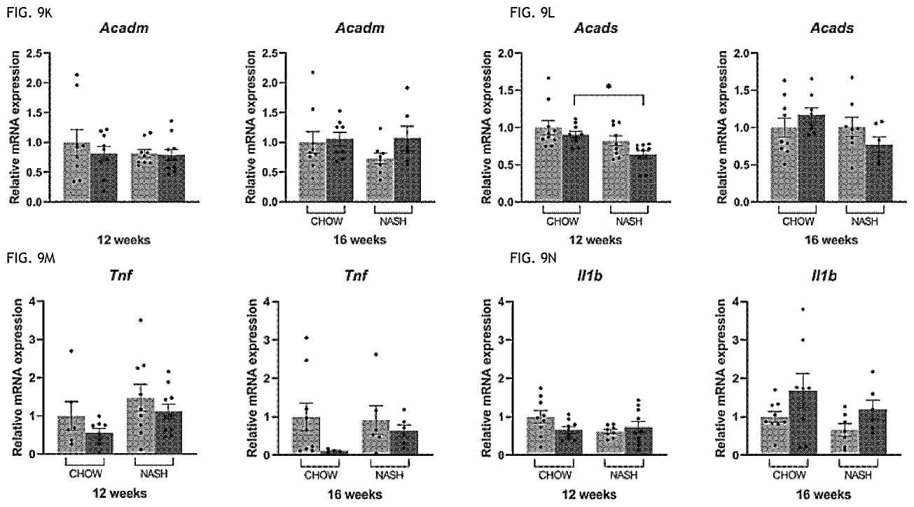

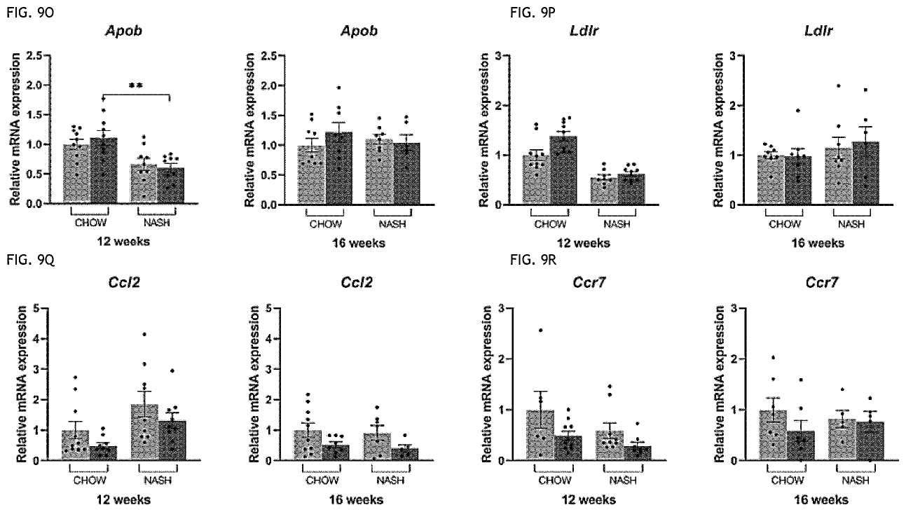

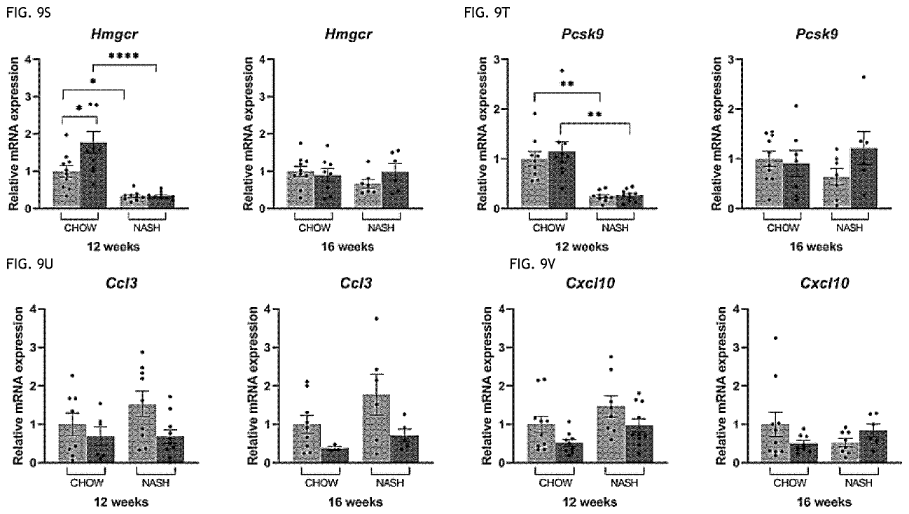

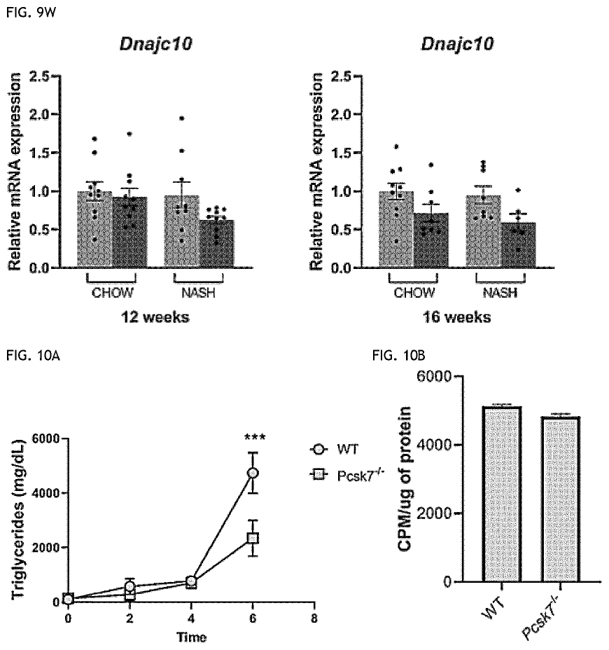

To probe for the mechanism behind the reduction of hepatic TG in Pcsk7^{-/-} mice, the inventors extensively analyzed mRNA levels of 23 liver genes known to be involved in fatty acid (FA) oxidation, esterification, lipogenesis, inflammation, and cholesterol metabolism. However, the inventors did not detect any significant mRNA expression differences (FIGS. 9A-W).

According to the literature, lower hepatic accumulation of lipids observed in Pcsk7^{-/-} mice (FIGS. 2G-J) could be due to many factors: (1) increased secretion of TG-enriched Very-low-density lipoprotein (VLDL); (2) decreased de novo synthesis of FA; (3) reduced levels of albumin-bound non-esterified FA (NEFA) in circulation that would be uptaken by the liver and converted into TG; (4) reduced levels of liver receptors implicated in uptake of TG-enriched lipoproteins; (5) increased lipophagy; or (6) enhanced β-oxidation of FA. The inventors address each of these possibilities hereinbelow.

To determine whether increased hepatic VLDL secretion is causing the observed reduction of LD in liver (FIGS. 2GC-J), plasma TG levels were measured in vivo following administration of the detergent poloxamer-407 (P-407), which blocks TG-lipolysis. At 6 h after injection of P-407 the plasma TG levels were ˜50% lower in Pcsk7^{-/-} mice (FIG. 10A), revealing a significantly reduced hepatic secretion of VLDL-TG in Pcsk7^{-/-} mice, thereby eliminating possibility #1. To look for changes in de novo lipogenesis, primary hepatocytes of WT and Pcsk7^{-/-} mice were incubated with 3H-acetate for 2 h, the 3H-TG were extracted, and the de novo incorporated radioactivity counted, as reported (PMID: 26382148). Clearly, possibility #2 was also discounted, as no significant difference in de novo lipogenesis between the two genotypes could be detected (FIG. 10B). Pyruvate kinase (PKLR), liver receptors for TG-enriched VLDL (LDL receptor-related protein 1, LRP1) and albumin-bound fatty acids (CD36), and plasma non-esterified free FA (NEFA) (FIGS. 10C-D) are unchanged in both genotypes, eliminating possibilities #3 and 4. In contrast, possibility #5 is plausible, since the inventors observed a significant increase in functional macroautophagy-lysosome pathway in the primary hepatocytes of Pcsk7^{-/-} mice, whereby the levels of LC3-II are higher both in absence and presence of chloroquine (FIG. 10E), suggesting enhanced functional lipophagy, also reported previously in absence of apoB (PMID: 27599291).

To address possibility #6, the inventors compared the FA oxidation potential in human hepatic IHH cells expressing or completely lacking PCSK7 (IHH-CRISPR PCSK7 KO). Using a published protocol (PMID: 26382148), the cells were incubated with ¹⁴C-oleate and the released ¹⁴CO₂ was captured at different time points, thereby revealing enhanced FA β-oxidation (increased ¹⁴CO₂) both after 8 h and 12 h from the start of the incubation (FIG. 10F).

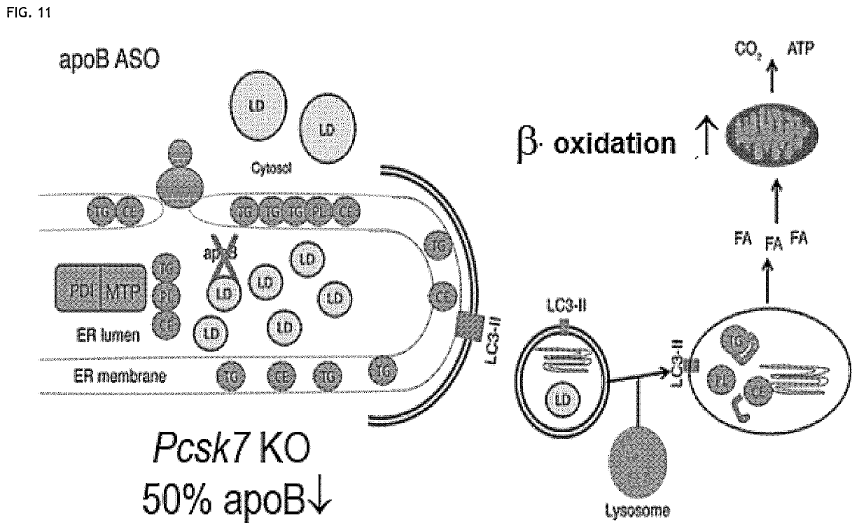

The inventors also analyzed by western blot the protein levels of critical hepatic regulators implicated in de novo ß-oxidation. Under normal diet, the protein levels of Cpt1a were ˜2.1-fold higher in Pcsk7^{-/-} mice compared to WT, further suggesting that KO mice exhibit a greater potential for ß-oxidation (FIG. 4B). Thus, it is concluded that absence of PC7 results in enhanced degradation of apoB by the ERAD pathway, which ultimately leads to enhanced ER-associated autophagy and hydrolysis of TG, ultimately leading to fatty acid release and their enhanced ß-oxidation in mitochondria (model FIG. 11).

To extend the observation of reduced LD accumulation in the liver of Pcsk7^{-/-} mice (FIGS. 2G-J) to human cells, the inventors incubated overnight IHH and IHH-CRISPR PCSK7 KO cells with 0.3 mM oleic acid (OA) and then washed the cells and incubated them in medium lacking OA for 88 h. The cells were then stained with Oil-red O revealing the loss of LD-staining in absence of PCSK7 (FIG. 10G), as observed Pcsk7^{-/-} mice.

Since they hypothesized that PC7 is a chaperone-like for apoB (Example 2), and as the absence of mouse apoB was reported to enhance ER stress (PMID: 27599291), they compared the protein levels of ER-stress markers in IHH versus IHH-CRISPR PCSK7 KO cells. The data showed higher levels of phospho-IRE1c and phospho-PERK in cells lacking PCSK7 (FIG. 10H), suggesting an activated unfolded protein response (UPR) signaling in absence of PCSK7.

In sum, the upregulated UPR signaling connects the observation of lower apoB to the loss of the potential apoB chaperone PC7, and to lower lipid accumulation in hepatocytes.

### Example 4: Mechanism of TG Level Variation in Pcsk7^{-/-} Mice

To understand the mechanism behind PC7-induced changes in TG levels, primary hepatocytes were isolated from WT and KO mice, as previously described (Essalmani et al., 2013). These cells were then incubated with radiolabeled ¹⁴C-oleic acid, TG content was analyzed at different time points (0, 6 h, 12 h, 24 h and 36 h) by lipid extraction and separation on thin layer chromatography plates. The data showed that primary hepatocytes isolated from Pcsk7^{-/-} mice accumulate much less ¹⁴C-oleic acid (fat) compared to WT (FIG. 12A), consistent with the reduced TG levels observed in livers of KO mice (FIG. 4A).

To determine whether lack of PC7 results in lower synthesis of lipids, the inventors performed a de novo lipogenic assay by incubating primary hepatocytes with ¹³C-acetate and measuring lipid synthesis. The data revealed no change in lipogenesis (FIG. 12B), suggesting that the lower TG observed in Pcsk7 KO livers may be due to enhanced lipid metabolism (fat burning) rather than reduced synthesis.

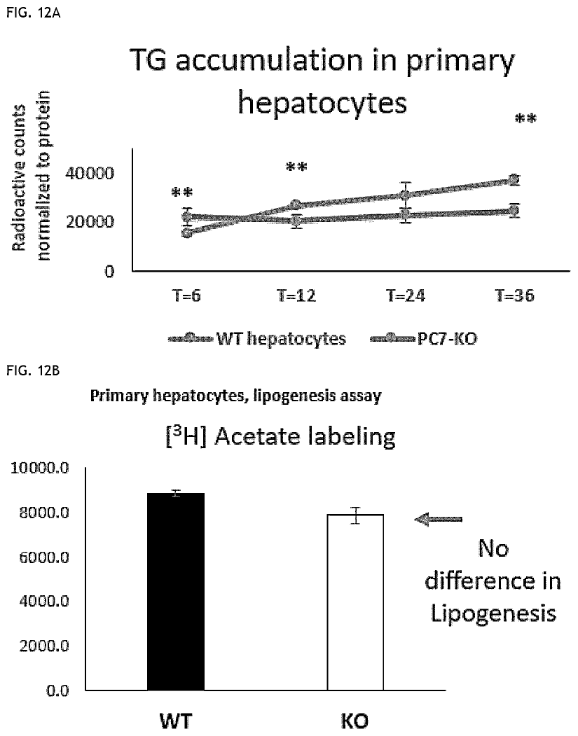

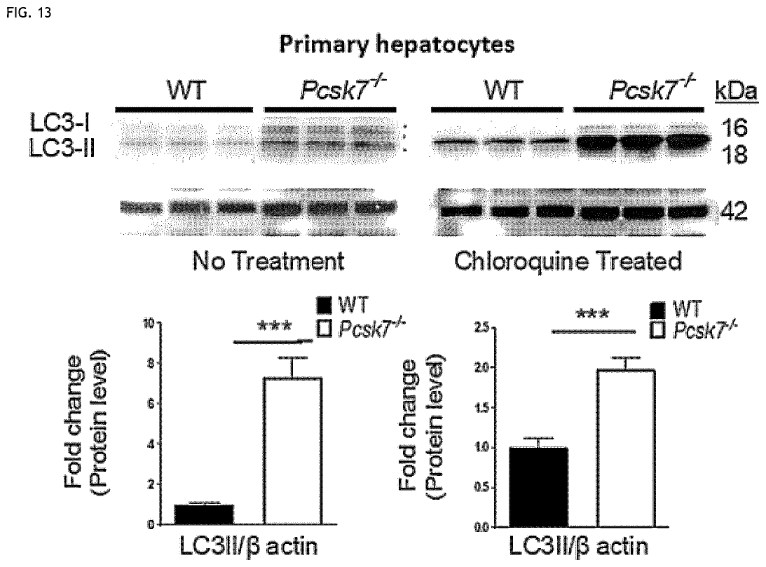

To further probe this fat-burning mechanism, the inventors measured the levels of the autophagosomal marker micro-tubule-associated protein 1A/1B-light chain 3 (LC3-II) by western blot in primary hepatocytes isolated from WT and Pcsk7^{-/-} mice. Amazingly, the data showed that LC3-II levels are ˜6 to 7-fold higher in the Pcsk7^{-/-} primary hepatocytes compared to WT (FIG. 13).

Additionally, treatment with chloroquine, a lysosomal inhibitor, showed accumulation of LC3-II in both WT and Pcsk7-hepatocytes, with a greater accrual in the latter, indicating that the surge in LC3-II is associated with an increased biogenesis of autophagosomes. It is concluded that under normal diet, the lack of PC7 leads to enhanced ER-associated autophagy and hydrolysis of TG, ultimately leading to fatty acid release and their enhanced ß-oxidation in mitochondria, as reported in mice treated with an antisense oligonucleotide targeting apolipoprotein B (apoB)(Conlon et al., 2016).

### Example 5: Liver Fat Accumulation in WT Versus Pcsk7^{-/-} Mice Recovering from a High Fat/Sugar(Fructose)/Cholesterol (HFFC) Diet

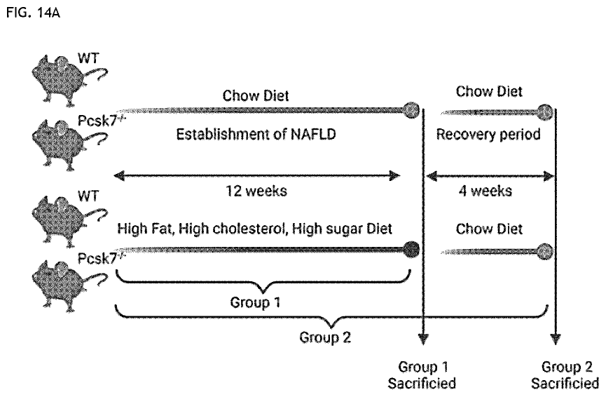

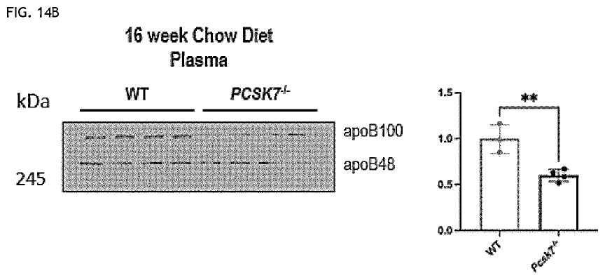

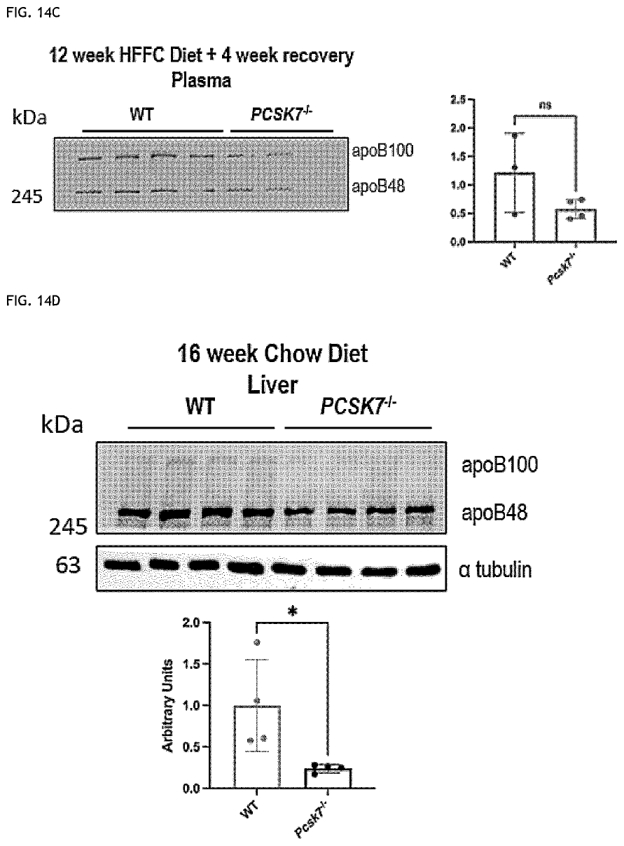

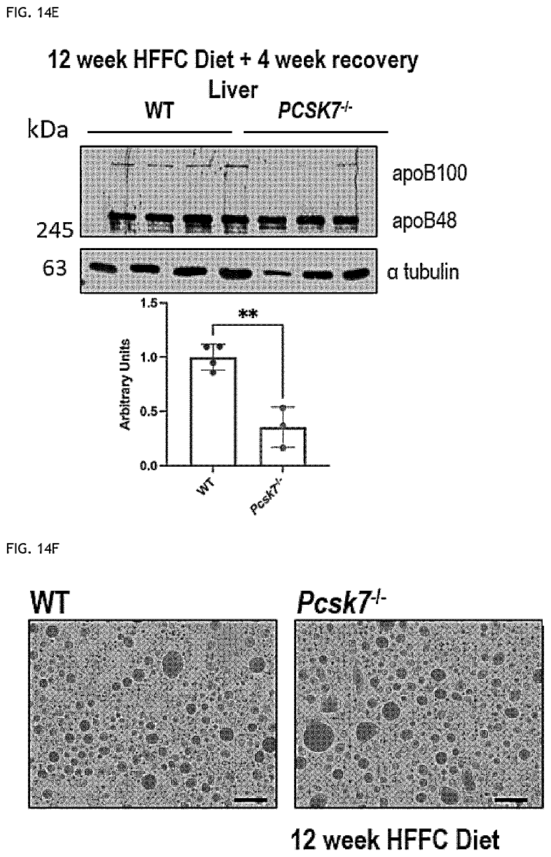

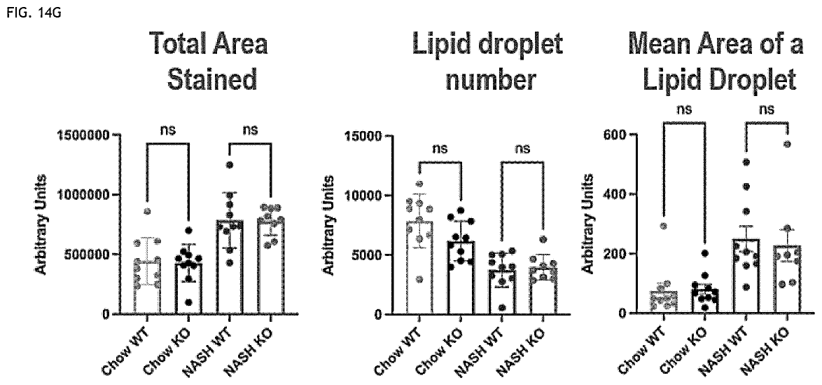

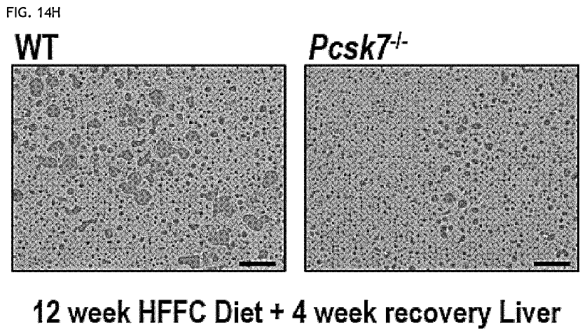

The inventors next probed the effect of the loss of PC7 expression on the extent fat accumulation in the liver, following a Western diet high in fat, fructose, and cholesterol (HFFC), reported to induce a robust NAFLD-like phenotype in mice (PMID: 32924526). Accordingly, four groups of 20 WT and 20 Pcsk7^{-/-} mice (2-month-old) were fed a chow or HFFC diet for 12-weeks, after which half of them were sacrificed for assessment of the development of NAFLD, and the other half were allowed to recover under a chow diet for another 4 weeks (FIG. 14A). This recovery strategy was elaborated based on the inventors' evidence that no change in LD or apoB were observed under high fat diet (HFD) (FIGS. 35C-D). Indeed, Western blot analyses revealed that the pre-recovery apoB levels were unchanged at the 12-week period in plasma and liver (FIGS. 15A-B). In contrast, apoB levels were reduced at the end of 16-week under chow diet or post-recovery following HFFC diet in plasma (FIGS. 14B-C) and liver (FIGS. 14D-E). The data show that in both plasma and liver apoB100 levels are ˜50-60% lower in Pcsk7^{-/-} mice compared to WT ones, with no significant change in apoB48, in agreement with those shown in FIGS. 2C-D.

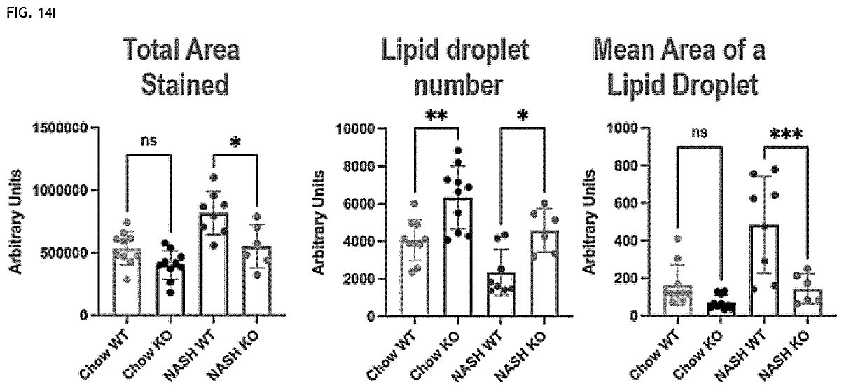

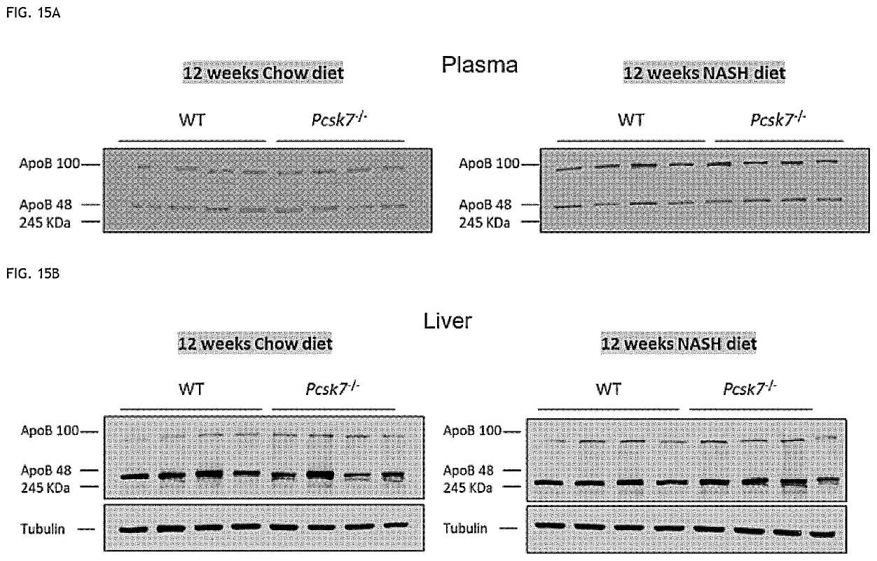

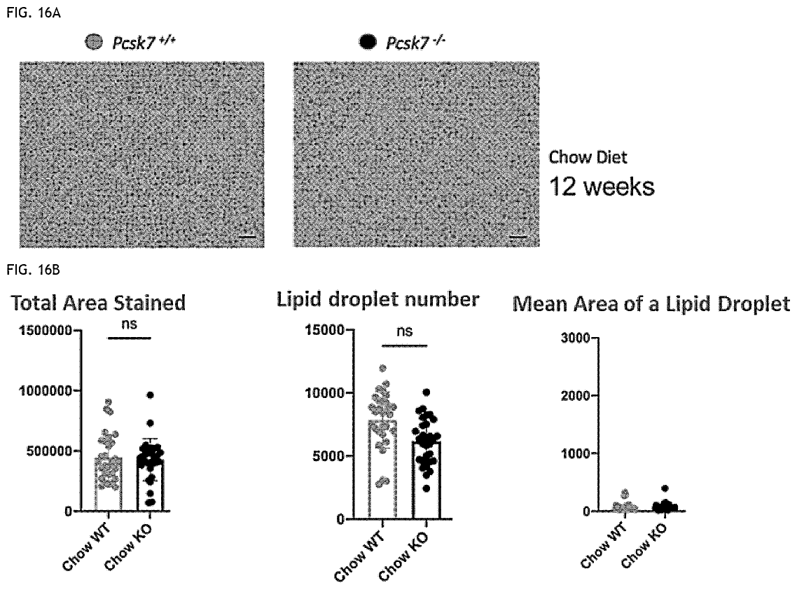

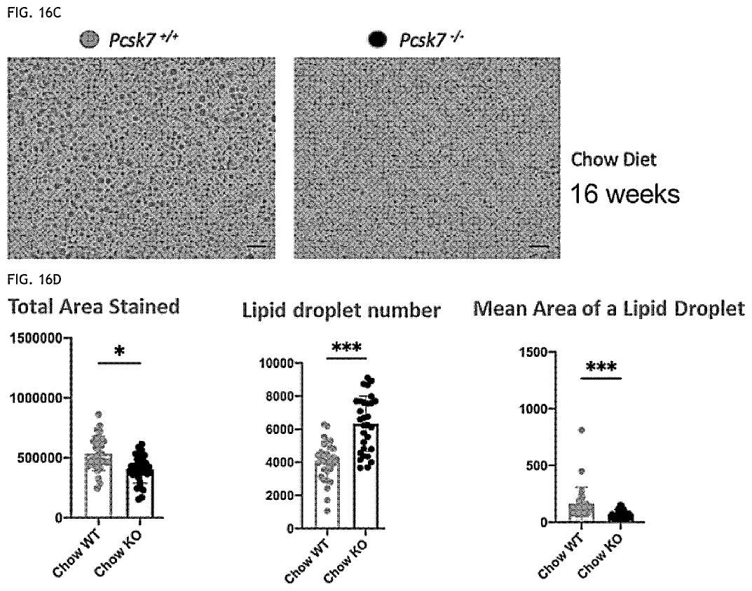

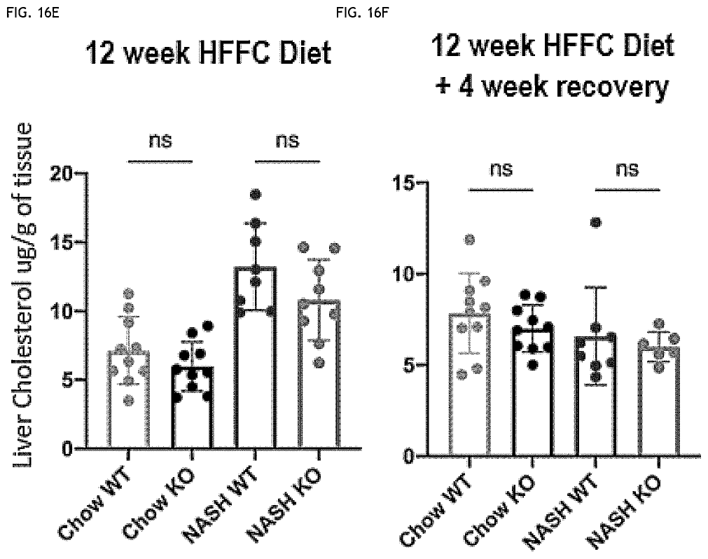

Oil red O (ORO) staining of liver sections revealed a robust accumulation of LD following the HFFC diet in both genotypes, suggesting the lack of PC7 does not prevent the occurrence of diet induced NAFLD (FIGS. 14F-G). In contrast, following the recovery period the absence of PC7 dramatically reduced the extent of LD accumulation, evidenced by the reduced total area stained by ORO, decreased mean area, and increased number of LD (FIGS. 14H-I). This suggests that the lack of PC7 is associated with a higher metabolism of LD into smaller ones, as also observed in human IHH cells (FIG. 10G). It should be noted that 16-week-old mice fed a chow diet exhibited a significant ˜20% reduction in LD in Pcsk7^{-/-} mice versus WT (FIGS. 16C-D).

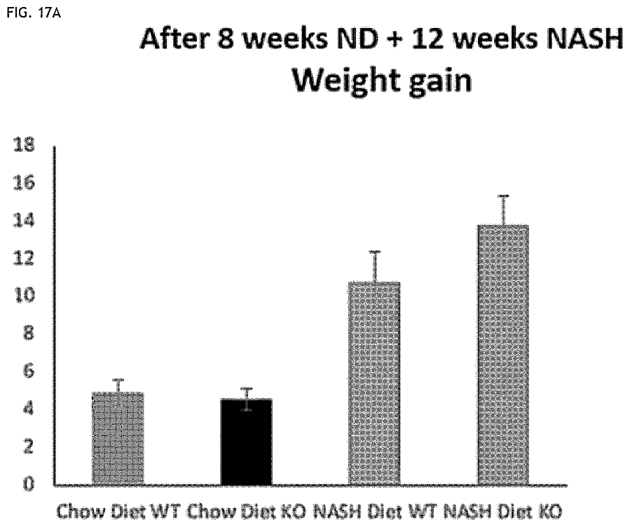

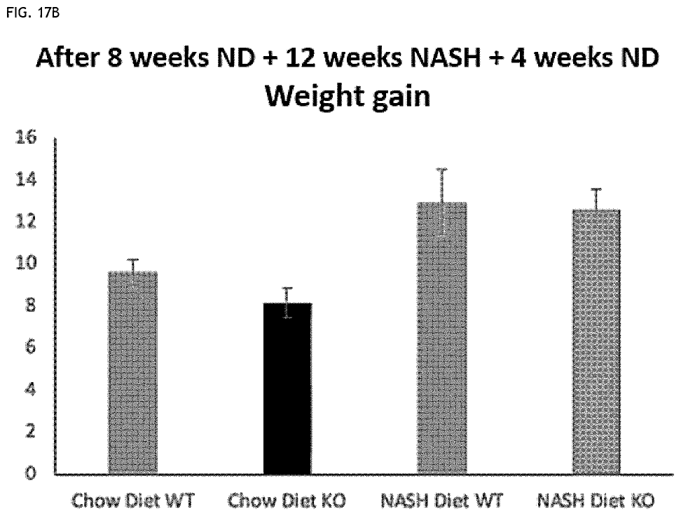

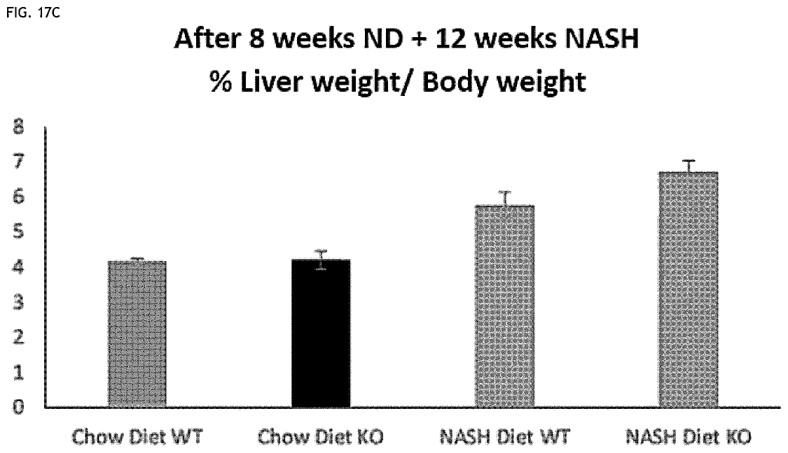

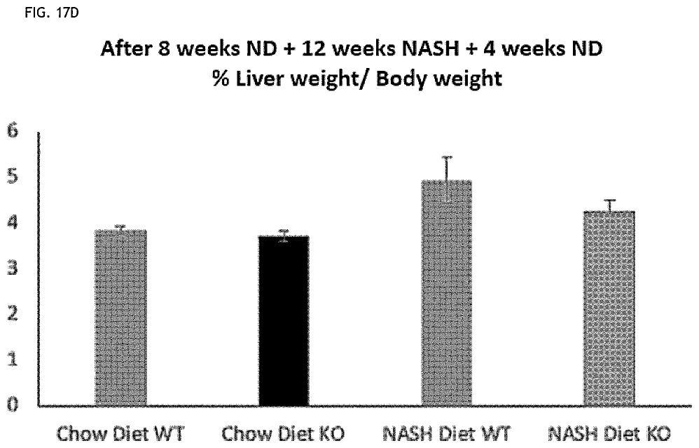

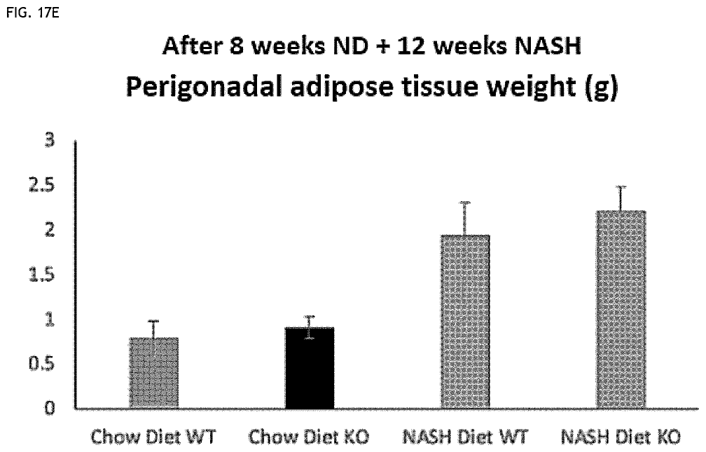

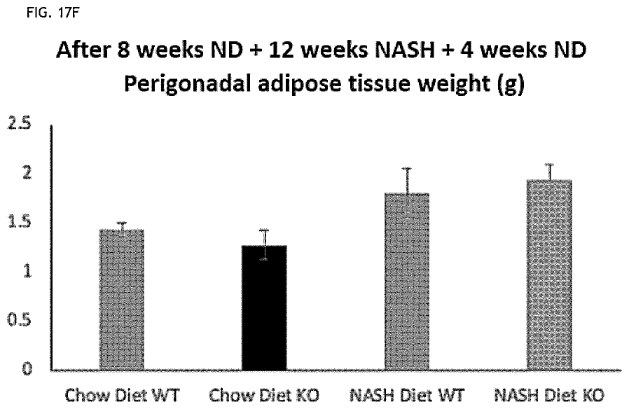

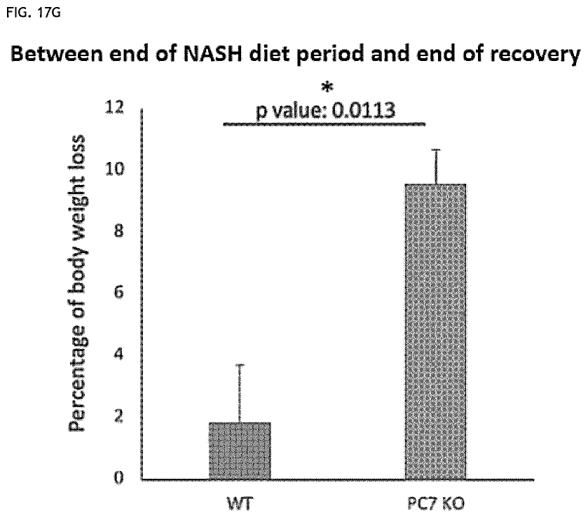

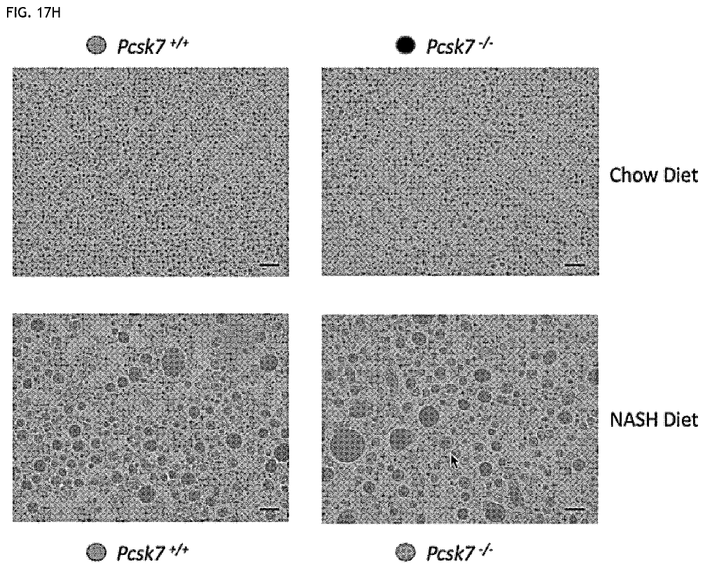

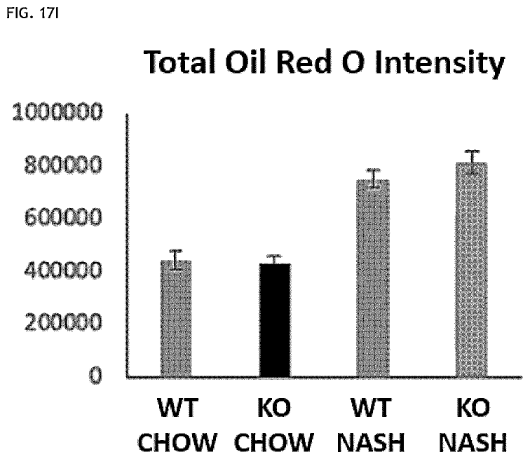

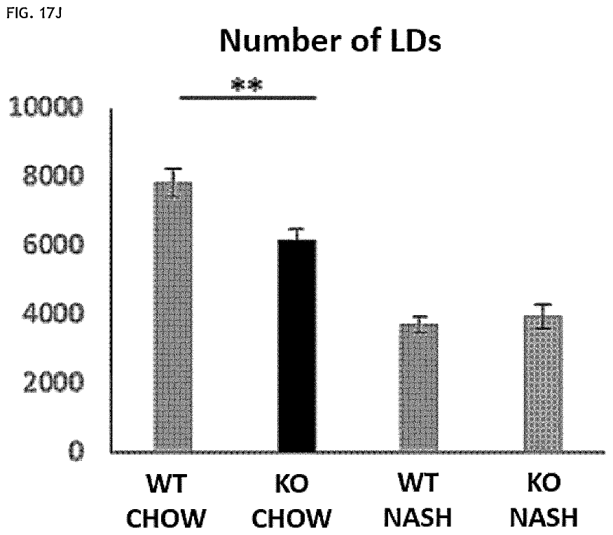

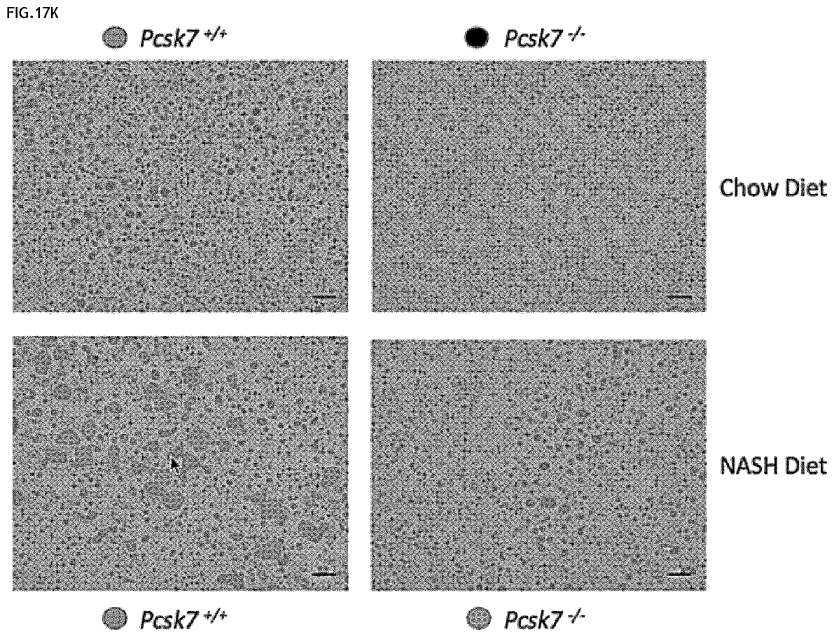

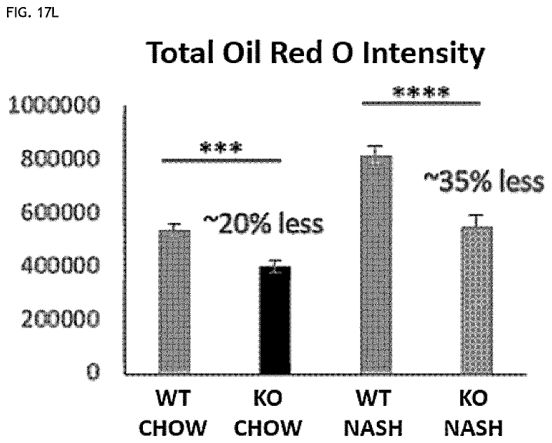

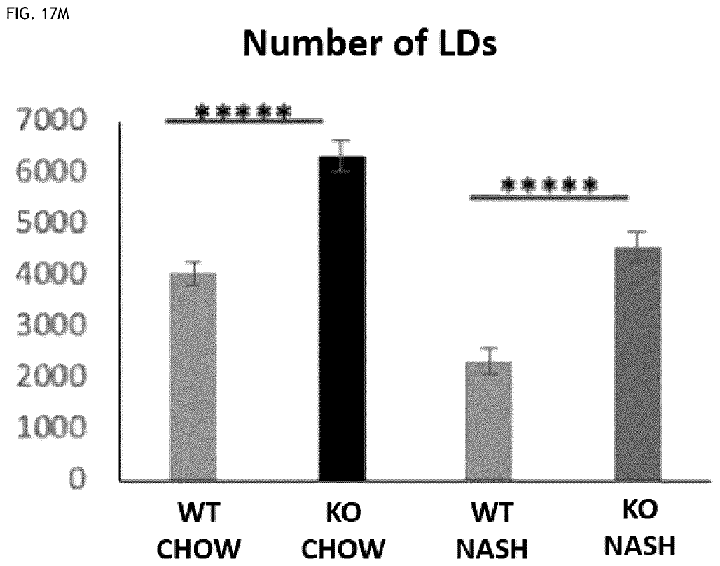

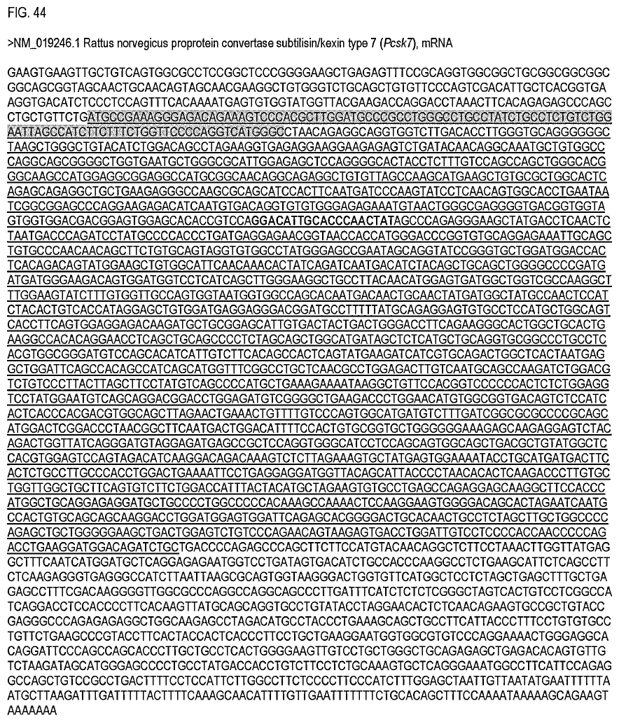

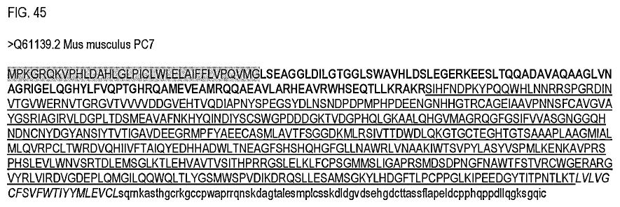

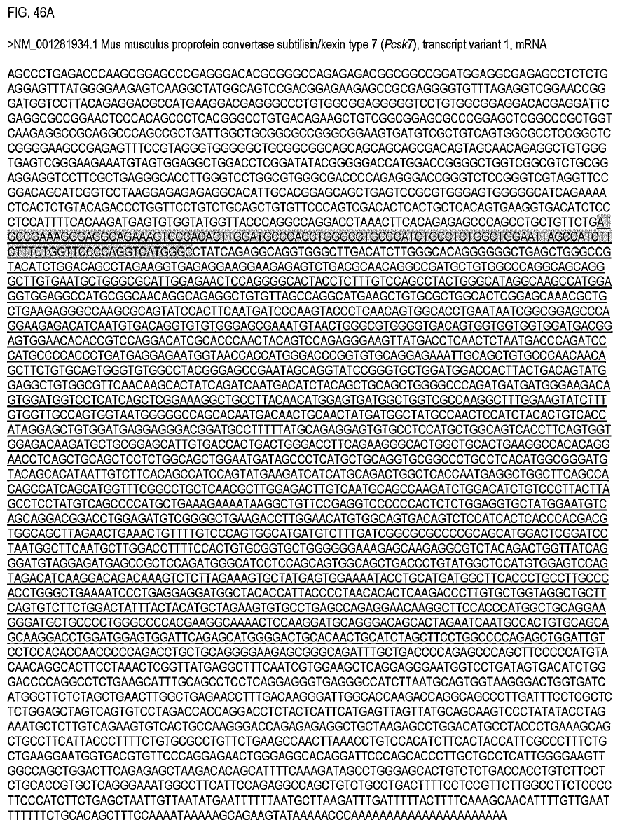

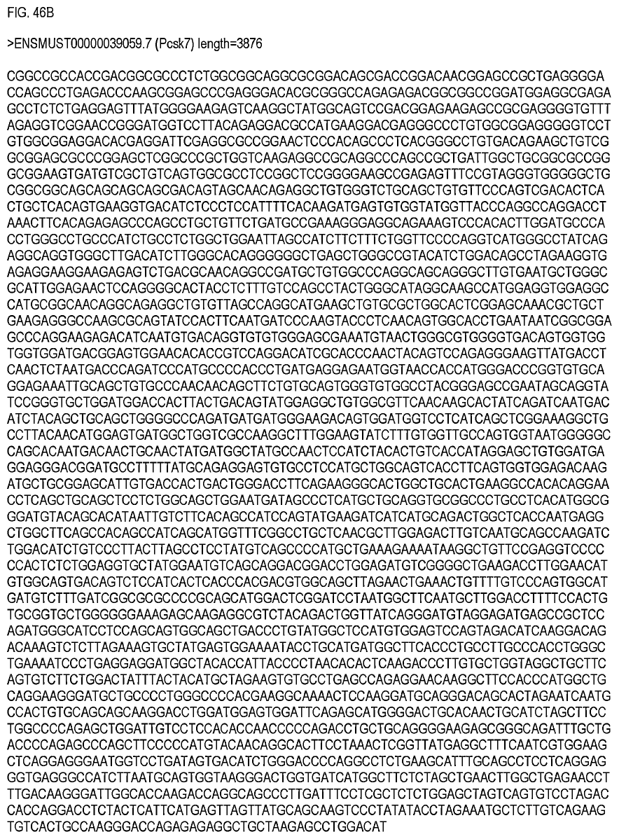

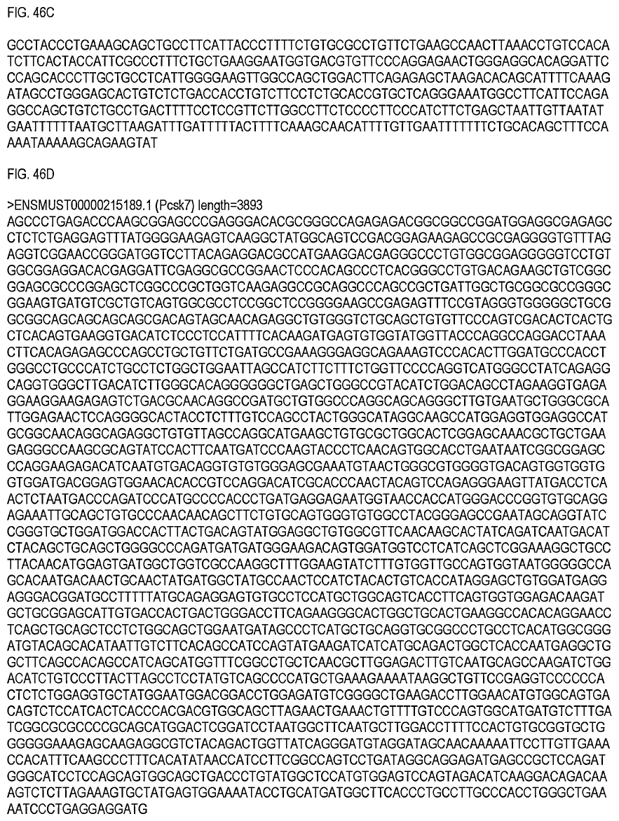

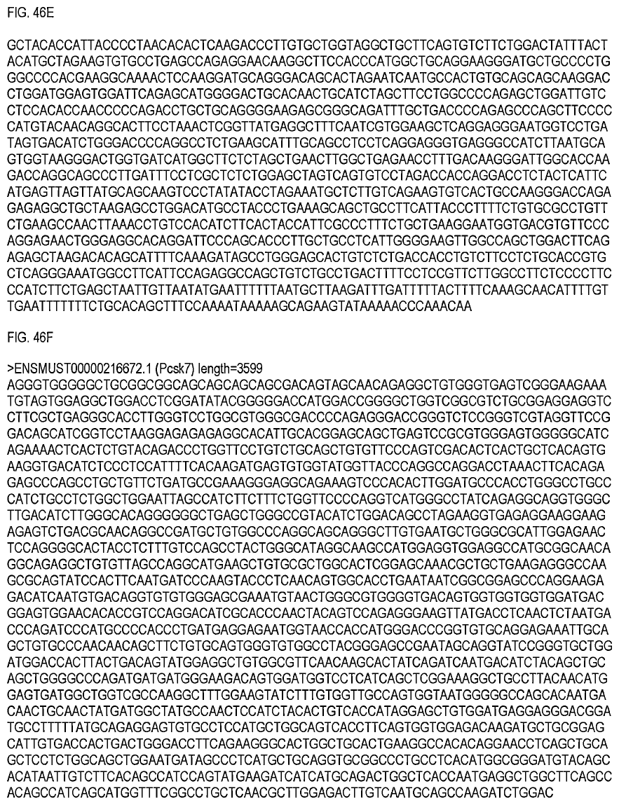

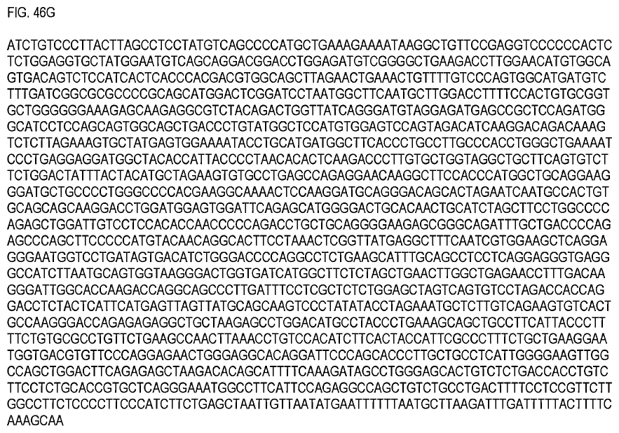

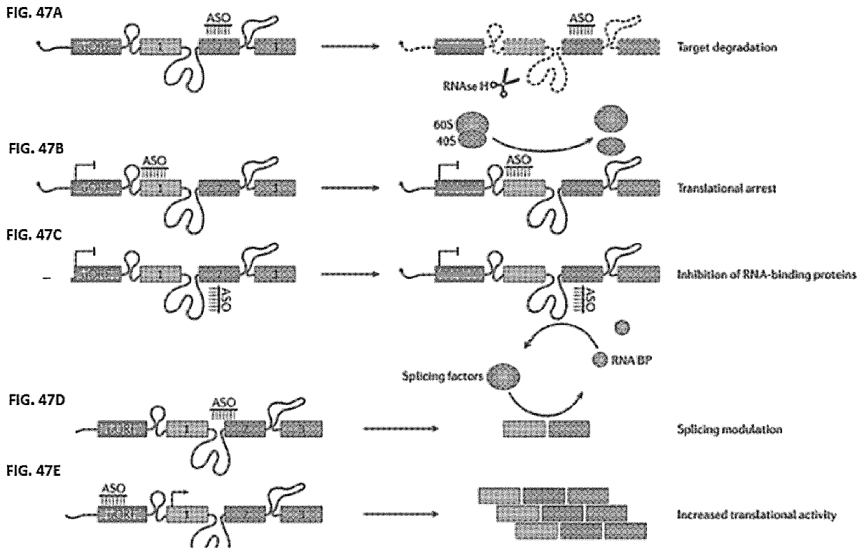

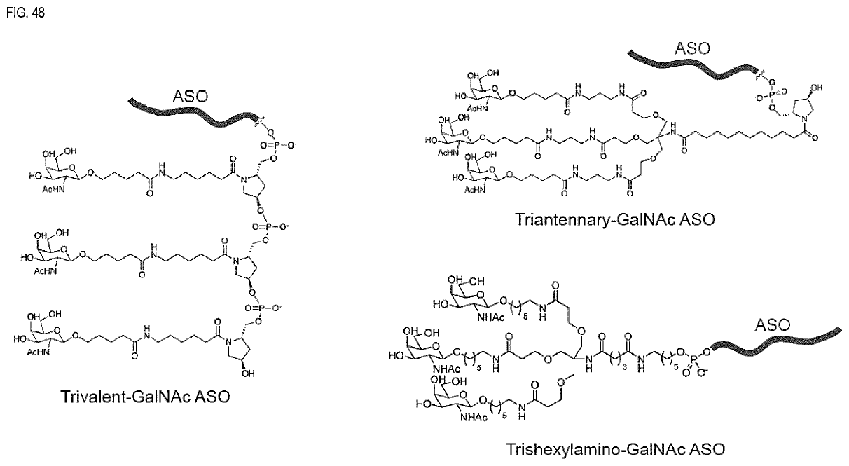

The inventors did not observe any significant difference in weight gain under chow or NASH diet (FIG. 44A) or following a 4-weeks recovery period (FIG. 44B). Similarly, no change was observed in the ratio of liver/body weight or perigonadal adipose tissue weight (FIGS. 17C-F). The only significant difference observed (p-0.0113) was a 5-fold bigger loss of body weight following the recovery period from the HFFC diet in Pcsk7^{-/-} mice compared to WT (FIG. 17G).

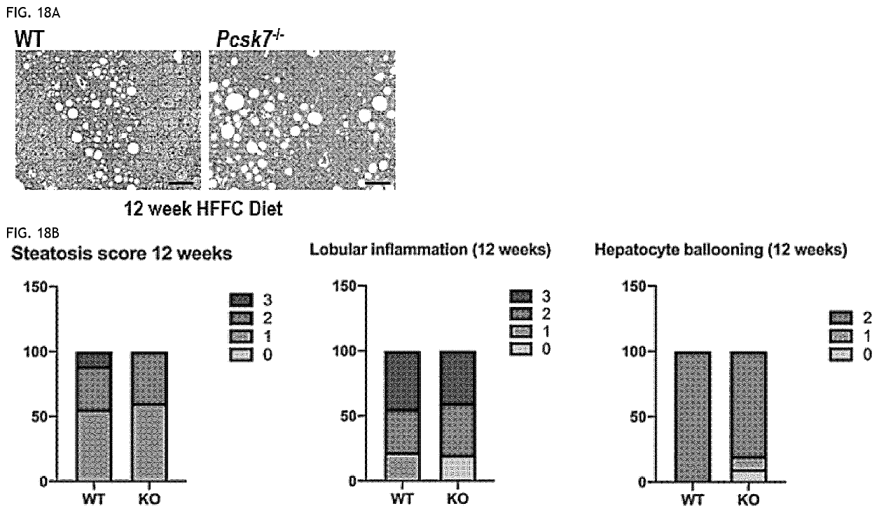

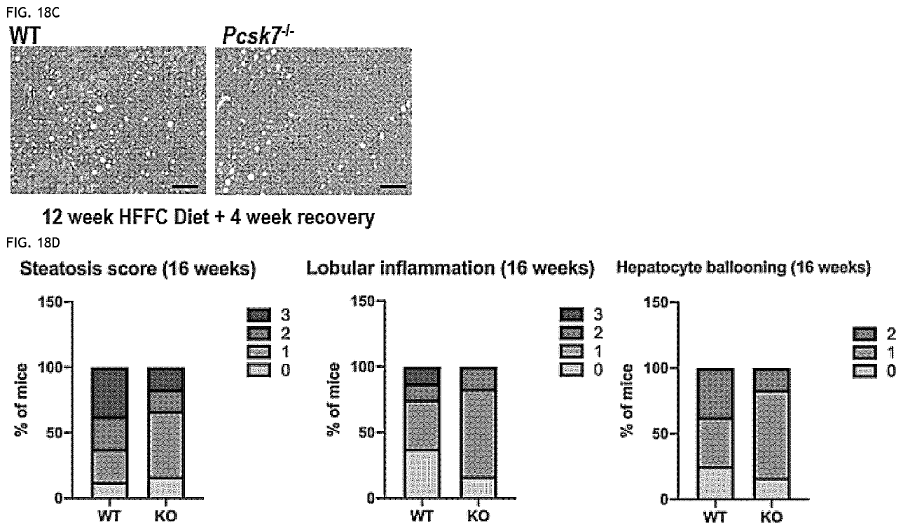

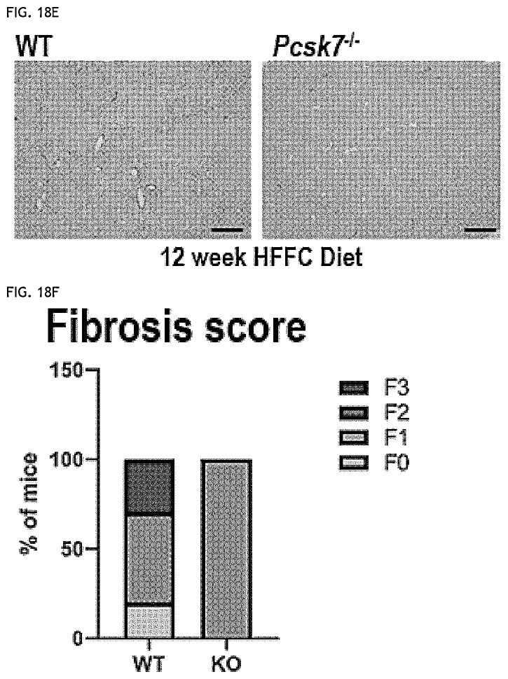

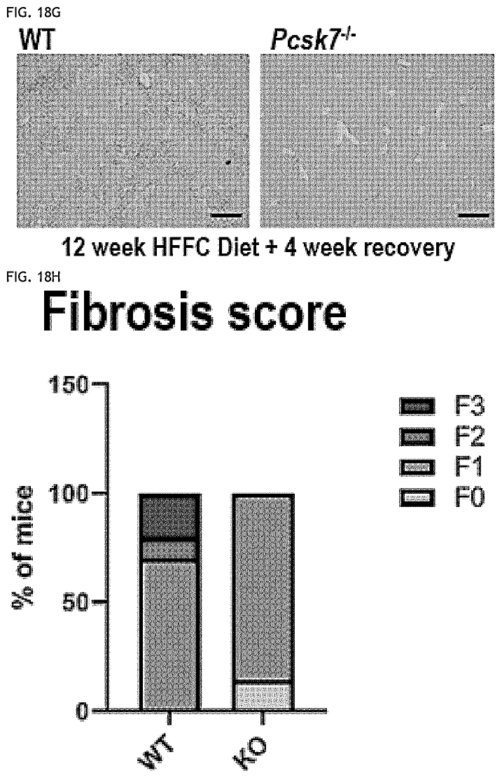

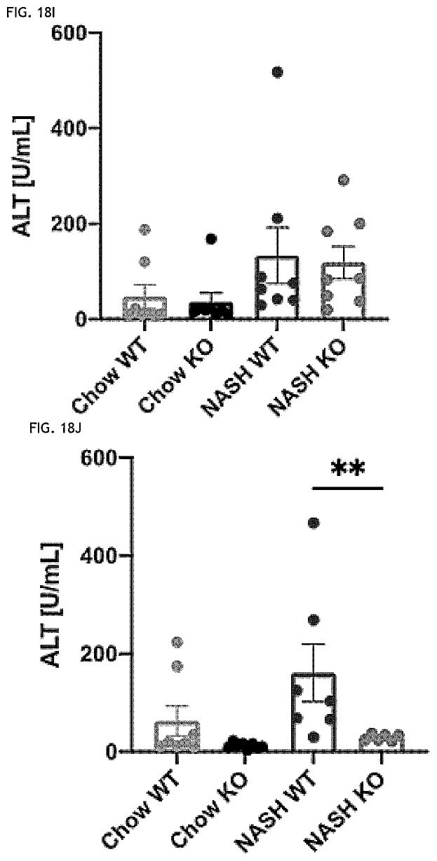

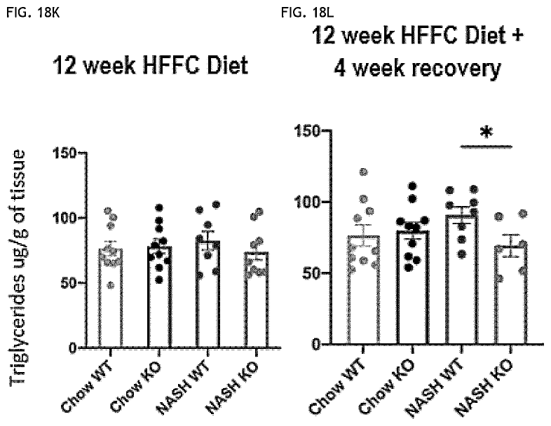

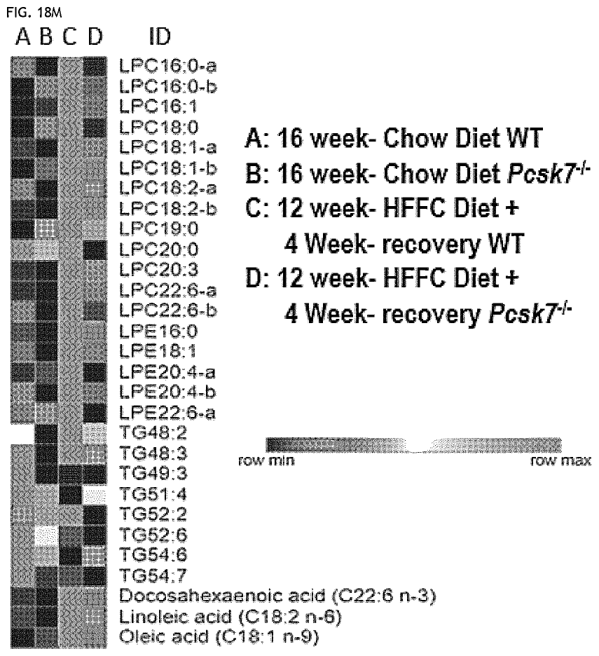

The inventors next examined the pathology developed by the livers of these mice following a HFFC diet, before and after the 4-weeks recovery period (FIGS. 18A-L). After 12 weeks of a HFFC diet the WT and Pcsk7^{-/-} mice exhibited similar grades of steatosis, lobular inflammation, and hepatocyte ballooning (hallmarks of NASH-associated pathology) (FIGS. 18A-B). In contrast, the difference is significantly accentuated following the recovery period, especially for the steatosis score (FIGS. 18C-D) and lobular inflammation, with the Pcsk7^{-/-} mice exhibiting lower grades (FIGS. 18C-D). In addition, an improvement in the fibrosis score was observed in Pcsk7^{-/-} mice at both the pre-recovery and post-recovery periods (FIGS. 18E-H). Moreover, while liver toxicity as assessed by the levels of circulating ALT (Alanine Aminotransferase) is increased in both WT and Pcsk7^{-/-} mice at the pre-recovery period (FIG. 18I), the Pcsk7^{-/-} mice significantly recovered after 4 weeks of normal diet compared to WT (FIG. 18J). Finally, while total liver cholesterol levels do not change in both periods (FIGS. 16E-F), compared to WT the TG levels of Pcsk7^{-/-} mice decreased by ˜25% following the recovery period (FIGS. 18K-L).

The inventors then used an untargeted lipidomic analysis to address the impact of Pcsk7^{-/-} on the hepatic lipidome of mice fed a chow diet and following the recovery period from a NASH diet (Data not shown). The mass spectral (MS) results of chow diet depict the 1168 features obtained following data processing (Data not shown). Using a threshold of P-value <0.05, 30 features discriminated KO from WT mice, of which 12 were annotated using tandem MS analysis (Data not shown). The most significant changes concern the class of triglycerides (TG) for which 9 TG was annotated as being downregulated up to ˜36%. This decrease of several individual TGs is consistent with the inventors' findings supporting lower lipid accumulation in liver through the decreased LD mean area. A similar strategy was then used to compare Pcsk7^{-/-} mice with WT but under a 12-wk HFFC diet followed by a 4-wk recovery period, where 1168 features were obtained (Data not shown). Using a threshold of P-value <0.018, 149 features discriminated Pcsk7^{-/-} from WT mice, of which 31 were annotated using tandem MS analysis (Data not shown). From this, the major significant changes in hepatic lipid classes in Pcsk7^{-/-} compared to WT mice can be summarized as follows: (1) upregulated: 6 glycerophospholipids (from 1.44- to 2.64-fold), 1 TG (1.97-fold), the coenzyme Q9 (1.23-fold), and (2) downregulated: 3 free fatty acids (0.30-fold), 1 TG (0.74-fold) and 19 lysoglycerophospholipids (0.18- to 0.41-fold). To complete this analysis and visualize to which extent the lipids observed as impacted by the HFFC diet are normalized in the Pcsk7^{-/-} mice, a heatmap (FIG. 18M) representing the log 2-transformed averaged signal intensity was performed (https://software.broadinstitute.org/morpheus). Notably, the lipid changes observed under the HFFC diet in a WT background are not observed in Pcsk7^{-/-} mice. Altogether, the latter findings suggest that the loss of Pcsk7 expression prevents the hepatic lipidomics perturbations induced by the HFFC diet.

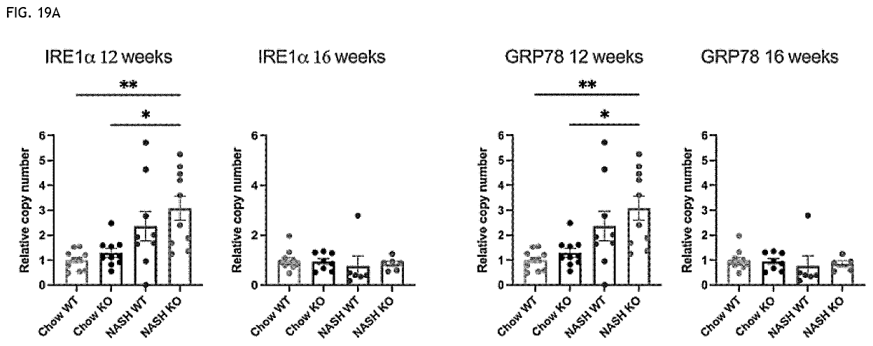

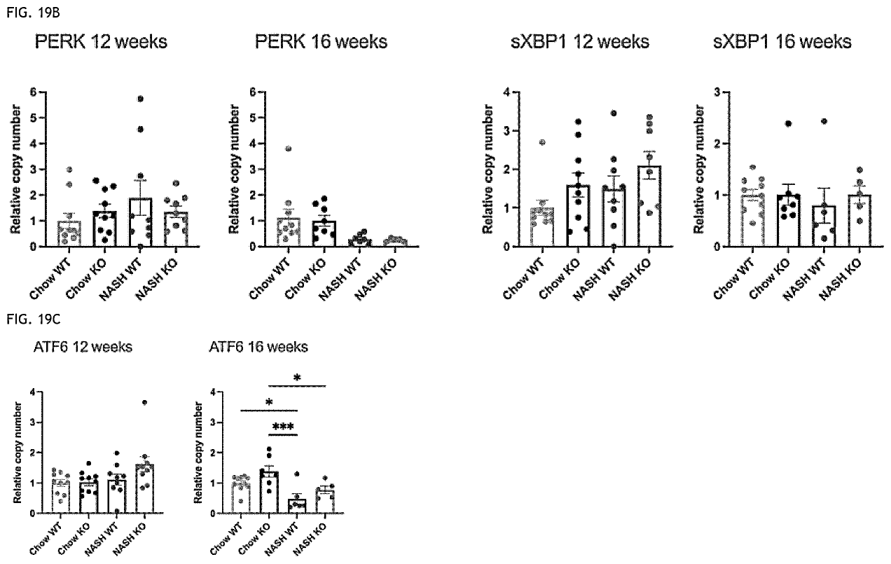

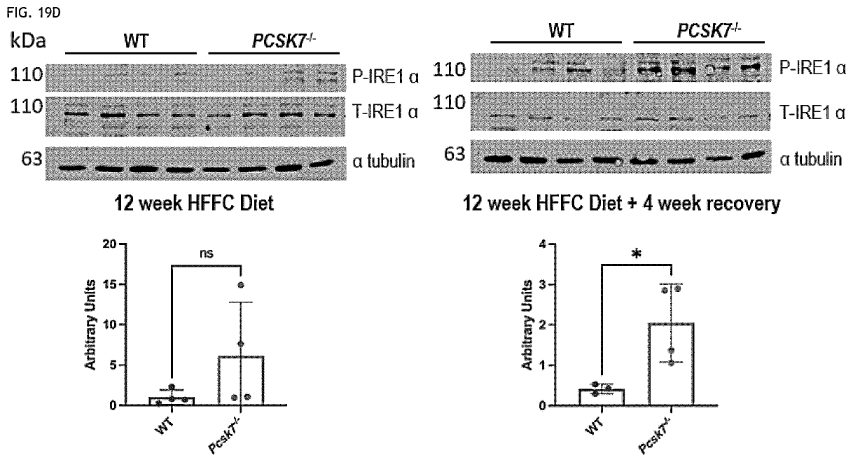

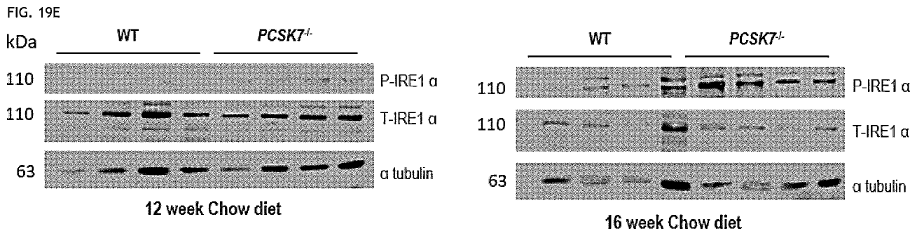

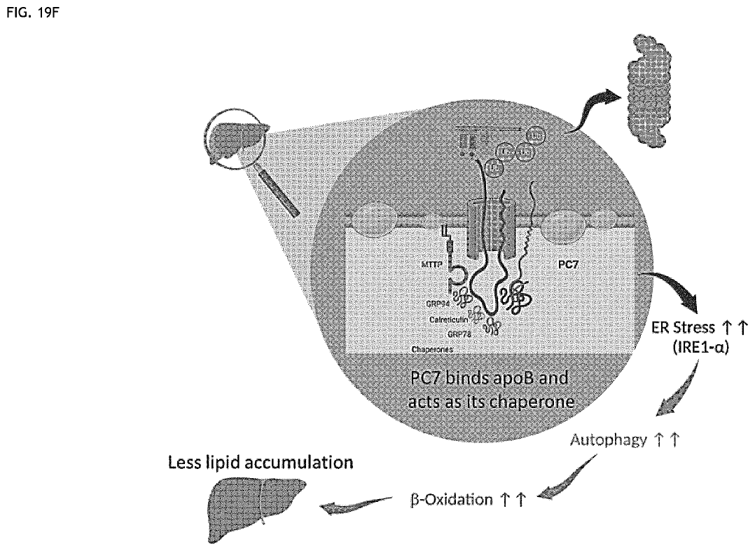

Finally, at the mRNA level a significant increase in the ER-stress marker IRE1a and the ER-chaperone GRP78 was detected only at the 12 weeks period (FIG. 19C), but not for PERK, sXBP1 or ATF6 (FIGS. 19D-E). No mRNA change was observed following the recovery period (16 weeks) for any marker (FIGS. 19C-E). At the protein levels, at both periods an enhanced UPR signaling was observed reflected by higher levels of phoho-IRE1α (P-IRE1α), especially after the recovery period (FIG. 19A). Notably, higher UPR signaling was also observed in mice fed a chow diet for 12 and 16 weeks (FIG. 19F).

In sum, data presented above indicate that PC7 likely acts as an apoB chaperone to allow its efficient exit from the ER. Its absence results in an increased ER-stress leading to enhanced autophagy and lipid β-oxidation, resulting in reduced lipid accumulation in the liver (lower levels of LD), as schematized in the proposed model (FIG. 18B).

### Example 6: Pcsk7^{-/-} Mice do not Show Differences in the Levels of MTP or TM6SF2

The above data revealed that the absence of liver PC7 would lead to lower levels of apoB in liver and in circulation, as observed in Pcsk7^{-/-} mice, without affecting the protein levels of microsomal triglyceride transfer protein (MTP) (PMID: 22121032) or transmembrane 6 superfamily member 2 (TM6SF2) (PMID: 34923175) (FIGS. 20A-B), both recognized as major apoB chaperones. In sum, data presented herein suggest that PC7 could act as a novel molecular chaperone to apoB, enhancing its exit from the ER and secretion from hepatocytes.

### Example 7: Human Gain-of-Function (GOF) of PC7

Human genetic evidence from large-scale biobanks supports the association of PC7 levels with circulating lipids and apolipoproteins (FIG. 21A). The rs236918 variant is a polymorphism affecting PC7 activity (PMID: 21149283) and levels (PMID: 30918065). On average, for every additional GOF PCSK7 C-allele, carriers have 0.09% higher TG levels (p=4.6×10⁻¹¹⁹), 0.04 mg/dl higher total cholesterol (TC) levels (p=1.9×10⁻²⁴), 0.54 mg/dl higher apoB levels (p=8.1×10⁻¹³) and 0.56 mg/dl higher apolipoprotein A1 levels of (p=2.5×10⁻¹⁰). In contrast, carriers exhibit 0.02 mg/dl lower high density lipoprotein cholesterol (HDLc) (p=2.7×10⁻⁵) (FIG. 21A). These data confirm the association of PCSK7 GOF with enhanced cardiometabolic risk factors such as TG and apoB, resulting in a relatively minor change in their levels.

### Example 8: Human Loss-of-Function (LOF) of PC7

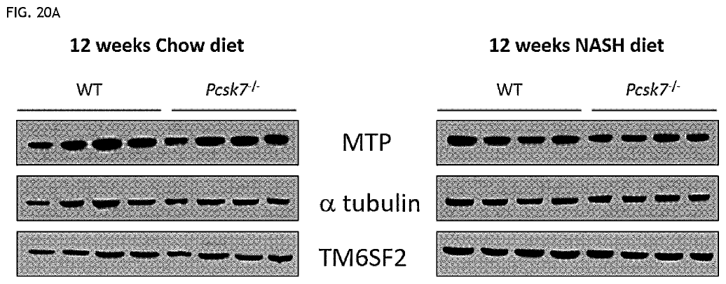

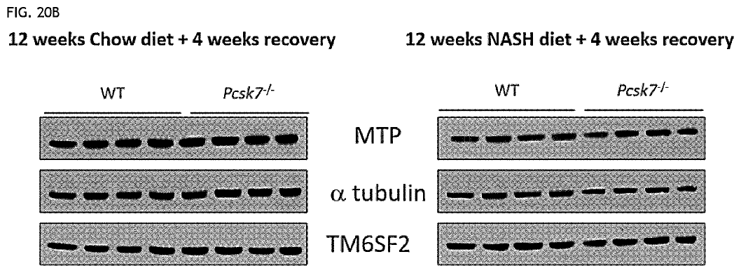

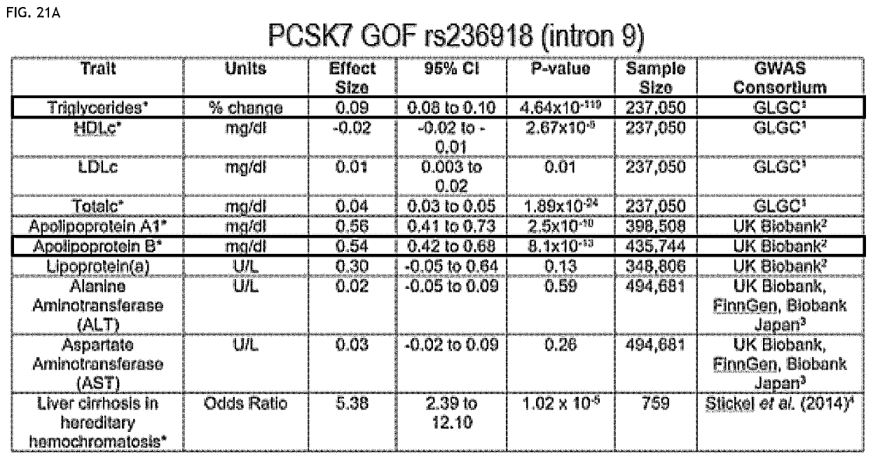

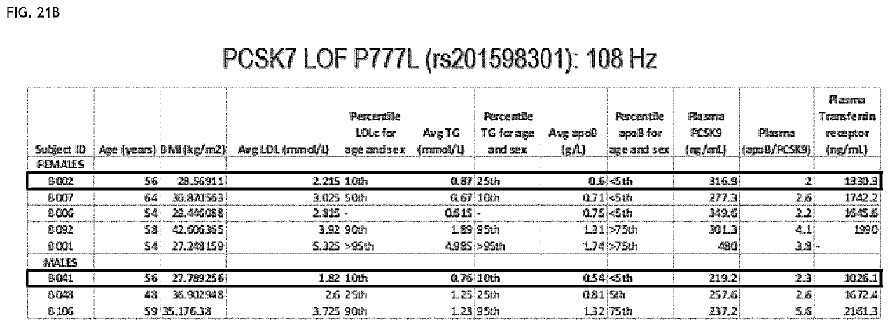

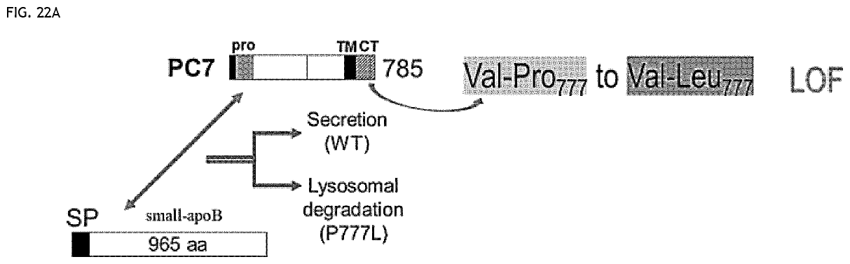

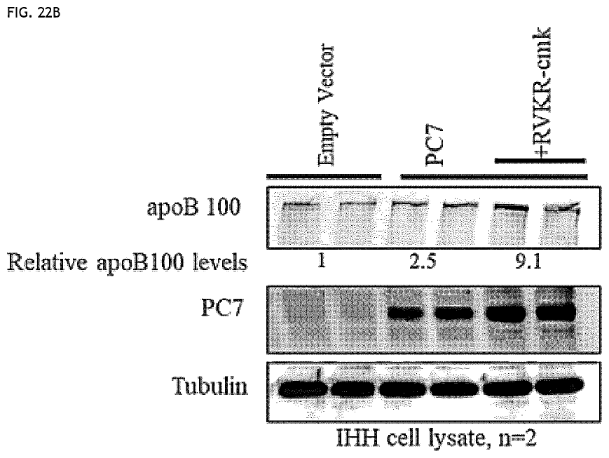

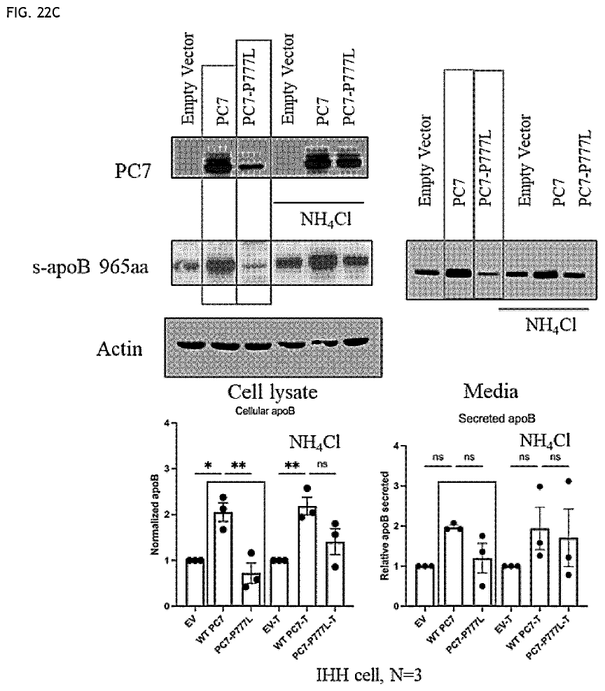

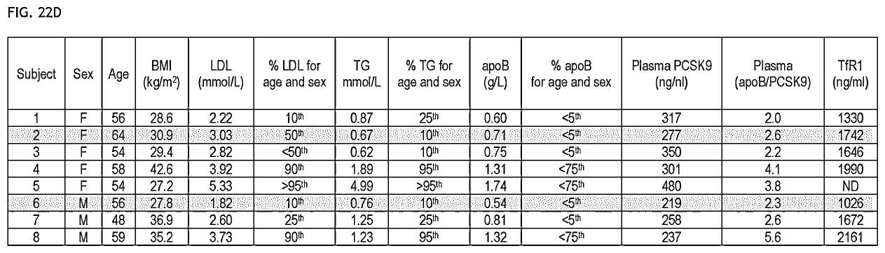

Possible human PCSK7 variants that may be associated with loss-of-function of PC7 and hence leading to lower TG levels were then searched. Accordingly, individuals who exhibited low ApoB and TG levels were selected (collaboration Dr May Faraj, IRCM). This led to the identification of a man (B041) and a woman (B007) who have low TG and ApoB in the 5^{th} percentile (FIG. 21B). In the case of the man (B041) this phenotype is associated with low plasma LDL-cholesterol in the 10th percentile (FIG. 21B). DNA sequencing revealed that both exhibit a heterozygote P777L variant of PCSK7. This modification occurs in the cytosolic tail of PC7 and could potentially regulate its subcellular trafficking and localization, as it transforms a Val-Pro₇₇₇ sequence into di-aliphatic Val-Leu₇₇₇ (Durand et al., 2020). It is speculated herein that trafficking of [WT PC7-ApoB] vs [PC7-P777L-ApoB]complex is affected and that the latter is probably more efficiently directed to lysosomes for degradation of ApoB (FIGS. 22A-C), thereby providing a rationale for the observed low circulating levels of ApoB (FIG. 21B).

In cell-based assays this variant resulted in a 50% reduction in the ability of PC7 to shed its only known specific membrane-bound substrate transferrin receptor 1 (hTfR1) (Guillemot et al., 2013; Durand et al., 2020), revealing the first validated loss-of-function (LOF) variant of PC7, also likely due to lower PC7 concentration in early endosomes where cleavage of hTfR1 occurs (FIGS. 23A-B) (Durand et al., 2020). Amazingly, FPLC analysis of the plasma of man subject B041 revealed low concentrations of both LDL-cholesterol and VLDL-TG, but no change in HDL-cholesterol or HDL-TG compared to a control man (RE) with similar weight (FIGS. 24A-B). Thus, in men it is possible that LOF of PC7 could result in low ApoB, TG and LDL-cholesterol. Database analysis from the Genome Aggregation Database (gnomAD v2.1.1 and v3.1.2 combined, gnomAD from broadinstitute: P777L; RS201598301) revealed 8,115 Heterozygote & 175 Homozygote P777L being reported (see Tables 1 and 2 below). Thus, homozygote P777L LOF is not apparently associated with morbidity, as was also observed with Pcsk7 KO mice.

TABLE 1
Population frequencies of P777L including exomes and genomes

| | Allele | Allele | Number of | Allele |
|---|---|---|---|---|
| Population | Count | Number | Homozygotes | Frequency |
| South Asian | 1176 | 14230 | 59 | 0.08264 |
| East Asian | 488 | 13490 | 10 | 0.03617 |
| European (non-Finnish) | 1998 | 73302 | 28 | 0.02726 |
| Ashkenazi Jewish | 123 | 4516 | 0 | 0.02724 |
| Other | 118 | 4602 | 4 | 0.02564 |
| European (Finnish) | 287 | 17396 | 5 | 0.01650 |
| Latino | 292 | 20078 | 1 | 0.01454 |
| African | 91 | 17340 | 1 | 0.005248 |
| Male | 2619 | 87328 | 63 | 0.02999 |
| Female | 1954 | 77626 | 45 | 0.02517 |
| Total | 4573 | 164954 | 108 | 0.02772 |

Include: ✓ Exomes ✓ Genomes
Genome Aggregation Database (gnomAD) from Broad institute.

TABLE 2
Population frequencies of P777L including genomes

| | Allele | Allele | Number of | Allele |
|---|---|---|---|---|
| Population | Count | Number | Homozygotes | Frequency ^{▾} |
| South Asian | 253 | 2914 | 15 | 0.08682 |
| East Asian | 113 | 3110 | 3 | 0.03633 |
| European (non- | 2043 | 64286 | 29 | 0.03178 |
| Finnish) | | | | |
| Other | 43 | 2126 | 1 | 0.02023 |
| Ashkenazi Jewish | 61 | 3312 | 2 | 0.01842 |
| Latino | 225 | 13546 | 4 | 0.01661 |
| European (Finnish) | 164 | 10380 | 1 | 0.01580 |
| African | 256 | 41240 | 2 | 0.006208 |
| Amish | 5 | 898 | 0 | 0.005568 |
| Male | 1607 | 68662 | 45 | 0.02340 |
| Female | 1556 | 73150 | 12 | 0.02127 |
| Total | 3163 | 141812 | 57 | 0.02230 |

Include: Exomes Genomes
Genome Aggregation Database (gnomAD) from Broad institute.

### Example 9: Material and Method for Testing miRNA Effect on PCSK7 Expression and Function

Computational Prediction of miRNAs that Target the PCSK7 Gene

The following publicly available bioinformatics algorithms were employed to predict potential miRNAs that target the PCSK7 gene: TargetScan (http://www.targetscan.org/vert_71/), DIANA tools (http://diana.imis.athena-innovation.gr/DianaTools/index.php), miRDB (http://mirdb.org/), miRanda (http://www.microrna.org/microrna/home.do), and the UCSC website (http://genome.ucsc.edu).

### Cell Culture

Human embryonic kidney 293 (HEK293T), hepatocellular carcinoma (HepG2), and hepatoblastoma (Huh7) cells were selected for further functional experiments. The HEK293T and Huh7 cell lines were cultured in Gibco Dulbecco's Modified Eagle Medium (DMEM) (Invitrogen, USA), supplemented with 100 U/mL of penicillin, 100 μg/mL of streptomycin (Sigma, USA), and 10% fetal bovine serum (FBS) (Invitrogen, USA); then, they were incubated at 37° C. with 5% CO₂. The HepG2 cells were cultivated in DMEM-F12 (Invitrogen, USA), containing 10% FBS, 100 U/mL of penicillin, and 100 μg/mL of streptomycin.

RNA Extraction, Complementary DNA (cDNA) Synthesis, and Quantitative Polymerase Chain Reaction (qPCR)

RNA was isolated from the cells with the TRIzol™ Reagent (Invitrogen, USA) according to the manufacturer's instructions. The RNA samples were treated with RNase-free DNase I (Fermentas, USA) to eliminate any possible DNA contamination. Reverse transcription was performed using the PrimeScript™ First Strand cDNA Synthesis Kit (Takara, Japan) following the manufacturer's protocol. Briefly, one unit of the DNase I enzyme, 1 μL of a buffer, and 1 μg of total RNA were incubated for 30 minutes at 37° C. Then, 1 μL of 50 mM EDTA was added for enzyme inactivation and incubated at 65° C. for 10 minutes. Subsequently, 5 μL of DNase-treated RNA was added to the mix of 0.5 μL of the reverse-transcriptase enzyme, 2 μL of the reverse-transcriptase buffer, and 1 μL of a random hexamer and incubated for 15 minutes at 37° C., followed by 5 seconds at 85° C. for enzyme inactivation. Stem-loop RT-qPCR method was applied for evaluating the expression of miRNAs (Kramer, 2011).

The qPCR test was conducted in 20 μL of the PCR reaction mixture using SYBR Green I (Takara, Japan) in an Applied Biosystems StepOne™ instrument (Applied Biosystems, USA). Briefly, cDNA equivalent to 50 ng of RNA was added to the mix of 10 μL of SYBR Green, 0.5 μM of each primer, 0.4 μL of the ROX reference dye, and sufficient water. The real-time thermal program was as follows: 95° C. for 30 seconds, 40 cycles at 95° C. for 20 seconds, and 60° C. for 35 seconds for PCSK7, as well as 95° C. for 30 seconds, 40 cycles at 95° C. for 5 seconds, 60° C. for 30 seconds, and 72° C. for 15 seconds for miR-125a-5p, miR-143-3p, miR-409-3p, miR-320a-3p, and miR-244, for which RNU48 small nuclear RNA, β2-microglobulin (β2m), and GAPDH mRNAs were used as internal controls.

All the reactions were repeated in duplicates. Next, melt curves were analyzed, with the mean threshold cycles used for further analyses. The relative expressions of the miRNAs and PCSK7 to RNU48 small nuclear RNA and/or GAPDH and β2m were calculated, respectively, via the 2-ΔΔCt method. The PCR products were sequenced (3500 ABI) to validate the accuracy of the amplification. All the primers for PCSK7 and the nominated miRNAs are listed in Table 3 below.

TABLE 3
primers for PCSK7 and miRNAs

| | | |
|---|---|---|
| | Forward/ | |
| Names | Reverse | Sequences |

Expression primers

| | | |
|---|---|---|
| miR-125 | F | ACGCATCCCTGAGACCCTT (SEQ ID NO: 43) |
| miR-125 | R | CAGTGCAGGGTCCGAGGTA (SEQ ID NO: 44) |
| Stem-loop | | GTCGTATCCAGTGCAGGGTCCGAGGTATTCGCACTGGATACGACTCACAG |
| | | (SEQ ID NO: 45) |
| miR-143 | F | GCACGCATGAGATGAAGCAC (SEQ ID NO: 46) |
| miR-143 | R | AGTGCAGGGTCCGAGGTA (SEQ ID NO: 47) |
| Stem-loop | | GTCGTATCCAGTGCAGGGTCCGAGGTATTCGCACTGGATACGACGAGCTA |
| | | (SEQ ID NO: 48) |
| miR-320 | F | ACGCAAAAAGCTGGGTTGAG (SEQ ID NO: 49) |
| miR-320 | R | AGTGCAGGGTCCGAGGTA (SEQ ID NO: 50) |
| Stem loop | | GTCGTATCCAGTGCAGGGTCCGAGGTATTCGCACTGGATACGACTCGCCC |
| | | (SEQ ID NO: 51) |
| miR-409-3p | F | GGGAATGTTGCTCGGTG (SEQ ID NO: 52) |
| miR-409-3p | R | CAGTGCAGGGTCCGAGGTA (SEQ ID NO: 53) |
| Stem loop | | GTCGTATCCAGTGCAGGGTCCGAGGTATTCGCACTGGAT (SEQ ID NO: 54) |
| RNU48 | F | TGATGACCCCAGGTAACTCTG (SEQ ID NO: 55) |
| RNU48 | R | AGTGCAGGGTCCGAGGTA (SEQ ID NO: 56) |
| Stem-loop | | GTCGTATCCAGTGCAGGGTCCGAGGTATTCGCACTGGATACGACGGTCAG |
| | | (SEQ ID NO: 57) |
| PCSK7 | F | ACACCATCCAGGACATTGCA (SEQ ID NO: 58) |
| PCSK7 | R | GGGTCATTAGAGTTGAGGTCATAGC (SEQ ID NO: 59) |
| B2m | F | GGGTTTCATCCATCCGACATTG (SEQ ID NO: 60) |
| B2m | R | TGGTTCACACGGCAGGCATAC (SEQ ID NO: 61) |
| GAPDH | F | ATGGGGAAGGTGAAGGTCG (SEQ ID NO: 62) |
| GAPDH | R | GGGGTCATTGATGGCAACCAATA (SEQ ID NO: 63) |

Cloning primers

| | | |
|---|---|---|
| PCSK7-3′UTR | F | CCCTCGAGCCTCAGGGCCTGACAGTGT (SEQ ID NO: 64) |
| PCSK7-3′UTR | R | TTGCGGCCGCATTGTTTGGAAGGTTTTTATTTTGG (SEQ ID NO: 65) |
| miR-125 | F | GGTCTTTCTGTCTCTGGC (SEQ ID NO: 66) |
| miR-125 | R | AGGTTTCAGTTGGTGGTC (SEQ ID NO: 67) |
| miR-143 | F | CGGAATTCCTAACACCCCTTCTCCTGGC (SEQ ID NO: 68) |
| miR-143 | R | CGGGATCCTTCCCCAGCATCACAAGTGG (SEQ ID NO: 69) |
| miR-409 | F | GTCTTCTGCAAGCACAGCC (SEQ ID NO: 70) |
| miR-409 | R | CAATTACTTTCCAACTGGTCGAC (SEQ ID NO: 71) |
| miR-320a | F | CGGAATTCGGGGCGGAAGTGACGTTAG (SEQ ID NO: 72) |
| miR-320a | R | CGGGATCCACCCTGATCTTGGCGCCT (SEQ ID NO: 73) |

SOEing primers

| | | |
|---|---|---|
| 125-1-mut a F | F | CCCTCGAGTGGGACAGGCTCTTCTTT (SEQ ID NO: 74) |
| 125-2-mut b1 | R | AGATAGCTGGCCTCCGGCGGAAGACAGGGTGGTGGCAG (SEQ ID NO: 75) |
| 125-2-mut b2 | F | CTGCCACCACCCTGTCTTCCGCCGGAGGCCAGCTATCT (SEQ ID NO: 76) |
| 143-1-mut a | R | CTAGGCAGCTGGCTTGGCAGGCCAAACCAAAGGGGGG (SEQ ID NO: 77) |
| 143-1-mut a | F2 | CCCCCCTTTGGTTTGGCCTGCCAAGCCAGCTGCCTAG (SEQ ID NO: 78) |
| 143-2-mut b | F2 | GTTGAGAAGGTGCCTGCCAAAACATCACTGGGGTCACTT (SEQ ID NO: 79) |
| 143-2-mut b | R1 | AAGTGACCCCAGTGATGTTTTGGCAGGCACCTTCTCAAC (SEQ ID NO: 80) |
| 143-mut | R1 | AAGTGACCCCAGTGATGTTTAGGCCAAACCAAAGGGGGG (SEQ ID NO: 81) |
| 143 mut | F2 | CCCCCCTTTGGTTTGGCCTAAACATCACTGGGGTCACTT (SEQ ID NO: 82) |
| mut 409 | R | TTGCGGCCGCACCAACTAGCTCAGAAGAGGG (SEQ ID NO: 83) |

### Plasmids and Cell Transfection MiR-Overexpressing Vectors

The effects of the selected miRNAs on the mRNA expression level of PCSK7 were examined 48 hours after the transfection of the overexpressing miRNAs in the Huh7 and HEK293T cell lines. Plasmids encoding pEGFP-C-miR-125a-5p, miR-143-3p, miR-409-3p, and miR-320a-3p and their corresponding control (miR-NC) were constructed. The nominated miRNA genes were amplified and cloned downstream of the GFP gene into the pEGFP-C1 vector (Clontech, Japan). All the primer sequences used are available in the above Table 3 above.

Pre-miR miRNA precursor-overexpressing vectors (300 ng) were transfected in the Huh7 and HEK293T cell lines using FuGENE™ HD (Promega Corporation, Madison, WI, USA) in 12-well plates. The transfections were carried out in triplicate, and mock-related counterpart vectors were utilized as controls.

### Vectors Containing the PCSK7 3′-UTR Wild Type and Mutated Forms

The interactions between the miRNAs and their probable targets on PCSK7 were explored by cloning the potential target regions in psiCHECK-2™ (Promega, USA), a luciferase reporter vector. In psiCHECK-2™, hRluc, the Renilla luciferase gene, is located upstream of the target regions of interest cloned into the psiCHECK-2™ vector downstream of the Renilla gene. The region corresponding to the 3′-UTR of PCSK7 (926-nt sequences in length) that constituted the predicted miRNA response elements was PCR-amplified and cloned downstream of the Renilla luciferase gene in the psiCHECK-2™ vector (Promega, USA). For the confirmation of whether miRNA response elements (miRNA target sites) on the 3′-UTR of PCSK7 were active and had direct interactions with the miRNAs, different mutants (plasmids) were constructed via splicing by over-hang-extension (SOEing) PCR. For miR-125a-5p and miR-143-3p, each of which has two miRNA response elements on PCSK7 3′-UTR, three different mutant constructs were built. Two constructs were made by deleting a putative miRNA target site, and the third one was made by omitting both putative miRNA target sites in the 3′-UTR sequence of PCSK7. In the miR-409-3p mutant construct, a putative miRNA target site was deleted from the 3′-UTR sequence. The sequences of the primers are listed in Table 3 above.

### Luciferase Reporter Assay

The HEK293T cells were co-transfected through the application of the wild-type psiCHECK-2, the wild-type PCSK7 3′-UTR, the mutant PCSK7 3′-UTR, and the miR-overexpressing vectors so that the direct interactions of the nominated miRNAs with PCSK7 3′-UTR could be investigated. In brief, 150 ng of the wild-type or mutated 3′-UTR constructs and 300 ng of the miRNA-expressing vectors were co-transfected in HEK293T-cultured 48-well plates using FuGENE™ (Invitrogen, USA). Additionally, the psiCHECK-2 and pEGFP-C1 mock vectors were transfected and utilized as controls for luciferase assay and transfection, respectively. Transfection efficiency was monitored by fluorescent microscopy (Nikon TE2000S, Japan) 36 hours following the procedure.

The PsiCHECK-2 reporter construct plasmid contained the Renilla luciferase gene upstream of PCSK7 3′-UTR and an independent firefly luciferase gene as an internal control for normalization. Forty-eight hours after HEK293T co-transfection, the luciferase reporter assay was carried out employing the Dual-Luciferase Reporter Assay System (Promega, USA) with a luminometer (Titertek-Berthold, Germany) in accordance with the manufacturer's protocol. Each sample was performed in triplicate, and the experiment was repeated at least three biological times. In short, a lysis buffer was added to each well after the removal of the media of the cell. Then, LARII Reagent was added, and after 20 minutes, the firefly luciferase activity was measured as a control. Afterward, Renilla activity was determined using Stop & Glo Reagent. The relative luciferase activity was calculated using the following formula: ΔFold Activity of Luciferase (Renilla/Firefly)=Average Renilla/Firefly from Samples A/B.

### Western Blot Analysis

In the next step, the effect of miR-125a-5p on PC7 function was determined. First, the miR-125a-5p-overexpressing vector and its related mock plasmids were co-transfected with the pIRES vector (Invitrogen, USA), containing the full length of cDNA encoding the PCSK7 mRNA with the complete 3′-UTR in Huh7 cells. The western blot analysis was performed 48 hours after transfection. Thereafter, the impact of miR-125a-5p on the enzymatic function of PC7 was assessed by co-transfecting Huh7 cells with the miR-125a-5p-overexpressing vector, with that coding for the full length of PCSK7, and a plasmid encoding hTfR1 (Guillemot et al., 2013; Durand et al., 2020). Cell lysates and media were collected for the western blot analysis 48 hours after transfection.

Subsequently, proteins were isolated with an ice-cold RIPA buffer (1×), comprising 50 mM of Tris hydrochloride (pH 8), 150 mM of sodium chloride, 0.1% sodium dodecyl sulfate, 1% Nonidet P40, 0.25% sodium deoxycholate, and a cocktail of protease inhibitors (Roche, Oakville, ON, Canada). The proteins were subjected to electrophoresis on 12% of polyacrylamide sodium dodecyl sulfate gels and blotted to polyvinylidene fluoride (PVDF) membranes. The PVDF membranes were subsequently blocked by fat-free 5% milk powder dissolved in Tris-buffered saline (0.1 M of Tris hydrochloride [pH 8] and 1.5 M of sodium chloride), containing 0.1% Tween-100 (TBS-T). Both PC7 and TfR1 were C-terminally tagged with V5 and detected with a V5-monoclonal antibody (Invitrogen) and membranes were incubated with appropriate primary and secondary antibodies, as reported (Guillemot et al., 2013). Subsequently, immunoreactive bands (the signal) were visualized with an enhanced chemiluminescent reaction kit (Bio-Rad, USA) and recorded via chemiluminescence. The bands were analyzed and quantified using the NIH ImageJ software (US National Institutes of Health, Bethesda, MD, USA).

### Statistical Analysis

The 2-(ΔΔCt) method was applied for the qPCR data analysis and gene expression determination. GraphPad™ Prism, version 8, (GraphPad Software, Inc, La Jolla, CA, USA) was employed to analyze the data obtained via the qPCR, dual-luciferase, and western blot analyses, as well as P-value calculation. A P value less than 0.05 was considered statistically significant for all the experiments.

### Example 10: Bioinformatics Prediction of PCSK7-Targeting miRNAs

The molecular mechanisms underlying PCSK7 expression regulation by miRNAs were investigated via a bioinformatics analysis of five different publicly available target prediction programs, namely TargetScan, DIANA-micro-T, miRDB, miRanda, and UCSC, to predict miRNAs targeting the 3′-UTR of the PCSK7 transcript. Several miRNAs were identified by these programs, leading to the selection for further analysis of four different targeting miRNAs commonly predicted by all programs, namely miR-125a-5p, miR-143-3p, miR-409-3P, and miR-320a-3p. Among the chosen miRNAs, miR-125a-5p had four target sites, two of which are in the 3′-UTR and the rest are situated on exons 14 and 15 of PCSK7 and miR-143-3p has two target sites within the 3′-UTR of PCSK7. In contrast, miR-409-3P and miR-320a-3p have only one predicted target site each (FIG. 25A).

### Example 11: Expression of the Predicted miRNAs and PCSK7 in HEK293T, HepG2, and Huh7 Cell Lines

The next stage was an evaluation of the endogenous expression patterns by qPCR of the four selected miRNAs (miR-125a-5p, miR-143-3p, miR-409-3p, and miR-320a-3p) and PCSK7 mRNA in three cell lines, namely kidney-derived HEK293T, hepatocytes-derived HepG2, and Huh7 cell lines (FIG. 25B).

Huh7 cells showed the highest relative expression of PCSK7 mRNA, very low RNA expression levels of miR-143-3p and miR-409-3p, whereas the expression levels of miR-125a-5p and miR-320a-3p were similar in all cell lines. In HepG2 and HEK293T cells, the expression levels of miR-125a-5p and miR-320-3p were comparable to that of PCSK7, while the expression levels of miR-143-3p and miR-409-3p were low. From these data, it seems that the levels of PCSK7 mRNA is inversely correlated to the RNA levels of miR-143-3p and miR-409-3p in all cells, whereas miR-125a-5p may negatively regulate PCSK7 mRNA levels in Huh7 cells.

### Example 12: Negative Regulation of PCSK7 mRNA Expression Levels by the Overexpression of miR-125a-5p, miR-143-3p, and miR-409-3p

Huh7 cells were transfected with miRNA-overexpressing vectors carrying the precursors of miRNA sequences and mock-related counterpart vectors to assess the possible relationship between the expression patterns of miR-125a-5p, miR-143-3p, miR-409-3p, and miR-320a-3p and PCSK7 expression at the transcriptional level. The qPCR results obtained at 48 hours following transfection demonstrated that the overexpression of miR-143-3p and miR-409-3p in Huh7 cells significantly decreased by 40-50% the expression of PCSK7 mRNA compared to cells transfected with the mock vector (P=0.0158 and P=0.0084) (FIG. 26A). Although overexpression of miR-125a-5p tended to reduce the expression of PCSK7 mRNA in these cells, this decrease did not reach statistical significance (P=0.0693). In contrast, the ectopic expression of miR-320a-3p did not downregulate the expression of PCSK7 in Huh7 cells (P=0.5538).

The association between the expression levels of miR-125a-5p and miR-320a-3p and the expression level of PCSK7 was further investigated by measuring the levels of PCSK7 mRNAs in HEK293T cells. The qPCR results showed that the overexpression of miR-125a-5p now led to a significant ˜25% reduction in PCSK7 mRNA levels 48 hours after transfection in these cells (P=0.0114) (FIG. 26B). However, like Huh7 cells, no significant regulatory relationship was observed for miR-320a-3p in HEK293T cells (P=0.9972). Overexpression of miR-143-3p, miR-409-3p and less so miR-125a-5p may thus downregulate PCSK7 expression.

### Example 13: Direct Interaction Between the Predicted miRNAs and the 3′-UTR of PCSK7

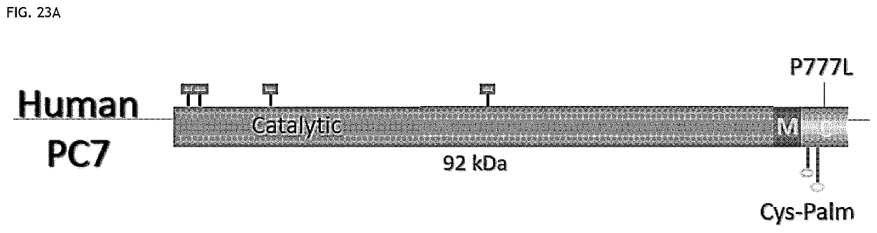

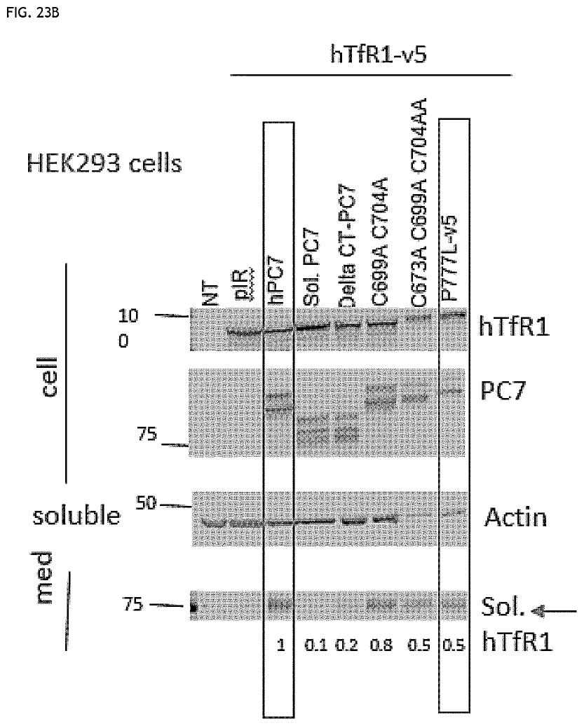

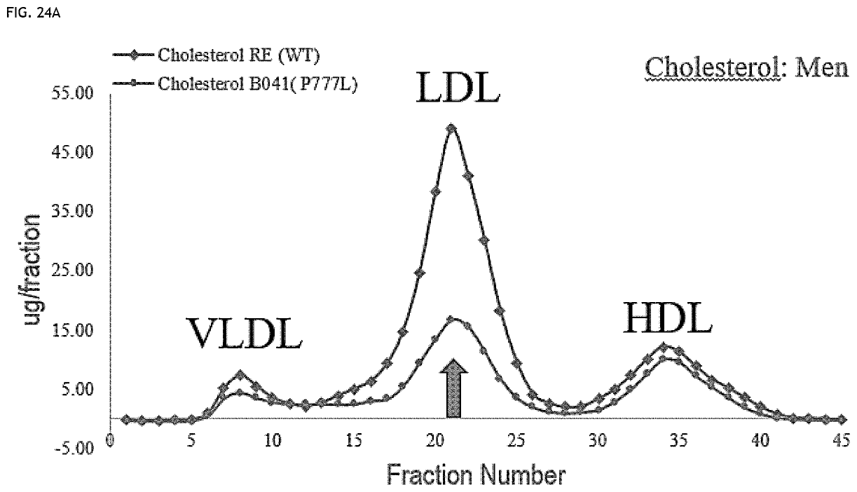

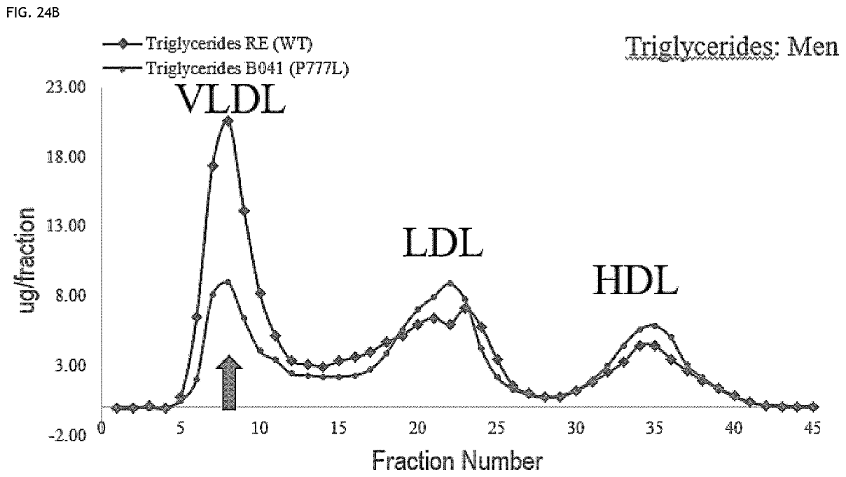

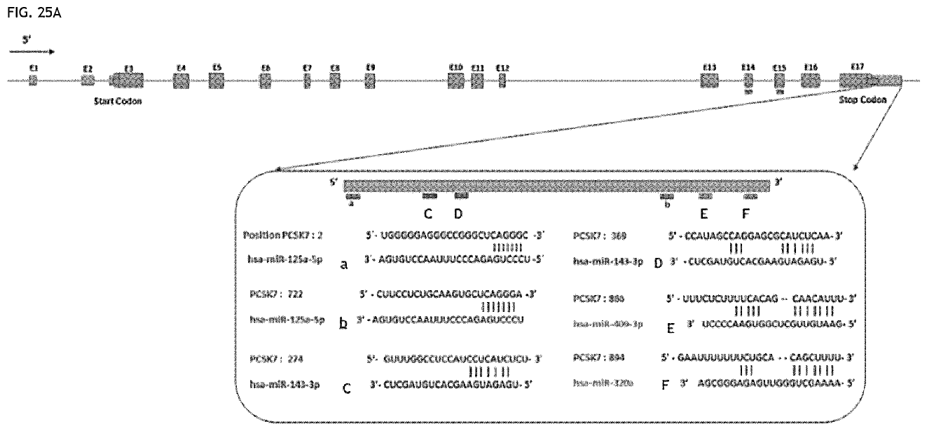

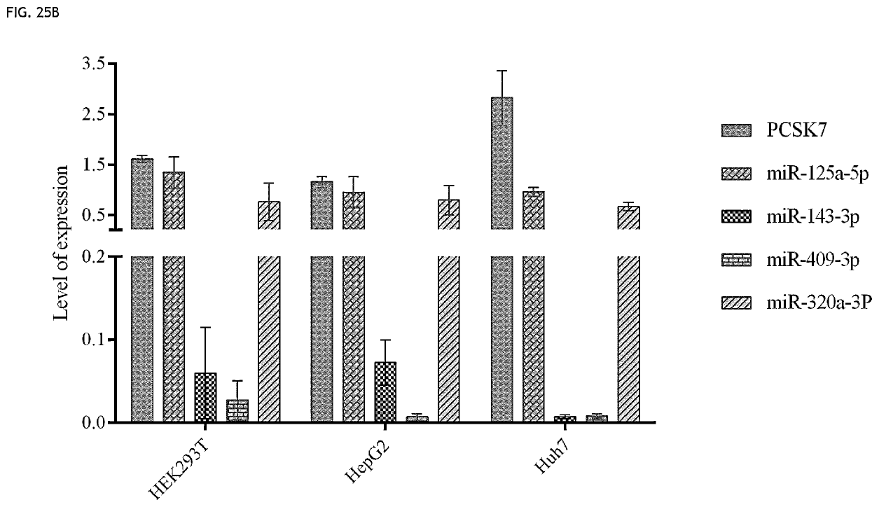

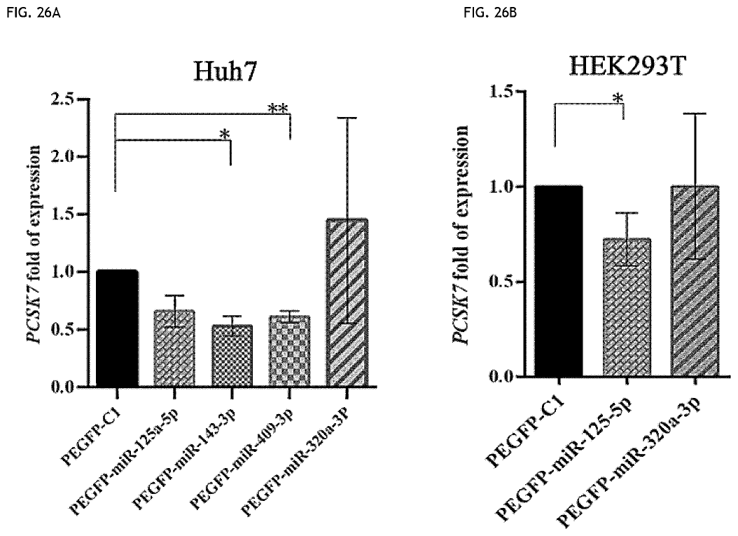

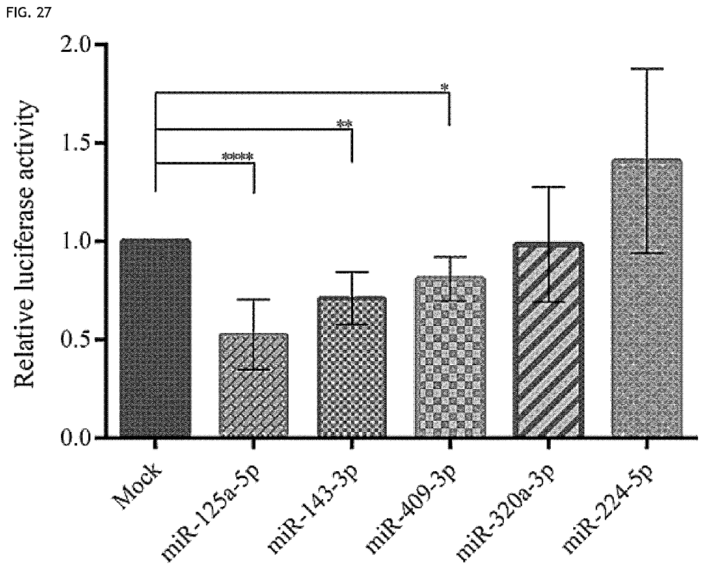

A dual-luciferase assay was applied to investigate the interaction between the predicted miRNAs and the 3′-UTR of human PCSK7 mRNAs, cloned downstream of the Renilla luciferase gene in the psiCHEK-2 plasmid. Additionally, miR-224-5p without any target site on the 3′-UTR of PCSK7 served as control. Overexpression of miR-125a-5p significantly reduced by ˜50% the relative luciferase activity by ˜50% (P<0.0001) in the HEK293T cells co-transfected with the miR-125a-5p-overexpressing plasmid and a psiCHECK-2 carrying the wild-type PCSK7 3′-UTR (FIG. 27). Likewise, miR-143-3p and miR-409-3p significantly diminished by ˜20-30% the luciferase activity by targeting the PCSK7 3′-UTR (P=0.0013 and P=0.04, respectively). However, miR-320a-3p and miR-224-5p, used as controls, had no significant effect (P0.05, P>0.99, and P=0.91) on luciferase activity. Accordingly, miR-125a-5p, miR-143-3p, and miR-409-3p were chosen for further analysis of their possible direct interactions with the PCSK7 3′-UTR.

### Example 14: Direct Interactions Between miR-125a-5p, miR-143-3p, and miR-409-3p with the 3′-UTR of PCSK7

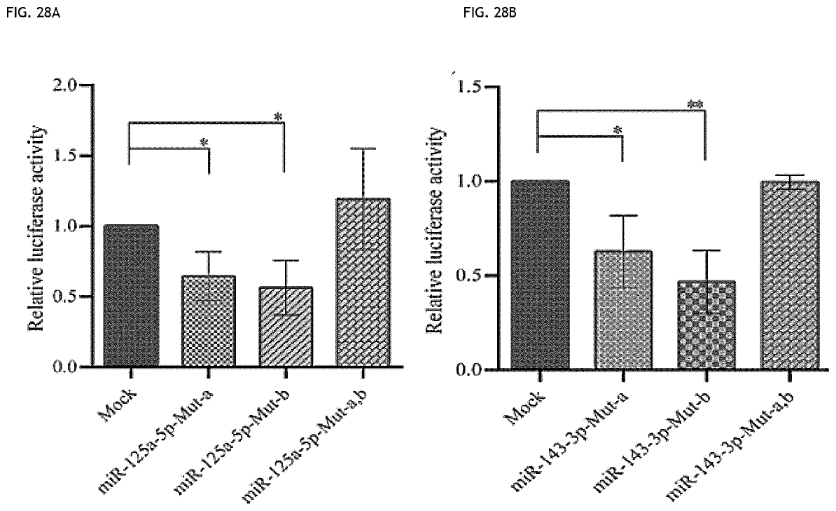

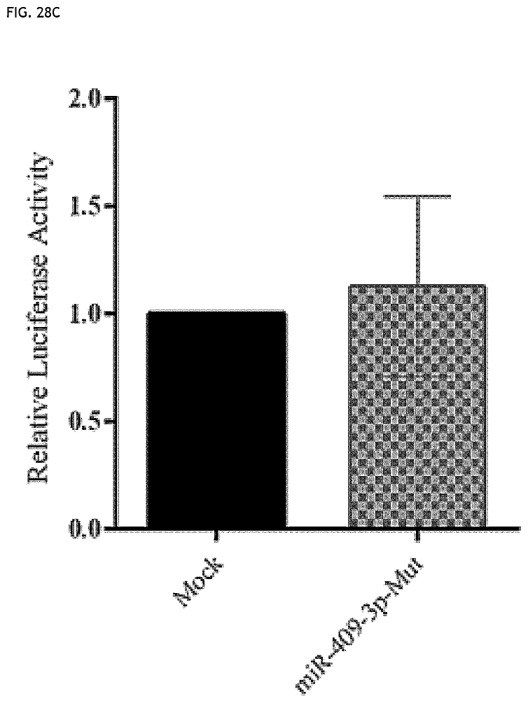

A luciferase reporter assay further confirmed that miR-125a-5p, miR-143-3p, and miR-409-3p directly targeted the 3′-UTR of the PCSK7 transcript since the negative regulatory effect was lost upon the deletion of the miRNA target sites (miR-125a-5p, miR-143-3p, and miR-409-3p) on the 3′-UTR of the PCSK7 reporter plasmid (FIGS. 28A-B). In this regard, three different mutated miR plasmids were constructed for both miR-125a-5p and miR-143-3p. Single-target sites of each miRNA were omitted individually in two separate plasmids, and the two putative binding sites of the miRNAs were deleted in the other plasmid. The data demonstrated that overexpression of miR-125a-5p still decreased the relative luciferase activity in the HEK293T cells co-transfected with the plasmids carrying a mutated form of PCSK7 3′-UTR, containing a single deletion of miR-binding sites (Mut-a and Mut-b), compared with cells co-transfected with the mock counterpart vectors along with the same mutant vectors (Mut-a and Mut-b) (P=0.04 and P=0.01). In contrast, a mutated form of PCSK7 3′-UTR, featuring the deletion of both miRNA target sites (Mut-a,b), failed to significantly decrease the relative luciferase activity in these cells (P=0.24) (FIG. 28A). Like what was observed for miRNA-125a-5p, overexpression of miR-143-3p had no significant effect on luciferase activity when the two putative target sites (Mut-a,b) were omitted from the 3′-UTR of PCSK7 in HEK293T cells (P=0.98). By comparison, luciferase activity was considerably diminished in these cells expressing the miR-143-3p plasmid and mutated vectors carrying only one miRNA response element sequence (Mut-a; P=0.03 and Mut-b; P=0.004) (FIG. 28B). Finally, miR-409-3p overexpression did not lead to a reduction in luciferase activity when the only putative target site of miR-409-3p was absent from the 3′-UTR of PCSK7 (P=0.40) (FIG. 28C).

Overall, the above results confirmed two independent miRNA response elements for both miR-125a-5p and miR-143-3p and one miRNA response element for miR-409-3p as active elements. miR-125a-5p, miR-143-3p, and miR-409-3p could thus regulate the expression of PCSK7 mRNA through direct interactions with these miRNA-binding target sites.

### Example 15: Functional Effects of miR-125a-5p on PCSK7 Activity

Western blot analysis was conducted to determine whether miR-125a-5p affected the functional activity of endogenous PC7 in Huh7 cells. A wild-type human PC7-overexpressing vector was co-transfected with pre-miR-125a-5p and pre-miR-224-5p-overexpressing plasmids in Huh7 cells. Western blot results at 48 hours post-transfection revealed that overexpressed PC7 protein levels fell significantly by ˜80% (P=0.0025) following miR-125a-5p co-expression, compared with the mock-related (pEGFP-C1) and miR-224-5p controls (FIGS. 29A-B). As control, a human PCSK9-overexpressing vector was co-transfected with miR-125a-5p and miR-224-5p vectors in Huh7 cells, whereby these miRNAs had no effect on the expression level of PCSK9 protein (not shown).

Next, Huh7 cells were co-transfected with vectors expressing PC7 and miR-125a-5p at DNA ratios of 1:2 (2×), 1:3 (3×), and 1:5 (5×). The results showed that all three ratios of miR-125a-5p significantly decreased PC7 protein levels by >80%, with no significant PC7-silencing differences between the different amounts of miR-125a-5p (FIGS. 30A-B).

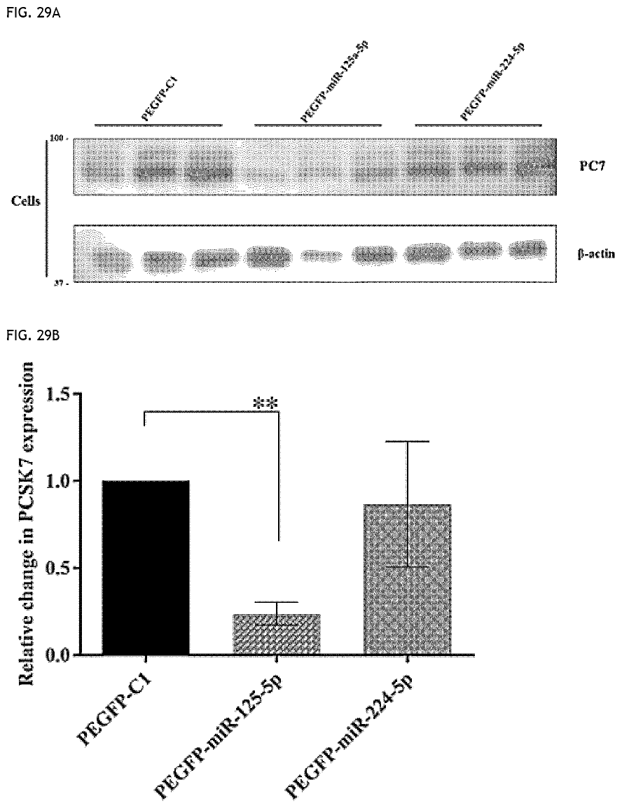

It is known that PC7 specifically cleaves the human type-II membrane-bound hTfR1 into a soluble secreted form thereby enhancing its circulating levels (Guillemot et al., 2013). In addition, a bioinformatics analysis based on Targetscan and UCSC genome browser (miRcode predicted microRNA target sites track) demonstrated that miR-125a-5p does not have any target sites on the 3′-UTR of hTfR1. In agreement, miR-125a-5p that reduced the level of the PC7 protein (FIGS. 29A-B and 30A-B), exerted no effect on the expression level of the hTfR1 protein (FIGS. 31A-B). Accordingly, for the assessment of the functional effect of miR-125a-5p on PC7 activity, hTfR1-V5 was co-expressed with PC7 and different amounts of the miR-125a-5p (2×, 3×, and 5×)-expressing vectors in the Huh7 cells (FIGS. 32A-B). The data show that the presence of miR-125a-5p led to a decline in the ability of PC7 to shed hTfR1 into the media (FIGS. 32A-B). These results demonstrated that miR-125a-5p not only diminished the expression level of PCSK7 mRNA but also functionally abrogated its protease activity on hTfR1.

### Example 16: Silencing Human PC7 with Antisense Oligonucleotides

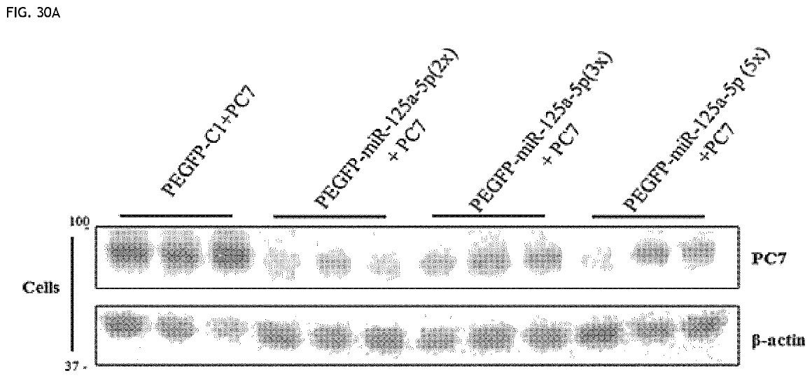

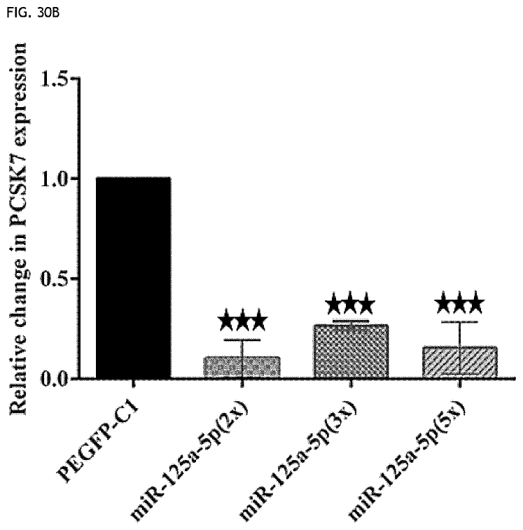

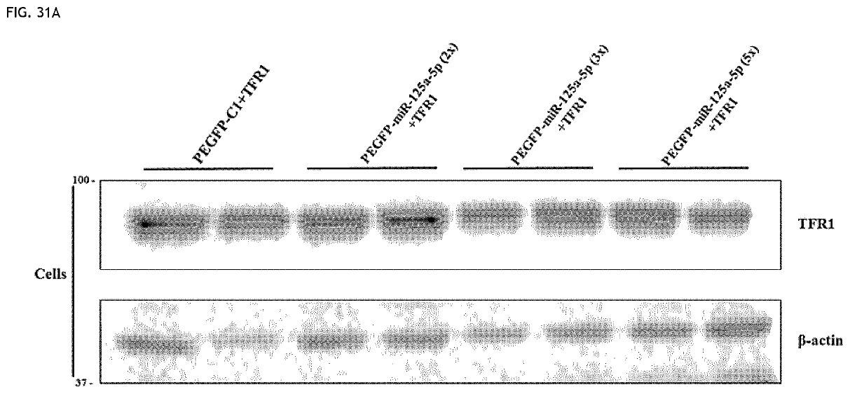

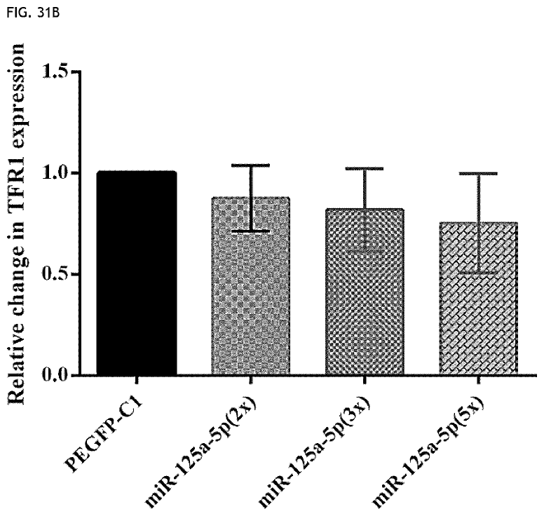

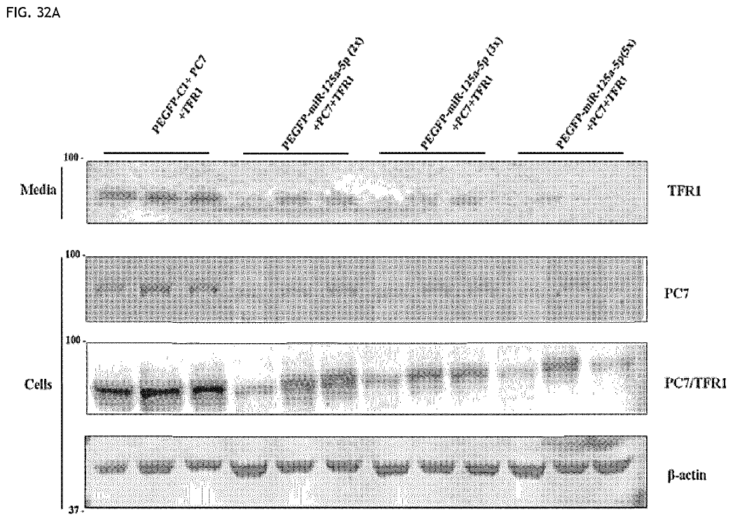

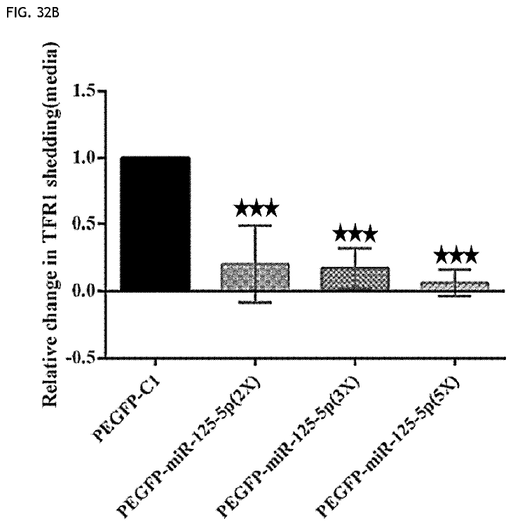

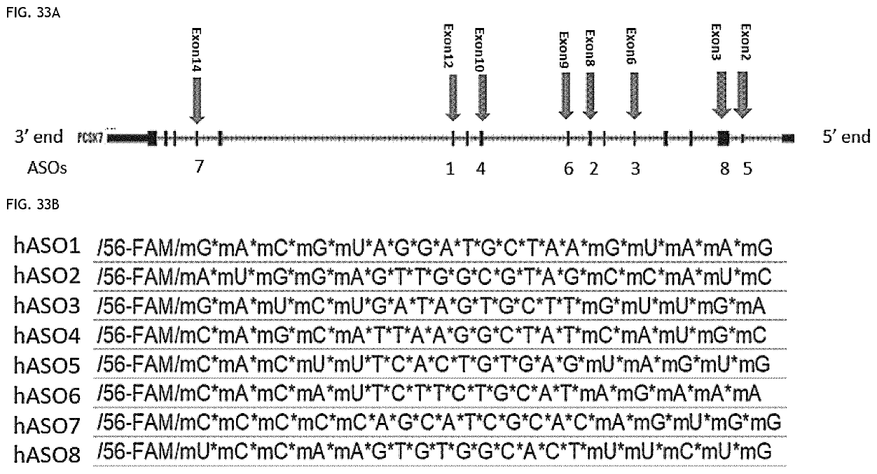

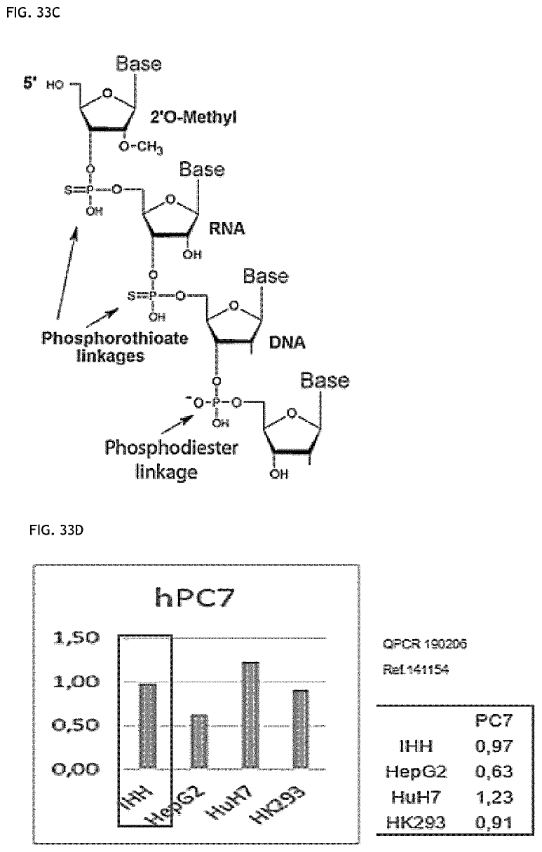

A series of antisense oligos (ASO) were designed against human (h) PC7 genes, each targeting different exons. Two non-targeting ASOs were also designed as controls. From this series, eight were selected and synthesized to test their efficiency in knocking down PC7 (FIGS. 33A-B) (see FIG. 33B for sequences). All oligonucleotides were 20 nucleotides in length and were labeled with a FAM fluorophore on their 5′ end, allowing to track their internalization in cells following transfection. Following the gapmer design, the first 5 nucleotides at the 5′ end and last 5 nucleotides at the 3′ end of each oligonucleotide were modified with 2′O-Methyl moieties, whereas all nucleotides contained phosphorothioate modifications (FIG. 33C).

The mRNA expression levels of PC7 were measured in three different human hepatocyte cell lines (IHH, HepG2, HuH7) and one kidney cell line HEK293. As shown in FIG. 33D, levels of PC7 were highest in HuH7, followed by IHH, HEK293 and HepG2. IHH cells were transfected with 25 nM of each ASO in parallel using Dharmafect™ transfection reagent.

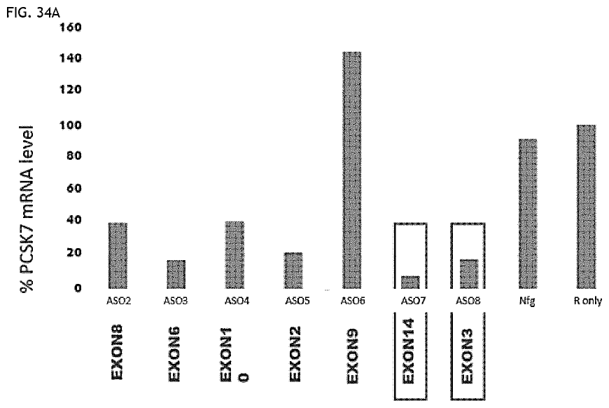

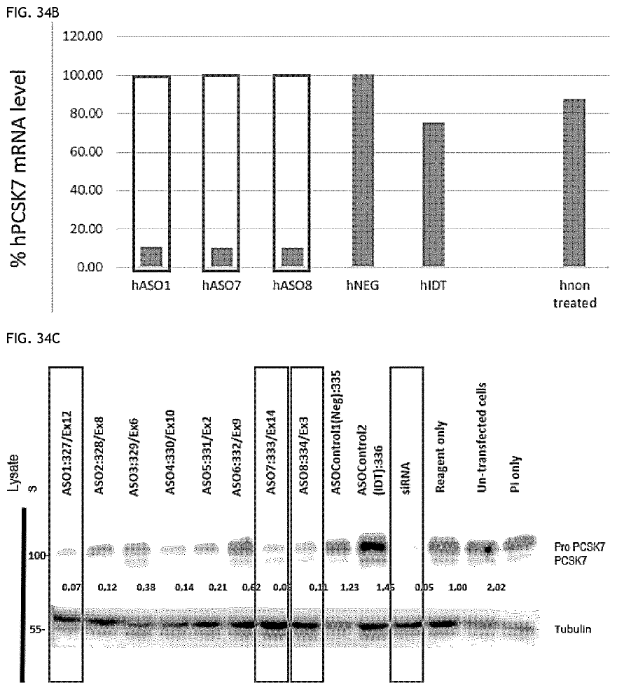

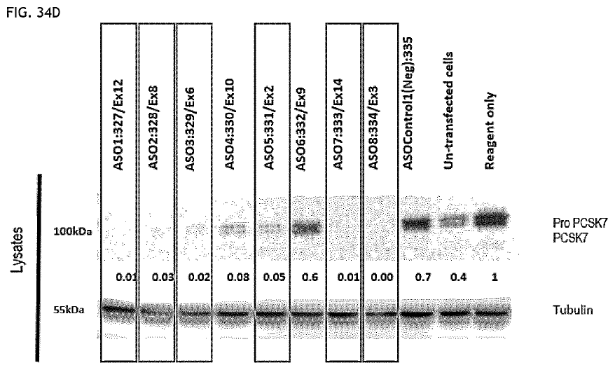

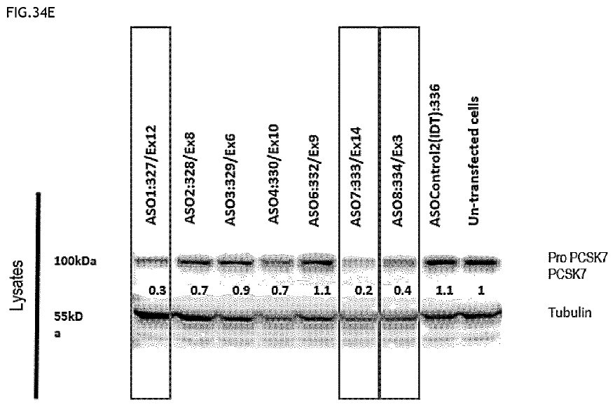

Analysis of FAM fluorescence 24 h after transfection by flow cytometry analysis revealed 84-90% transfection efficiencies. Knockdown efficiencies of each ASO was assessed by RT-qPCR and western blotting of PC7 48 hours after transfection (FIG. 34A). ASOs #1, #7 and #8, targeting exons 12, 14 and 3, respectively, gave the strongest knockdown, achieving >85-90% decrease in PC7 mRNA levels and 89-94% decrease at the protein level, similar to siRNA-mediated knockdown of PC7 (FIG. 34C). Except for ASOs #6 targeting exon 9, all other ASOs tested achieved 62-88% decreased expression of PC7 at the protein level (FIG. 34D). Knockdown of PC7 persisted and was even stronger 72 h after transfection, leading to 92-100% loss of PC7 for 7 out of 8 ASOs tested (FIG. 34D). Even 96 h after transfection, protein levels of PC7 remained at 20-40% for ASOs #1, #7 and #8 compared to non-targeting ASO control levels, whereas levels went back up to 70-90% normal levels with other ASOs (FIG. 34E). This indicates that ASOs #1, #7 and #8 are potent and can decrease PC7 protein levels as efficiently as siRNAs up to 96 h after transfection.

### Example 17: Silencing Mouse Pcsk7 with ASOs

ASOs against different exons of the mouse Pcsk7 gene were also designed (FIGS. 35A-B). Eight ASOs were selected and synthesized following the same chemistry as in Example 16: 20 nt gapmer with 5′FAM labeling) (see FIG. 35B for sequences). 25 nM of each ASO were then transfected in mouse hepatocyte cell line FL83B in parallel, achieving between 37-56% transfection efficiency 24 h post-transfection, as assessed by flow cytometry. Analysis of Pcsk7 RNA levels in bulk cells by RT-qPCR 48 h after transfection revealed 58-60% knockdown efficiency for ASOs #2, #6, #7 and #8 targeting exons 6, 2, 4, and 5, respectively. When performing RT-qPCR only on FAM positive transfected cells, following cell sorting by FACS, actual knockdown efficiencies of ASOs #2, #6, #7 and #8 were between 80-95% (FIG. 36A). Knockdown of Pcsk7 using ASOs #1, #2, and #7 did not affect FL83B cell viability compared to non-targeting ASO (FIG. 36B).

### Example 18: Effect of mASO-2 and mASO-7 and Vitamin E on Reduction of Lipid Droplet Accumulation in Mouse FL38B Hepatocytes

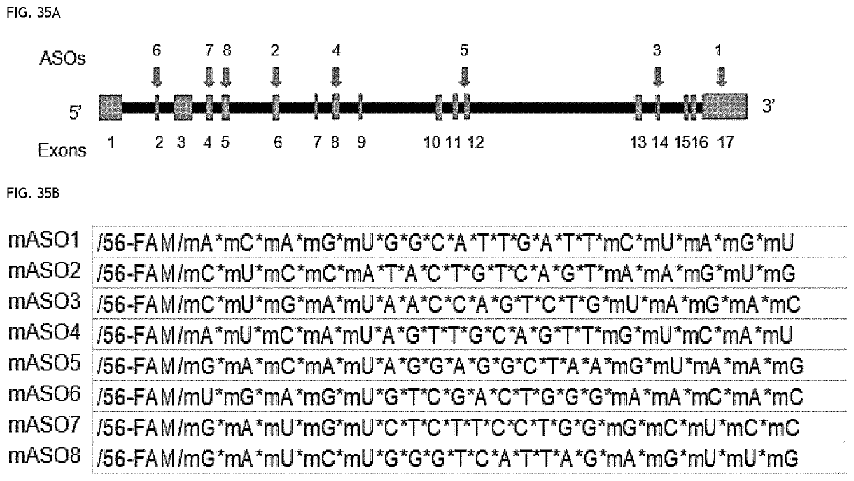

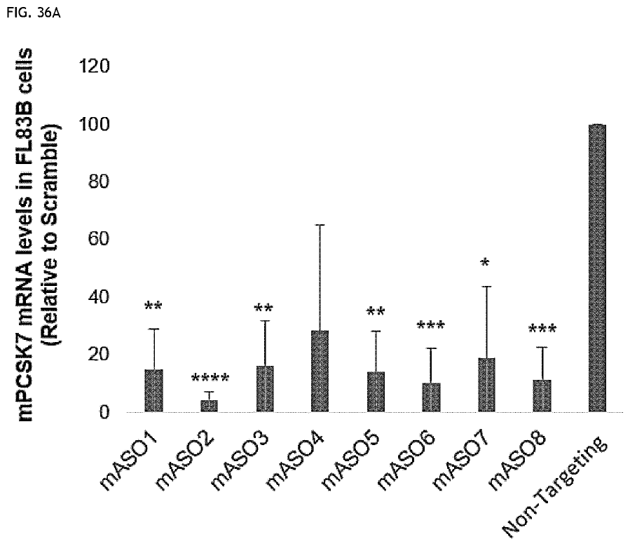

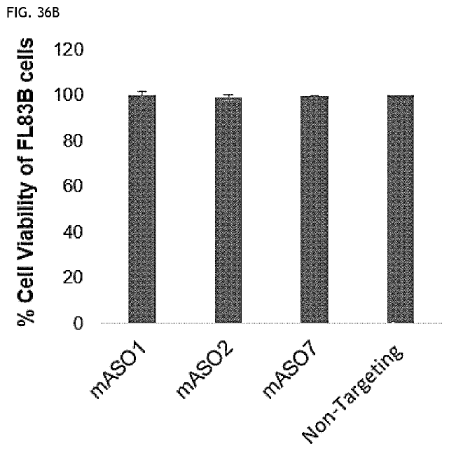

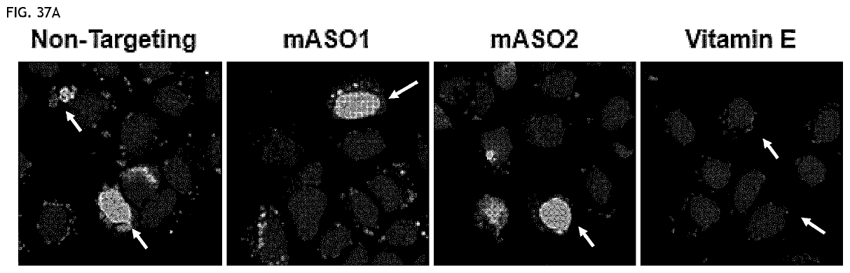

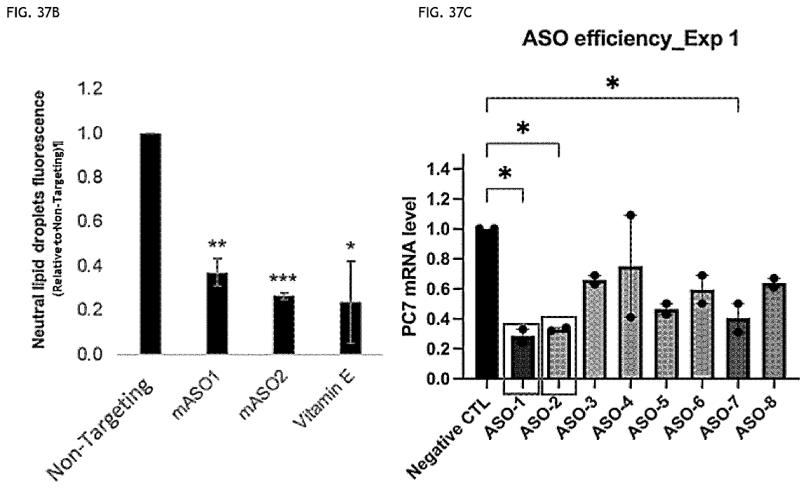

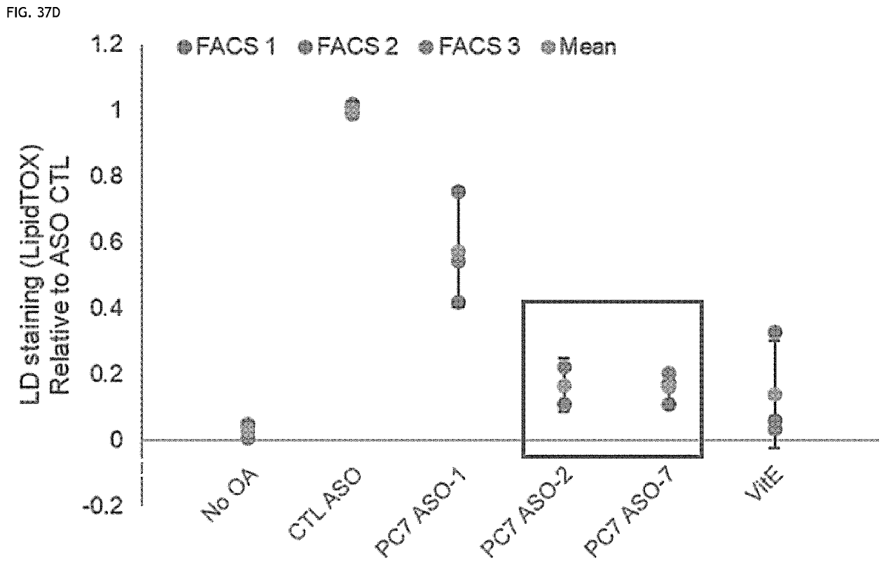

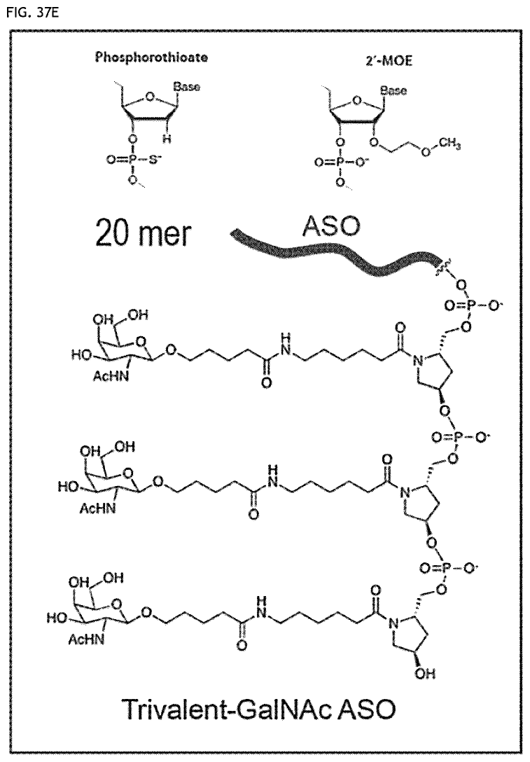

The effect of eight ASOs on Pcsk7 mRNA levels was measured by quantitative PCR (qPCR) (FIG. 37C). Mouse FL38B hepatocyte cells were incubated for 48 h with oleic acid in the presence of either fluorescent (green) mouse mASO-1, mASO-7 or α-tocopherol (vitamin E). The accumulation of lipid droplets (FIGS. 37A, visible as small dots around the nucleus) was significantly reduced in presence of both ASOs as well as vitamin E, as shown in the bar graph quantitative analysis. (FIGS. 37B-37D). Mouse FL38B hepatocyte cells were plated on day 1, ASOs were added on day 2, the media was replaced on day 3 and RNA was extracted on day 4. The same experiment was repeated two independent times, and showed that mASO-1, -2, -7 and vitamin E achieved the best reduction of accumulation of fluorescent triglycerides in cells as estimated by FACS measurement of lipid droplets (LD) by LipidTox™ staining (FIG. 37D). This led the inventors to opt for a modification of ASO-2 and ASO-7 by attachment of trivalent N-acetyl-Galactosamine (FIG. 37E) in order to efficiently target these ASOs to liver hepatocyte, via their binding to the hepatocyte specific receptor ASGR1/2 (see PMID: 34508778 and 34645280).

### Example 19: Treatment Against Diet Induced NAFLD

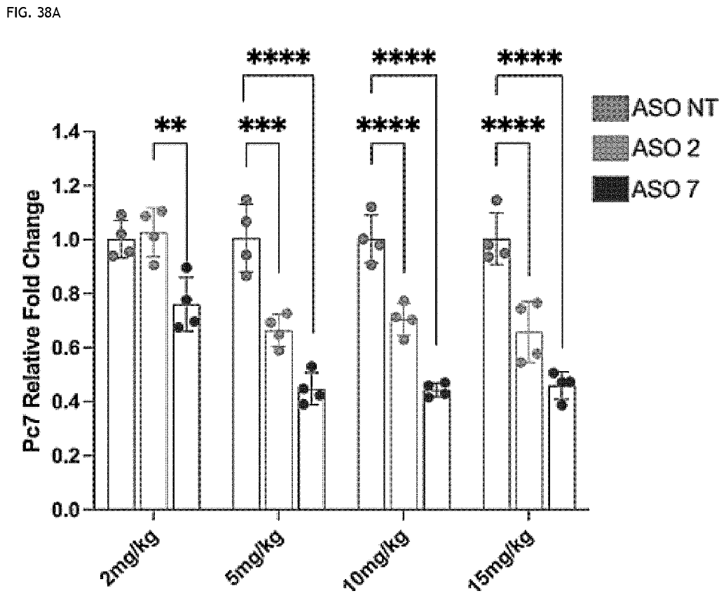

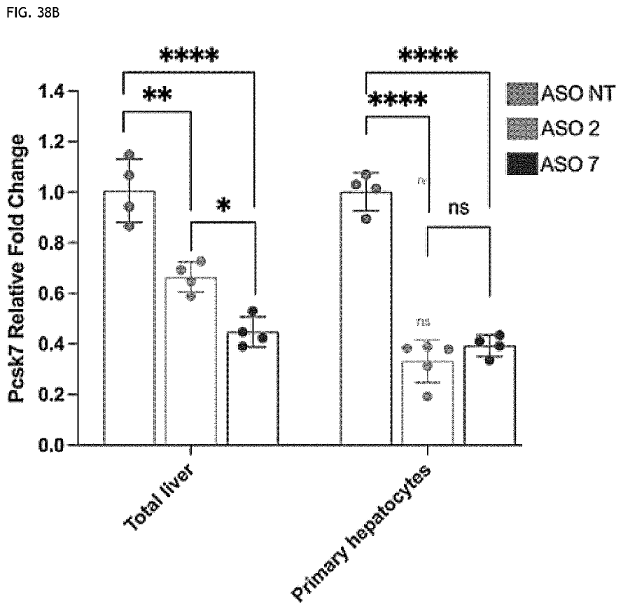

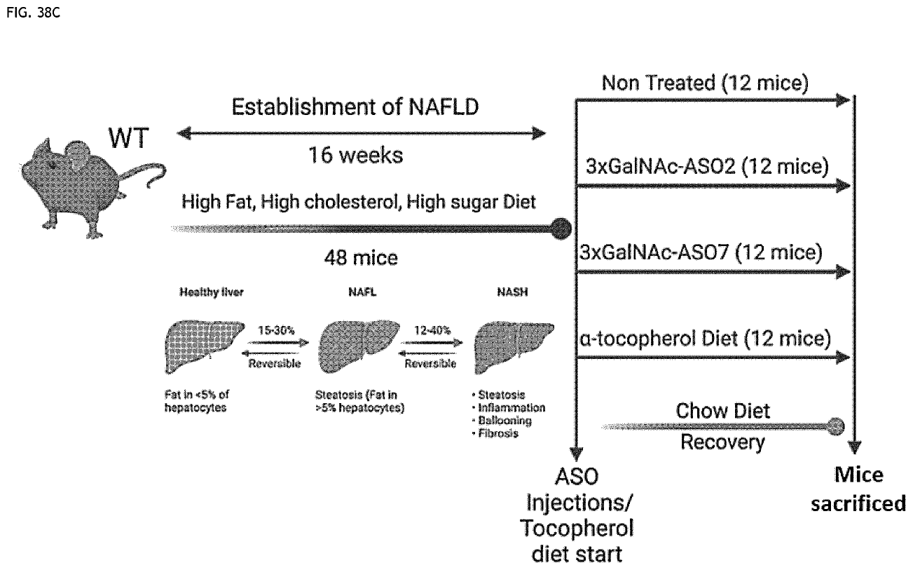

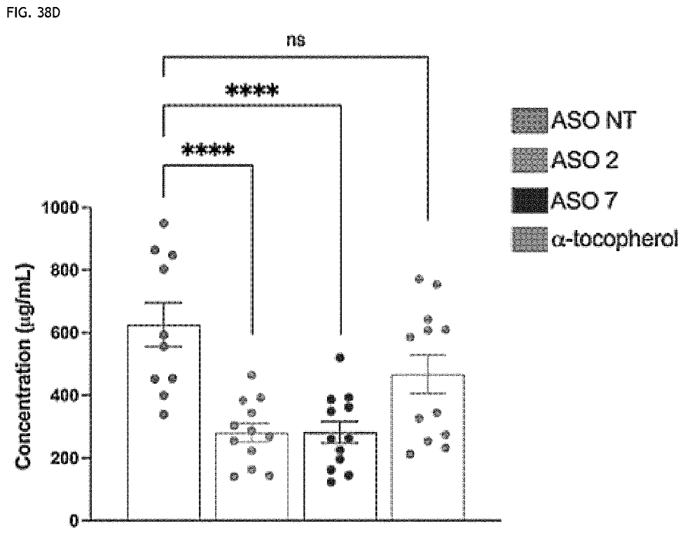

Eight ASOs against mouse Pcsk7 were tested in mouse FL38B hepatocytes and the best ones, namely ASO2 and ASO7, were modified (FIG. 37D) with covalent linkages to trivalent N-acetyl-Galactosamine (GalNac) residues (FIG. 37E). The dose-dependent efficacy of these ASOs following their subcutaneous injection in mice was tested for the decrease in total liver Pcsk7 mRNA at 2, 5, 10 and 15 mg/kg, twice a week for 1 month. At 5 mg/kg ASO-7, and ASO-2, to a lesser extent, reduced the total liver Pcsk7 mRNA by 40-60% (FIG. 38A). Mice were next injected with 5 mg/kg of either GalNac-ASO7 and GalNac-ASO-2, twice a week for 1 month, whereupon the livers and their partially purified primary hepatocytes were analyzed by RT-qPCR for the levels of Pcsk7 mRNA. GalNac-ASO-7 reduced by ˜69% the levels of Pcsk7 mRNA in hepatocytes versus ˜60% in the whole liver, i.e., likely to representing >98% reduction of Pcsk7 mRNA in hepatocytes (FIG. 38B). For GalNac-ASO2 a reduction of ˜69% was observed in the levels of Pcsk7 mRNA in hepatocytes versus ˜34% in the whole liver (FIG. 38B). Thus, both GalNac-ASO7 and GalNac-ASO-2 can reduce by >98% the Pcsk7 mRNA in whole hepatocytes. A program was then set up whereby 8 weeks-old mice were fed a HFFC diet for 16 weeks, after which they were switched to a regular chow diet for another 8 weeks recovery period. The mice were divided into 3 groups of 12 mice each and treated as follows starting from the beginning of the recovery period (FIG. 38C): (1) a control non-treated group; (2) two GalNac-ASO groups subcutaneously injected twice a week for 8 weeks with either GalNac-ASO7 or GalNac-ASO2 at 5 mg/kg; (3) a group fed a chow diet containing Vitamin E (α-tocopherol) at 500 IU/kg of food. This is based on the current guidelines of the American Association for the Study of Liver Diseases for the use of Vitamin E to treat NAFLD patients (PMID: 28714183). The levels of apoB was first analyzed in the plasma of these mice and it was observed that both GalNac-ASO-2 and GalNac-ASO-7 can reduce the circulating levels of apoB by >60% (FIG. 38D), very similar to the plasma levels observed in Pcsk7^{-/-} mice (FIG. 2C), supporting the notion that it is hepatocyte-derived PC7 that primarily regulates circulating apoB levels.

### Example 20: ASO-7 and ASO-2 Reduce Liver Steatosis and NAFLD Much More Effectively than α-Tocopherol

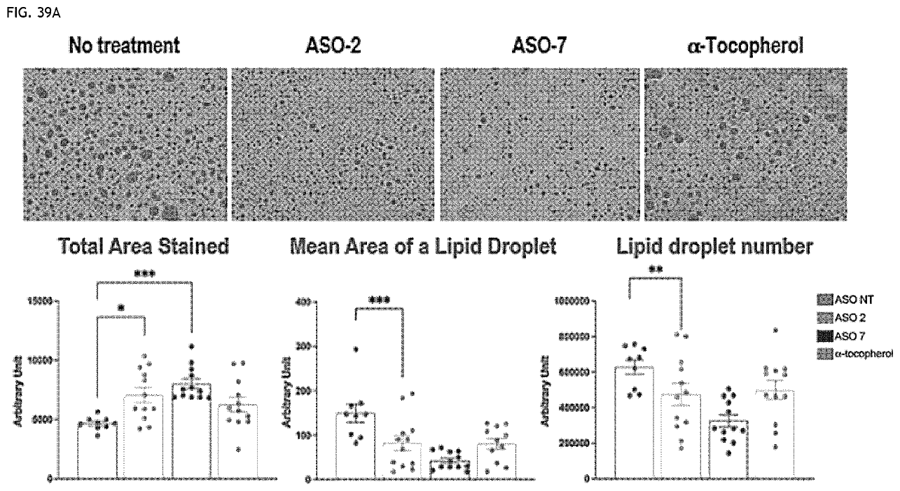

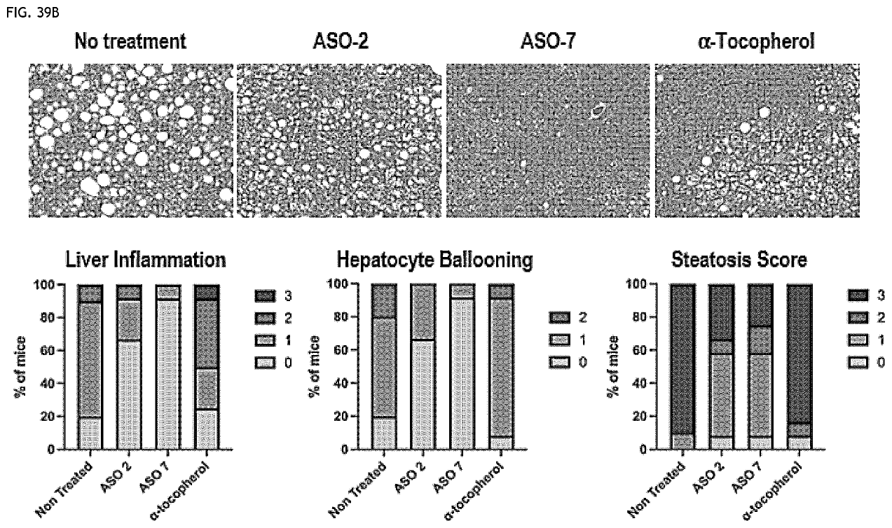

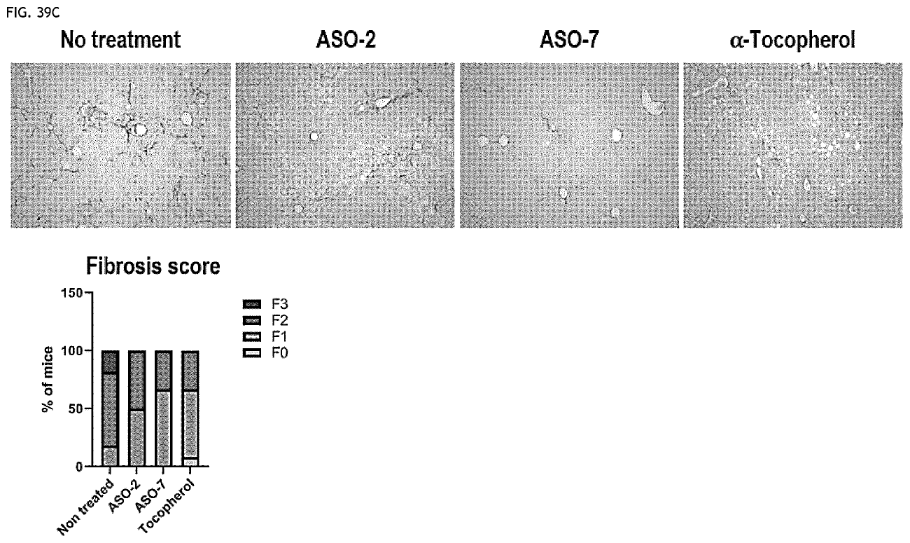

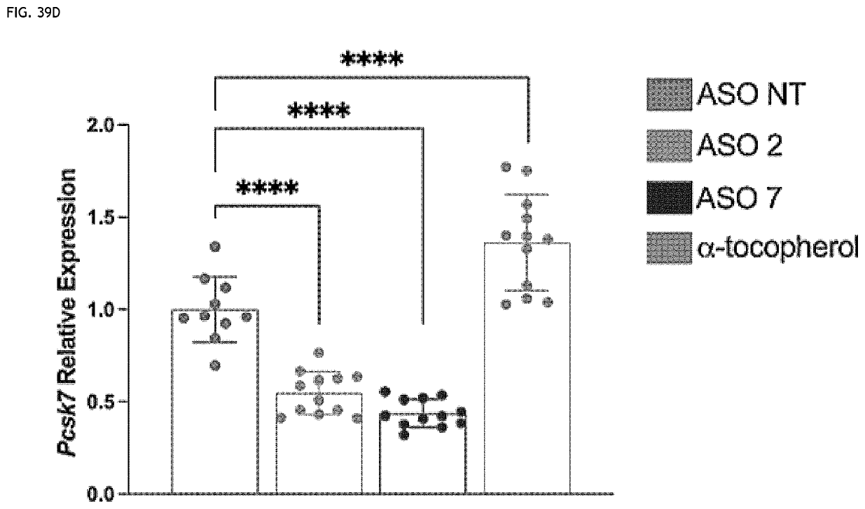

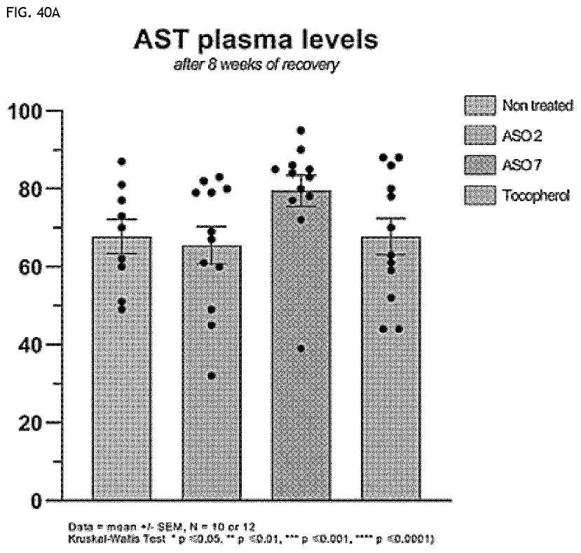

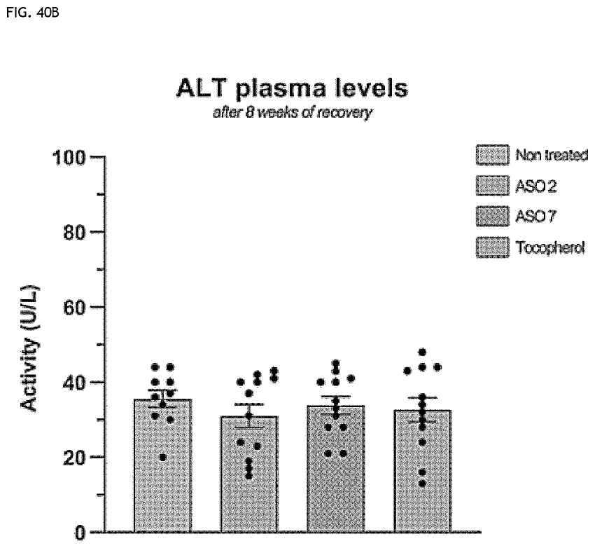

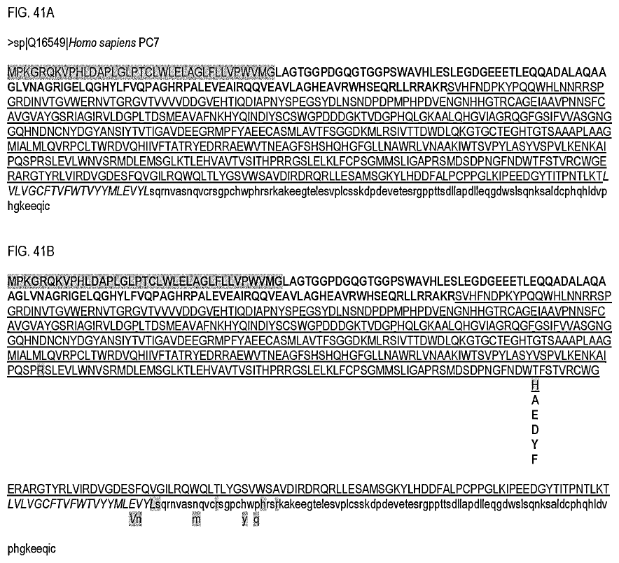

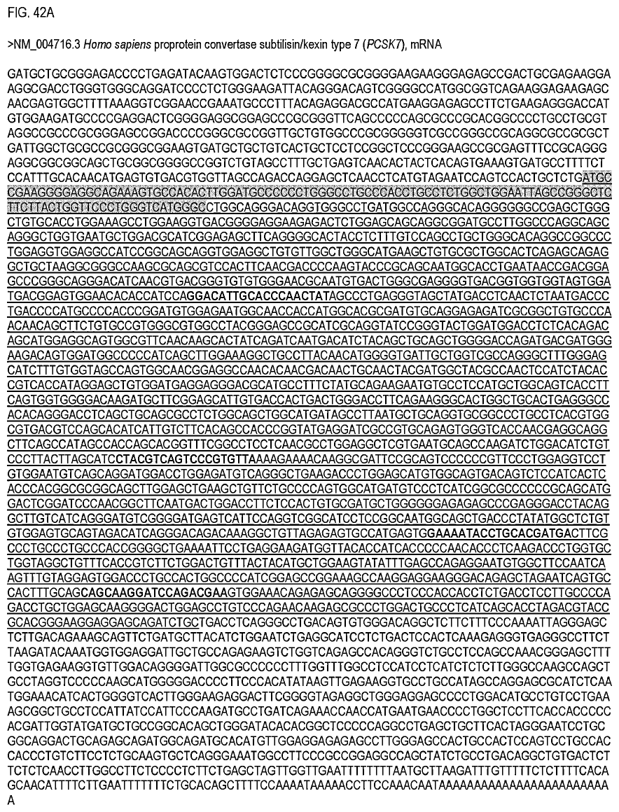

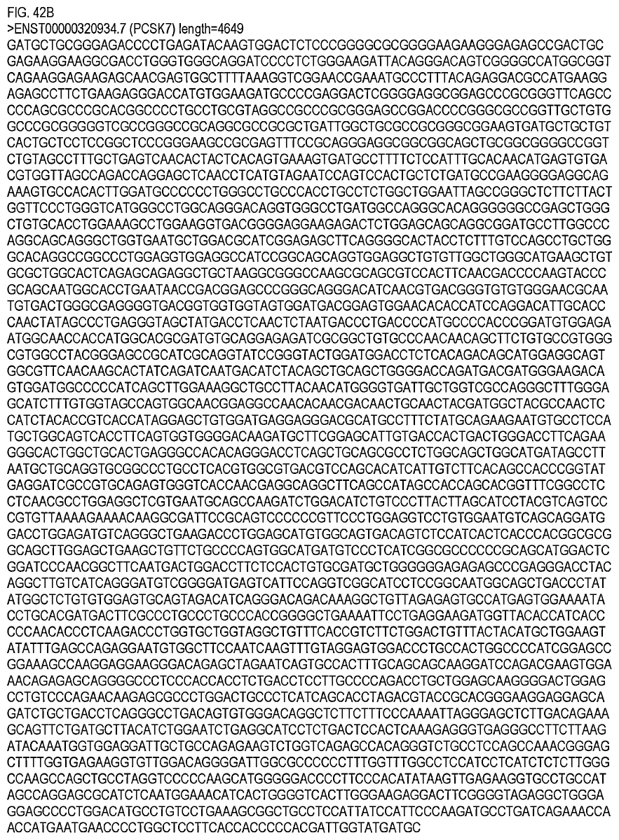

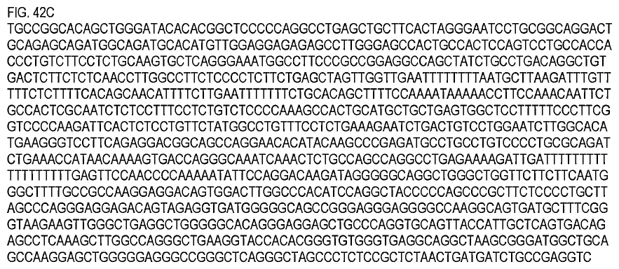

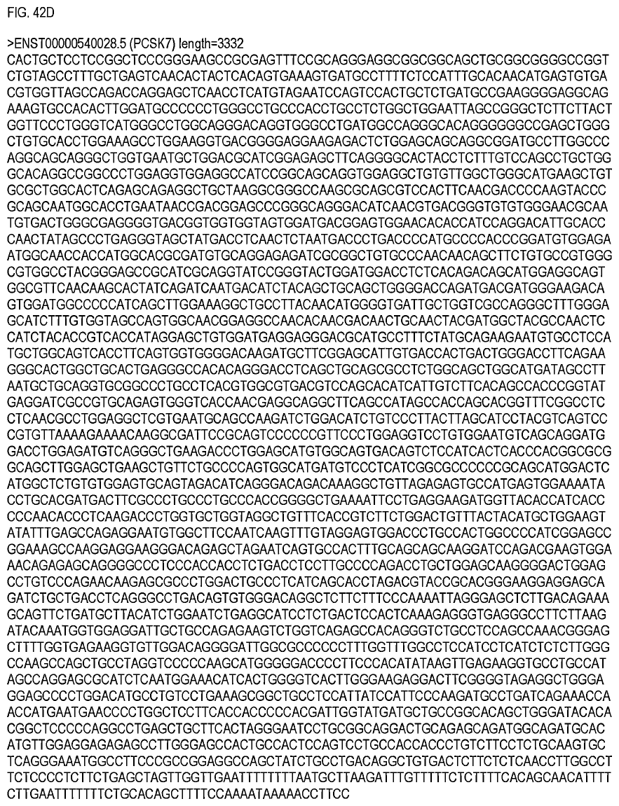

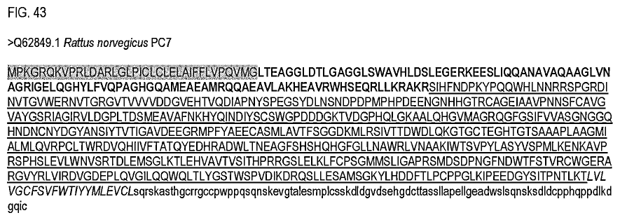

The reduction of LD accumulation in mice treated with GalNac-ASO-7 or GalNac-ASO-2 compared to α-tocopherol was next analyzed (FIG. 39A). In agreement with the data obtained with the whole-body Pcsk7^{-/-} mice (FIGS. 2H, 14H-I), the hepatocyte-specific reduction of Pcsk7 mRNA by GalNac-ASO-7 and GalNac-ASO-2 also significantly reduced the levels of LD following the recovery period, much more effectively than α-tocopherol (FIG. 39A). Thus, GalNac-ASO7 treatment resulted in a mean 47% reduction in the total area of LDs, which are now more abundant (+61%) but smaller (˜67%) (FIG. 39A), likely due to higher lipolysis of TGs induced by the loss of Pcsk7, as discussed before (FIGS. 10A-F, 10B). This was also seen to a lesser extent with GalNac-ASO-2, but significantly less with α-tocopherol (FIG. 39A). These results were corroborated by the analysis of the liver morphology and fat accumulation (FIG. 39B), which were graded blindly by a pathologist. Thus, GalNac-ASO-7 significantly reduced liver inflammation, hepatocyte ballooning and steatosis grade (FIG. 39B) almost to the level of a normal liver (denoted by stage zero). Finally, the extent of fibrosis that typically develop in NAFLD/NASH was also analyzed by Sirius red staining which showed that GalNac-ASO-7 significantly reduced liver fibrosis to a similar extent to that of the antioxidant α-tocopherol (FIG. 39C).

### Example 21: ASO-7 and ASO-2 Reduced Liver Pcsk7 mRNA Levels, Whereas α-Tocopherol Enhanced them

The inventors next compared the levels of Pcsk7 mRNAs in the livers of mice after the 8 weeks recovery period that followed the 16-weeks HFFC diet. GalNac-ASO7 and GalNac-ASO2 reduced total liver Pcsk7 mRNA by 56% and 45% respectively, whereas α-tocopherol in the diet caused a 33% increase in Pcsk7 mRNA levels (FIG. 39D). However, none of these treatments resulted in any damage to the liver or toxicity, as assessed by invariant levels of either AST (Aspartate Aminotransferase) (FIG. 40A) or ALT (Alanine Aminotransferase) (FIG. 40B).

Similar experiments are conducted with apobec-1^{-/-} and Ldlr^{-/-} mice (PMID: 8824235 and 9701246), mice model with close lipid profiles to human.

The scope of the claims should not be limited by the embodiments set forth in the examples but should be given the broadest interpretation consistent with the description as a whole.

### REFERENCES

Abifadel M, et al. Mutations in PCSK9 cause autosomal dominant hypercholesterolemia. Nat Genet 34, 154-156 (2003).
Adeli K, Macri J, Mohammadi A, Kito M, Urade R, Cavallo D. Apolipoprotein B is intracellularly associated with an ER-60 protease homologue in HepG2 cells. J Biol Chem 272, 22489-22494 (1997).
Ashraf Y, Duval S, Sachan V, Essalmani R, Susan-Resiga D, Gerhardy S, Kirchhofer D, Tagliabracci V S, Prat A, Kiss R S, Seidah N G. “Proprotein Convertase 7 (PCSK7) Reduces apoA-V Levels”. FEBS J. 287:3565-3578 (2020).
Ben Djoudi Ouadda A, et al. Ser-Phosphorylation of PCSK9 (Proprotein Convertase Subtilisin-Kexin 9) by Fam20C (Family With Sequence Similarity 20, Member C) Kinase Enhances Its Ability to Degrade the LDLR (Low-Density Lipoprotein Receptor). Arterioscler Thromb Vasc Biol 39, 1996-2013 (2019).
Conlon D M, et al. Inhibition of apolipoprotein B synthesis stimulates endoplasmic reticulum autophagy that prevents steatosis. J Clin Invest 126, 3852-3867 (2016).
Durand L, et al. The motif EXEXXXL in the cytosolic tail of the secretory human proprotein convertase PC7 regulates its trafficking and cleavage activity. J Biol Chem 295, 2068-2083 (2020).
Essalmani R, et al. Furin is the primary in vivo convertase of angiopoietin-like 3 and endothelial lipase in hepatocytes. J Biol Chem 288, 26410-26418 (2013).
Guillemot J, Canuel M, Essalmani R, Prat A, Seidah N G. Implication of the Proprotein Convertases in Iron Homeostasis:
Proprotein Convertase 7 Sheds Human Transferrin Receptor 1 and Furin Activates Hepcidin. Hepatology 57, 2514-2524 (2013).
Kramer, M. F. (2011). Stem-loop RT-qPCR for miRNAs. Current protocols in molecular biology 95, 15.10. 11-15.10. 15. doi. 10.1002/0471142727.mb1510s95.
Wetsel W C, et al. Disruption of the expression of the proprotein convertase PC7 reduces BDNF production and affects learning and memory in mice. Proc Natl Acad Sci USA 110, 17362-17367 (2013).
Xiao, et al. The Guideline of the Design and Validation of MiRNA Mimics. J Cell Physiol 212:285-292, 2007.

1. A method of reducing the level of hepatic triglycerides in a subject in need thereof comprising administering a therapeutically effective amount of a proprotein convertase 7 (PC7) inhibitor to the subject.

2. The method of claim 1, wherein the inhibitor is an anti-PC7 antisense oligonucleotide (ASO), an anti-PC7 microRNA (miRNA) or an anti-PC7 small interfering RNA (siRNA).

3. The method of claim 2, wherein the inhibitor is an ASO.

4. The method of claim 3, wherein the ASO targets a human PC7 RNA DNA transcript, preferably an exon.

5. The method of claim 2, wherein the inhibitor is a siRNA.

6. The method of claim 2, wherein the inhibitor is a miRNA.

7. The method of claim 6, wherein the miRNA targets one or more of human PCSK7 3′-UTR, PCSK7 exon 14, or PCSK7 exon 15.

8. The method of claim 1, wherein the subject has a nonalcoholic fatty liver disease (NAFLD) or is a likely candidate for NAFLD.

9. The method of claim 8, wherein the subject has a nonalcoholic steatohepatitis (NASH) or is a likely candidate for NASH.

10. The method of claim 1, wherein the method further reduces at least one of steatosis score, liver inflammation, hepatocyte ballooning, and liver fibrosis score.

11. The method of claim 1, wherein the subject is a human.

12. A kit for reducing the level of hepatic triglycerides in a subject comprising:
(A) a proprotein convertase 7 (PC7) inhibitor; and
(B) (i) another agent for the prevention or the treatment of a nonalcoholic fatty liver disease (NAFLD) or a symptom thereof;
(ii) a pharmaceutically acceptable carrier;
(iii) instructions to use the kit for reducing the level of hepatic triglycerides; or
(iv) a combination of at least two of (i) to (iii).

13. The kit of claim 12, wherein the PC7 inhibitor is (a) an anti-PC7 antisense oligonucleotide (ASO), (b) an anti-PC7 microRNA (miRNA) against PC7; or (c) an anti-PC7 small interfering RNA (siRNA).

14. The kit of claim 13, wherein the ASO or the siRNA is specific to human PC7.

15. A composition comprising (A) a proprotein convertase 7 (PC7) inhibitor; and (B) (i) another agent for the prevention or the treatment of a nonalcoholic fatty liver disease (NAFLD) or a symptom thereof; (ii) a pharmaceutically acceptable carrier; or (iii) a combination of (i) and (ii).

16. The composition of claim 15, wherein the PC7 inhibitor is (a) an anti-PC7 antisense oligonucleotide (ASO) specific to PC7; (b) an anti-PC7 microRNA (miRNA); or (c) an anti-PC7 small interfering RNA (siRNA).

17. The composition of claim 16, wherein the ASO, miRNA or the siRNA is specific to human PC7.

18. The composition of claim 17, wherein the ASO targets a human PC7 RNA transcript, preferably an exon.

19. The composition of claim 17, wherein the inhibitor is an siRNA.

20. The composition of claim 17, wherein the inhibitor is a miRNA.

21. The composition of claim 17, wherein the miRNA targets one or more of human PCSK7 3′-UTR, PCSK7 exon 14, or PCSK7 exon 15.
